(19) United States
(12) Patent Application Publication
(10) Pub. No.: US 2024/0209054 A1
(43) Pub. Date: Jun. 27, 2024

(54) GIP AGONIST COMPOUNDS AND METHODS

(71) Applicant: Zealand Pharma A/S, Søborg (DK)

(72) Inventors: Anne Pernille Tofteng SHELTON, Søborg (DK); Pia NØRREGAARD, Søborg (DK); Maria Alexandrovna DERYABINA, Søborg (DK); Bjarne Due LARSEN, Søborg (DK); Jacob Ulrik FOG, Søborg (DK)

(21) Appl. No.: 18/371,680

(22) Filed: Sep. 22, 2023

Related U.S. Application Data
(63) Continuation of application No. 17/221,287, filed on Apr. 2, 2021, now Pat. No. 11,814,417.
Application No. 17/221,287 is a continuation of application No. 16/281,347, filed on Feb. 21, 2019, now Pat. No. 11,001,619.
Application No. 16/281,347 is a continuation of application No. 15/521,631, filed on Apr. 25, 2017, now Pat. No. 10,253,078.

(30) Foreign Application Priority Data
Oct. 29, 2014 (DK) 2014 00629
Jul. 4, 2015 (DK) 2015 00381

(51) Int. Cl. C07K 14/575; A61K 38/00; A61K 38/22; A61K 45/06

(52) U.S. Cl. CPC C07K 14/575; A61K 38/22; A61K 45/06; A61K 38/00

(57) ABSTRACT

The present invention relates to acylated GIP analogues which have GIP agonist activity, and their use in the treatment of metabolic disorders.

### SEQUENCE LISTING

The instant application contains a Sequence Listing which has been submitted electronically in XML file format and is hereby incorporated by reference in its entirety. Said XML copy, created on Sep. 27, 2023, is named 50412-100004_SL.xml and is 334,359 bytes in size.

### FIELD OF THE INVENTION

The invention relates to compounds having agonist activity at the GIP receptor, and to their use in the treatment of metabolic disorders.

### BACKGROUND OF THE INVENTION

Diabetes and obesity are increasing health problems globally and are associated with various other diseases, particularly cardiovascular diseases (CVD), obstructive sleep apnea, stroke, peripheral artery disease, microvascular complications and osteoarthritis. There are 246 million people worldwide with diabetes, and by 2025 it is estimated that 380 million will have diabetes. Many have additional cardiovascular risk factors including high/aberrant LDL and triglycerides and low HDL. Cardiovascular diseases account for about 50% of the mortality in people with diabetes, and the morbidity and mortality rates relating to obesity and diabetes underscore the medical need for efficacious treatment options.

Glucose-dependent insulinotropic polypeptide (“GIP”, also known as “gastric inhibitory polypeptide”) is a 42-residue peptide secreted by enteroendocrine K-cells of the small intestine into the bloodstream in response to oral nutrient ingestion. GIP inhibits the secretion of gastric acid, and it has been shown to be a potent stimulant for the secretion of insulin from pancreatic beta cells after oral glucose ingestion (the “incretin effect”) (Creutzfeldt, W., et al, 1979, Diabetologia, 16:75-85).

Insulin release induced by the ingestion of glucose and other nutrients is due to both hormonal and neural factors (Creutzfeldt, W., et al, 1985, Diabetologia, 28:565-573). Several gastrointestinal regulatory peptides have been proposed as incretins, and among these candidates, only GIP and glucagon-like peptide 1 (“GLP-1”) appear to fulfill the requirements to be considered physiological stimulants of postprandial insulin release (Nauck, et al, 1989, J. Clin. Endocrinol Metab., 69:654-662). It has been shown that the combined effects of GIP and GLP-1 are sufficient to explain the full incretin effect of the enteroinsular axis (Fehmann, H. C, et al, 1989, FEBS Lett, 252: 109-112).

As is well known to those skilled in the art, the known and potential uses of GIP are varied and multitudinous. Thus, the administration of the compounds of this invention for purposes of eliciting an agonist effect can have the same effects and uses as GIP itself. These varied uses of GIP may be summarized as follows: treating a disease selected from the group consisting of type 1 diabetes, type 2 diabetes (Visboll, T., 2004, Dan. Med. Bull, 51 :364-70), insulin resistance (WO 2005/082928), obesity (Green, B. D., et al, 2004, Current Pharmaceutical Design, 10:3651-3662), metabolic disorder (Gault, V. A., et al, 2003, Biochem. Biophys. Res. Commun., 308:207-213), central nervous system disease, neurodegenerative disease, congestive heart failure, hypoglycemia, and disorders wherein the reduction of food intake and weight loss are desired. In pancreatic islets, GIP not only enhances insulin secretion acutely, but it also stimulates insulin production through enhancement of proinsulin transcription and translation (Wang, et al, 1996, Mol Cell. Endocrinol, 116:81-87) and enhances the growth and survival of pancreatic beta cells (Trumper, et al, 2003, Diabetes, 52:741-750). In addition to effects on the pancreas to enhance insulin secretion, GIP also has effects on insulin target tissues directly to lower plasma glucose: enhancement of glucose uptake in adipose (Eckel, et al, 1979, Diabetes, 28: 1141-1142) and muscle (O'Harte, et al, 1998, J. Endocrinol, 156:237-243), and inhibition of hepatic glucose production (Elahi, D., et al, 1986, Can. J. Physiol. Pharmacol, 65:A18).

Recently, it has been reported that body weight loss associated with GLP-1 agonist treatment, is enhanced when GLP-1 and GIP are co-administered (Finan, Sci Transl Med. 2013; 5(209):209ra151. Irwin N et al, 2009, Regul Pept; 153: 70-76. Gault et al, 2011, Clin Sci (Lond); 121:107-117). For instance, Finan and colleges demonstrated significant body weight loss in diet-induced obese (DIO) mice after sub-chronic co-administration with an acylated GIP agonist and an acylated GLP-1 agonist. The co-administration decreased body weight and fat mass to a greater extent than either mono-agonist alone. Evidence also suggests that GLP-1 and GIP have additive effects on glycemic control (Gault et al, 2011, Clin Sci (Lond); 121:107-117). A study by Gault et al showed that sub-chronic co-administration with a GLP-1 analogue, and an acylated GIP analogue resulted in greater glucose-lowering and insulinotropic actions during an intraperitoneal glucose tolerance test in ob/ob mice than injection with the GLP-1 agonist or the GIP agonist alone. Thus, GIP agonists may be particular effective in improving glycaemic control and reducing body weight when they are administered in combination with a GLP-1 receptor agonist (as part of the same pharmaceutical formulation or as separate formulations).

The use of unmodified GIP as a therapeutic, however, is limited by the short in vivo half-life of about 2 minutes (Said and Mutt, 1970, Science, 169:1217-1218). In serum, both incretins, GIP and GLP-1, are degraded by dipeptidyl peptidase IV (“DPPIV”). Improving the stability of GIP to proteolysis not only maintains the activity of GIP at its receptor but, more importantly, prevents the production of GIP fragments, some of which act as GIP receptor antagonists (Gault, et al., 2002, J. Endocrinol, 175:525-533). Reported modifications have included protection of the N-terminus of GIP from proteolysis by DPPIV through modification of the N-terminal tyrosine (O'Harte, et al, 2002, Diabetologia, 45: 1281-1291), mutation of the alanine at position 2 (Hinke, et al, 2002, Diabetes, 51:656-661), mutation of glutamic acid at position 3 (Gault, et al, 2003, Biochem. Biophys. Res. Commun., 308:207-213), and mutation of alanine at position 13 (Gault, et al, 2003, Cell Biol. International, 27:41-46),

The following patent applications have been filed related to the effects of GIP analogues on the function of various target organs and their potential use as therapeutic agents:

PCT publication WO 00/58360 discloses peptidyl analogues of GIP which stimulate the release of insulin. In particular, this application discloses specific peptidyl analogues comprising at least 15 amino acid residues from the N-terminal end of GIP(I-42.

PCT publication WO 03/082898 discloses C-terminal truncated fragments and N-terminal modified analogues of GIP, as well as various GIP analogues with a reduced peptide bond or alterations of the amino acids close to the DPPFV-specific cleavage site. This application further discloses analogues with different linkers between potential receptor binding sites of GIP. The compounds of this application are alleged to be useful in treating GIP-receptor mediated conditions, such as non-insulin dependent diabetes mellitus and obesity. Moreover, among other therapeutic effects of the compounds of the present invention as illustrated herein, tighter control of plasma glucose levels may prevent long-term diabetic complications, thereby providing an improved quality of life for patients. In addition to improving blood glucose control, GIP may also enhance GLP-1-mediated body weight loss.

Conjugation of GIP analogues to e.g, PEG(poly ethylene glycol) has been shown to extent in vivo half-life, but potential side-effects of pegylated pharmaceutical products such as inteferon-beta and ribavirin has been reported (J Clin Gastroenterol. 2004 Sep;38(8):717-22, Gut 2006;55:1350-1359 doi:10.1136/gut.2005.076646).

Thus, there still exists a need for improved and safe analogues of GIP, which are stable in formulation and have long in vivo half-life, resulting from decreased susceptibility to proteolysis and decreased clearance, while maintaining binding affinity to a GIP receptor to elicit agonistic effects.

### SUMMARY OF THE INVENTION

The present invention concerns GIP analogues which may have the property of an altered GIP activity, as assessed in in vitro efficacy assays and an altered, preferably increased terminal elimination half-life (T½), as assessed in in vivo studies in mice.

It has been found that GIP receptor agonists of the present invention are superior to existing GIP analogues because the GIP agonists offer long terminal half-lifes. The GIP analogues may thus be used as therapeutics for metabolic disorders including, but not limited to, type 2 diabetes mellitus, obesity and related disorders.

The invention provides in a first aspect a GIP analogue represented by the general Formula I:

(SEQ ID NOs: 42 & 86)
R¹-Tyr-X2-Glu-Gly-Thr-Phe-Ile-Ser-Asp-X10-
X11-X12-Glu-Leu-X15-X16-X17-X18-X19-X20-X21-
Phe-X23-X24-X25-Leu-X27-X28-X29-Y1-Y2-R²
(I)

wherein
R¹ is H—, Ac or pGlu pyroglutamic acid (pGlu; (S)-(−)-2-pyrrolidone-5-carboxylic acid), C₁₋₄ alkyl, acetyl, formyl, benzoyl and trifluoroacetyl,
X2 is Aib, Ala, D-Ala, Gly, Ser, N-Me-Ser, Ac3c, Ac4c or Ac5c;
X10 is Tyr, Leu or Ser;
X11 is Ser or Leu;
X12 is Lys, Ψ or Ile;
X15 is Asp or Glu;
X16 is Ser, Glu, Lys or Ψ;
X17 is Ile, Lys, Gln, Arg or Ψ;
X18 is His, Arg or Ala;
X19 is Gln, Lys, Ala or Glu;
X20 is Gln, Lys, Ala, His or Arg;
X21 is Ala, Leu, Asp or Glu;
X23 is Val or Ile;
X24 is Asn or Glu;
X25 is Tyr or Trp;
X27 is Leu, Glu, Ser, Lys or Val;
X28 is Ala, Ser or Arg;
X29 is Aib, Gly, Ala, Gln, Thr, Ser or Lys or is absent;
Y1 is Lys-Gly, Gly-Pro-Ser-Ser-Gly-Ala-Pro-Pro-Pro-Ser (SEQ ID NO: 43), Gly-Pro-Ser-Ser-Gly-Ala-Pro-Pro-Ser (SEQ ID NO: 44), Pro-Ser-Ser-Gly-Ala-Pro-Pro-Pro-Ser (SEQ ID NO: 45), Pro-Ser-Ser-Gly-Ala-Pro-Pro-Ser (SEQ ID NO: 46), Gly-Lys-Lys-Asn-Asp-Trp-Lys-His-Asn-Ile-Thr-Gin (SEQ ID NO: 47) or absent;
Y2 is Ψ or is absent;
R2 is —NH₂ or —OH;
wherein Ψ is a residue independently selected from Lys, Arg, Orn and Cys and wherein the side chain of said residue is conjugated to a lipophilic substituent;
and wherein the GIP analogue contains one and only one residue Ψ;
or a pharmaceutically acceptable salt or solvate thereof.

In one aspect, R¹ is H—, Ac or pGlu.

Combinations of residues which may be present at some of the variable positions of Formula I include:
Aib2, Asp15, Lys20;
Aib2, Asp15, Arg20;
Aib2, Asp15, Arg20, Ile23;
Aib2, Ile12, Asp15, Arg20, Ile23, Glu24;
Ile12, Asp15, Ile23;
Ile12, Asp15, Ile23, Glu24;
Ile12, Asp15, Ala21, Ile23;
Aib2, Ala21, Ile23, Glu24;
Aib2, Asp15, Ile23;
Aib2, Asp15, Arg20, Ile23, Gln29;
Aib2, Asp15, Arg20, Gly29;
Aib2, Asp15, Ile17, Arg20, Gly29;
Aib2, Asp15, Ile17, Lys20, Gly29;
DAla2, Asp15, Ile23;
DAla2, Asp15, Ile23, Ala28;
Aib2, Asp15, Ile17, Lys20, Ala28;
Asp15, Ile23, Glu24;
N-Me-Ser2, Asp15, Lys20;
N-Me-Ser2, Asp15, Arg20;
N-Me-Ser2, Asp15, Arg20, Ile23;
N-Me-Ser2, Ile12, Asp15, Arg20, Ile23, Glu24;
N-Me-Ser2, Ala21, Ile23, Glu24;
N-Me-Ser2, Asp15, Ile23;
N-Me-Ser2, Asp15, Arg20, Ile23, Gln29;
N-Me-Ser2, Asp15, Arg20, Gly29;
N-Me-Ser2, Asp15, Ile17, Arg20, Gly29;
N-Me-Ser2, Asp15, Ile17, Lys20, Gly29;
N-Me-Ser2, Asp15, Ile23;
N-Me-Ser2, Asp15, Ile23, Ala28;
Ac3c2, Asp15, Lys20;
Ac3c2, Asp15, Arg20;
Ac3c2, Asp15, Arg20, Ile23;
Ac3c2, Ile12, Asp15, Arg20, Ile23, Glu24;
Ac3c2, Ala21, Ile23, Glu24;
Ac3c2, Asp15, Ile23;
Ac3c2, Asp15, Arg20, Ile23, Gln29;
Ac3c2, Asp15, Arg20, Gly29;
Ac3c2, Asp15, Ile17, Arg20, Gly29;
Ac3c2, Asp15, Ile17, Lys20, Gly29;
Ac3c2, Asp15, Ile23;
Ac3c2, Asp15, Ile23, Ala28;
Ac4c2, Asp15, Lys20;
Ac4c2, Asp15, Arg20;
Ac4c2, Asp15, Arg20, Ile23;
Ac4c2, Ile12, Asp15, Arg20, Ile23, Glu24;
Ac4c2, Ala21, Ile23, Glu24;
Ac4c2, Asp15, Ile23;
Ac4c2, Asp15, Arg20, Ile23, Gln29;
Ac4c2, Asp15, Arg20, Gly29;
Ac4c2, Asp15, Ile17, Arg20, Gly29;
Ac4c2, Asp15, Ile17, Lys20, Gly29;
Ac4c2, Asp15, Ile23;
Ac4c2, Asp15, Ile23, Ala28;
Ac5c2, Asp15, Lys20;
Ac5c2, Asp15, Arg20;
Ac5c2, Asp15, Arg20, Ile23;
Ac5c2, Ile12, Asp15, Arg20, Ile23, Glu24;
Ac5c2, Ala21, Ile23, Glu24;
Ac5c2, Asp15, Ile23;
Ac5c2, Asp15, Arg20, Ile23, Gln29;
Ac5c2, Asp15, Arg20, Gly29;
Ac5c2, Asp15, Ile17, Arg20, Gly29;
Ac5c2, Asp15, Ile17, Lys20, Gly29;
Ac5c2, Asp15, Ile23; or
Ac5c2, Asp15, Ile23, Ala28.

The invention provides in a further aspect a GIP analogue represented by the general Formula II:

(SEQ ID NOs: 48 & 87)
R¹-Tyr-X2-Glu-Gly-Thr-Phe-Ile-Ser-Asp-Tyr-
Ser-X12-Glu-Leu-X15-X16-X17-X18-X19-X20-X21-
Phe-X23-X24-X25-Leu-X27-X28-X29-Y1-Y2-R²
(II)

wherein
R¹ is H—, Ac or pGlu;
X2 is Aib, Ala, D-Ala or Gly;
X12 is Lys, Y or Ile;
X15 is Asp or Glu;
X16 is Ser, Glu, Lys or 4;
X17 is Ile, Lys, Gln, Arg or 4;
X18 is His, Arg or Ala;
X19 is Gln or Ala;
X20 is Gln, Lys, Ala, His or Arg;
X21 is Ala, Asp or Glu;
X23 is Ile or Val;
X24 is Asn or Glu;
X25 is Tyr or Trp;
X27 is Leu, Glu, Ser, Lys or Val;
X28 is Ala, Ser or Arg;
X29 is Aib, Gly, Ala, Gln, Thr, Ser or Lys or is absent;
Y1 is Lys-Gly, Gly-Pro-Ser-Ser-Gly-Ala-Pro-Pro-Pro-Ser (SEQ ID NO: 43), Gly-Pro-Ser-Ser-Gly-Ala-Pro-Pro-Ser (SEQ ID NO: 44), Pro-Ser-Ser-Gly-Ala-Pro-Pro-Pro-Ser (SEQ ID NO: 45), Pro-Ser-Ser-Gly-Ala-Pro-Pro-Ser (SEQ ID NO: 46), Gly-Lys-Lys-Asn-Asp-Trp-Lys-His-Asn-Ile-Thr-Gln (SEQ ID NO: 47) or absent;
Y2 is Ψ or is absent;
R² is —NH₂ or —OH;
wherein Ψ is a Lys residue wherein the side chain of said Lys residue is conjugated to a lipophilic substituent;
and wherein the GIP analogue contains one and only one residue Ψ;
or a pharmaceutically acceptable salt or solvate thereof.

Combinations of residues which may be present at some of the variable positions of Formula II include:
Aib2, Lys12, Asp15, Lys20;
Aib2, Lys12, Asp15, Arg20;
Aib2, Asp15, Arg20;
Aib2, Ile 12, Asp15, Arg20, Glu24;
Ile12, Asp15, Ile23;
Ile12, Asp15, Glu24;
Ile12, Asp15, Ala21;
Aib2, Lys12, Ala21, Glu24;
Aib2, Lys12, Asp15;
Aib2, Lys, 12, Asp15, Arg20, Gln29;
Aib2, Lys, 12, Asp15, Arg20, Gly29;
Aib2, Lys12, Asp15, Ile17, Arg20, Gly29;
Aib2, Asp15, Ile17, Lys20, Gly29;
DAla2, Asp15;
DAla2, Asp15, Ala28;
Aib2, Asp15, Ile17, Lys20, Ala28;
Asp15, Glu24;
Ala2, Lys12, Asp15, Lys20;
Ala2, Lys12, Asp15, Arg20;
Ala2, Asp15, Arg20;
Ala2, Ile 12, Asp15, Arg20, Glu24;
Ala2, Ile12, Asp15, Ile23;
Ala2, Ile12, Asp15, Glu24;
Ala2, Ile12, Asp15, Ala21;
Ala2, Lys12, Ala21, Glu24;
Ala2, lys12, Asp15;
Ala2, Lys12, Asp15, Arg20, Gln29;
Ala2, Lys12, Asp15, Arg20, Gly29;
Ala2, Lys12, Asp15, Ile17, Arg20, Gly29;
Ala2, Asp15, Ile17, Lys20, Gly29;
Ala2, Asp15;
Ala2, Asp15, Ala28;
Ala2, Asp15, Ile17, Lys20, Ala28;
Gly2, Lys12, Asp15, Lys20;
Gly2, Lys12, Asp15, Arg20;
Gly2, Asp15, Arg20;
Gly2, Ile12, Asp15, Arg20, Glu24;
Gly2, Ile12, Asp15, Ile23;
Gly2, Ile12, Asp15, Glu24;
Gly2, Ile12, Asp15, Ala21;
Gly2, Lys12, Ala21, Glu24;
Gly2, Lys12, Asp15;
Gly2, Lys12, Asp15, Arg20, Gln29;
Gly2, Lys 12, Asp15, Arg20, Gly29;
Gly2, Lys12, Asp15, Ile17, Arg20, Gly29;
Gly2, Asp15, Ile17, Lys20, Gly29;
Gly2, Asp15;
Gly2, Asp15, Ala28;
Gly2, Asp15, Ile17, Lys20, Ala28; or
Gly2, Asp15, Glu24.

The invention provides in a further aspect a GIP analogue represented by the general Formula III:

(SEQ ID NOs: 50 & 88)
R¹-Tyr-Aib-Glu-Gly-Thr-Phe-Ile-Ser-Asp-Tyr-
Ser-Ile-Glu-Leu-X15-X16-X17-X18-X19-X20-X21-
Phe-Val-X24-X25-Leu-Leu-Ala-X29-Y1-Y2-R²
(III)

wherein
R¹ is H—, Ac or pGlu;
X15 is Asp or Glu;
X16 is Lys or Ψ;
X17 is Ile or Ψ;
X18 is His or Ala;
X19 is Gln or Ala;
X20 is Gln, Lys or Arg;
X21 is Ala, Asp or Glu;
X24 is Asn or Glu;
X25 is Tyr or Trp;
X29 is Gln or is absent;
Y1 is Lys-Gly, Gly-Pro-Ser-Ser-Gly-Ala-Pro-Pro-Pro-Ser (SEQ ID NO: 43), Gly-Pro-Ser-Ser-Gly-Ala-Pro-Pro-Ser (SEQ ID NO: 44), Pro-Ser-Ser-Gly-Ala-Pro-Pro-Pro-Ser (SEQ ID NO: 45), Pro-Ser-Ser-Gly-Ala-Pro-Pro-Ser (SEQ ID NO: 46), Gly-Lys-Lys-Asn-Asp-Trp-Lys-His-Asn-Ile-Thr-Gln (SEQ ID NO: 47) or absent;
Y2 is Ψ or is absent;
R² is —NH₂ or —OH;
wherein Ψ is a residue independently selected from Lys, Arg, Orn and Cys and wherein the side chain of said residue is conjugated to a lipophilic substituent;
and wherein the GIP analogue contains one and only one residue w;
or a pharmaceutically acceptable salt or solvate thereof.

Combinations of residues which may be present at some of the variable positions of Formula III include:
Asp15, Lys20;
Asp15, Arg20;
Asp15, Arg20, Glu24;
Asp15, Lys 16;
Asp15, Lys 16, Glu24;
Asp15, Ψ16, Ala21;
Ala21, Glu24;
Asp15, Arg20, Gln29;
Asp15, Arg20, Gly29;
Asp15, Ile17, Arg20, Gly29;
Asp15, Ile17, Lys20, Gly29;
Asp15Ala28;
Asp15, Ile17, Lys20, Ala28;
Asp15, Ile23, Glu24;
Asp15, Ψ17, Lys20;
Asp15, Ψ17, Arg20;
Asp15, Ψ17, Arg20
Asp15, Ψ17, Arg20, Glu24;
Asp15, Lys16, Ψ17;
Asp15, Lys16, Ψ17, Glu24;
Asp15, Ψ17, Ala21;
Ala21, Ψ17, Glu24;
Asp15, Asp15, Ψ17, Arg20, Gln29;
Asp15, Ψ17, Arg20, Gly29;
Asp15, Ile17, Arg20, Gly29;
Asp15, Ile17, Lys20, Gly29;
Asp15; Ψ17;
Asp15, Ψ17, Ala28;
Asp15, Ile17, Lys20, Ala28; or
Asp15, Ψ17, Ile23, Glu24.

The GIP-analogue may have the formula R1-Z—R2 where R1 and R2 are as defined above and Z has the sequence:

(SEQ ID NO: 51)
Y-Aib-EGTFISDYSIELDKΨHQQDFVNWLLAQGPSSGAPPPS;
(SEQ ID NO: 52)
Y-Aib-EGTFISDYSIELDΨIHQQDFVNWLLAQGPSSGAPPPS;
(SEQ ID NO: 53)
Y-Aib-EGTFISDYSIELEKΨHQQDFVNWLLAQGPSSGAPPPS;
(SEQ ID NO: 54)
Y-Aib-EGTFISDYSIELDKIHQQDFVNWLLAQGPSSGAPPPSΨ;
(SEQ ID NO: 54)
Y-Aib-EGTFISDYSIELDKIHQQDFVNWLLAQGPSSGAPPPSΨ
(SEQ ID NO: 55)
Y-Aib-EGTFISDYSIELDKIHQQDFVNWLLAQΨ;
(SEQ ID NO: 56)
Y-Aib-EGTFISDYSIELDKIHQQDFVNWLLAQKGΨ;
(SEQ ID NO: 57)
Y-Aib-EGTFISDYSIELDKΨHQQDFVNYLLAQGPSSGAPPPS;
(SEQ ID NO: 58)
Y-Aib-EGTFISDYSIELDKΨHQQDFVNWLLAQGPSSGAPPPS;
(SEQ ID NO: 59)
Y-Aib-EGTFISDYSIELDKΨAAQDFVNWLLAQGPSSGAPPPS;
(SEQ ID NO: 60)
Y-Aib-EGTFISDYSIELEKΨAAKEFVNWLLAQGPSSGAPPPS;
(SEQ ID NO: 61)
Y-Aib-EGTFISDYSIELEKΨAQRAFVEWLLAQGPSSGAPPPS;
(SEQ ID NO: 62)
Y-Aib-EGTFISDYSIELEKIAQRAFVEWLLAQGPSSGAPPPSΨ;
(SEQ ID NO: 63)
Y-Aib-EGTFISDYSIELEKIAQRAFVEWLLAQΨ;
(SEQ ID NO: 64)
Y-Aib-EGTFISDYSIELDKΨAAQDFVNWLLAGPSSGAPPPS;
(SEQ ID NO: 65)
Y-Aib-EGTFISDYSIELDKIAAQDFVNWLLAGPSSGAPPPSΨ;
(SEQ ID NO: 66)
Y-Aib-EGTFISDYSIELDKΨPAQRAFVEWLLAQGPSSGAPPPS;
(SEQ ID NO: 67)
Y-Aib-EGTFISDYSIELDKΨPAQRAFIEWLLAQGPSSGAPPPS;
(SEQ ID NO: 68)
Y-Aib-EGTFISDYSIELDKIAQRAFIEWLLAGPSSGAPPPSKΨ;
(SEQ ID NO: 69)
Y-Aib-EGTFISDYSIELDKIAQKEFIEWLLAGPSSGAPPPSKΨ;
(SEQ ID NO: 70)
Y-Aib-EGTFISDYSIELDKIAAQDFIEWLLAGPSSGAPPPSKΨ;
(SEQ ID NO: 71)
Y-Aib-EGTFISDYSIELDKIAAQDFVEWLLAGPSSGAPPPSKΨ;
(SEQ ID NO: 72)
Y-Aib-EGTFISDYSIELDKIAQRAFIEWLLAQGPSSGAPPPSKΨ;
(SEQ ID NO: 73)
Y-Aib-EGTFISDYSIELDKΨAAQAFVNWLLAGPSSGAPPPS;
(SEQ ID NO: 74)
Y-Aib-EGTFISDYSIELDKΨAAQDFVNWLLAAGPSSGAPPPS;
(SEQ ID NO: 75)
Y-Aib-EGTFISDYSIELDKΨAAQDFINWLLAGPSSGAPPPS;
(SEQ ID NO: 76)
Y-Aib-EGTFISDYSIELDKΨAAQDFIEWLLAGPSSGAPPPS;
(SEQ ID NO: 77)
Y-Aib-EGTFISDYSIELDKΨAAQDFIEWLLAGPSSGAPPPS;
(SEQ ID NO: 78)
Y-Aib-EGTFISDYSIELDKΨIAQRAFIEWLLAQGPSSGAPPPS;
(SEQ ID NO: 79)
Y-Aib-EGTFISDYSΨELDKIAQRAFIEWLLAQGPSSGAPPPS;
(SEQ ID NO: 80)
Y-DAlA-EGTFISDYSIELDKΨAQRAFIEWLLAQGPSSGAPPPS;
(SEQ ID NO: 81)
Y-DAlA-EGTFISDYSIELDKIAAQDFIEWLLAGPSSGAPPPSKΨ;
(SEQ ID NO: 82)
Y-Aib-EGTFISDYSIELDKΨAAQDFIEWLLAQGPSSGAPPPS;
(SEQ ID NO: 83)
Y-Aib-EGTFISDYSIELDKΨAAQDFINWLLAQGPSSGAPPPS;
or
(SEQ ID NO: 84)
Y-Aib-EGTFISDYSIELDKΨAAQAFIEWLLAQGPSSGAPPPS.

The GIP-analogue may have the formula R1-Z—R2 where R1 and R2 are as defined above and Z has the sequence

(SEQ ID NO: 1)
Y-Aib-EGTFISDYSIELDK-K(Hexadecanoyl-isoGlu)-
HQQDFVNWLLAQGPSSGAPPPS;
(SEQ ID NO: 2)
Y-Aib-EGTFISDYSIELD-K(Hexadecanoyl-isoGlu)-
IHQQDFVNWLLAQGPSSGAPPPS;
(SEQ ID NO: 3)
Y-Aib-EGTFISDYSIELEK-K(Hexadecanoyl-isoGlu)-
HQQDFVNWLLAQGPSSGAPPPS;
(SEQ ID NO: 4)
Y-Aib-EGTFISDYSIELDKIHQQDFVNWLLAQGPSSGAPPPS-
K([19-carboxy-nonadecanoyl]-isoGlu-Peg3-Peg3);
(SEQ ID NO: 5)
Y-Aib-EGTFISDYSIELDKIHQQDFVNWLLAQGPSSGAPPPS-
K(Hexadecanoyl-isoGlu);
(SEQ ID NO: 6)
Y-Aib-EGTFISDYSIELDKIHQQDFVNWLLAQ-K
(Hexadecanoyl-isoGlu);
(SEQ ID NO: 7)
Y-Aib-EGTFISDYSIELDKIHQQDFVNWLLAQKG-
K(Hexadecanoyl-isoGlu);
(SEQ ID NO: 8)
Y-Aib-EGTFISDYSIELDK-K([19-carboxy-nonadecanoyl]-
isoGlu-Peg3-Peg3)-HQQDFVNYLLAQGPSSGAPPPS;
(SEQ ID NO: 9)
Y-Aib-EGTFISDYSIELDK-K([19-carboxy-nonadecanoyl]-
isoGlu-Peg3-Peg3)-HQQDFVNWLLAQGPSSGAPPPS;
(SEQ ID NO: 10)
Y-Aib-EGTFISDYSIELDK-K([19-carboxy-nonadecanoyl]-
isoGlu-Peg3-Peg3)-AAQDFVNWLLAQGPSSGAPPPS;
(SEQ ID NO: 11)
Y-Aib-EGTFISDYSIELEK-K([19-carboxy-nonadecanoyl]-
isoGlu-Peg3-Peg3)-AAKEFVNWLLAQGPSSGAPPPS;
(SEQ ID NO: 12)
Y-Aib-EGTFISDYSIELEK-K([19-carboxy-nonadecanoyl]-
isoGlu-Peg3-Peg3)-AQRAFVEWLLAQGPSSGAPPPS;
(SEQ ID NO: 13)
Y-Aib-EGTFISDYSIELEKIAQRAFVEWLLAQGPSSGAPPPS-
K([19-carboxy-nonadecanoyl]-isoGlu-Peg3-Peg3);
(SEQ ID NO: 14)
Y-Aib-EGTFISDYSIELEKIAQRAFVEWLLAQ-K([19-carboxy-
nonadecanoyl]-isoGlu-Peg3-Peg3);
(SEQ ID NO: 15)
H-Y-Aib-EGTFISDYSIELDK-K([19-carboxy-nonadecanoyl]-
isoGlu-Peg3-Peg3)-AAQDFVNWLLAGPSSGAPPPS;
(SEQ ID NO: 16)
Y-Aib-EGTFISDYSIELDKIAAQDFVNWLLAGPSSGAPPPS-
K([19-carboxy-nonadecanoyl]-isoGlu-Peg3-Peg3);
(SEQ ID NO: 17)
Y-Aib-EGTFISDYSIELDK-K([19-Carboxy-nonadecanoyl]-
isoGlu-Peg3-Peg3)-AQRAFVEWLLAQGPSSGAPPPS;
(SEQ ID NO: 18)
Y-Aib-EGTFISDYSIELDK-K([19-Carboxy-nonadecanoyl]-
isoGlu-Peg3-Peg3)-AQRAFIEWLLAQGPSSGAPPPS;
(SEQ ID NO: 19)
Y-Aib-EGTFISDYSIELDK-K((19-Carboxy-nonadecanoyl)-
[(Piperazine-1-yl)-acetyl]-Peg3-Peg3)-
AQRAFIEWLLAQGPSSGAPPPS;
(SEQ ID NO: 20)
Y-Aib-EGTFISDYSIELDK-K((19-Carboxy-nonadecanoyl)-
[(Piperazine-1-yl)-acetyl]-Peg3-Peg3)-
AQRAFVEWLLAQGPSSGAPPPS;
(SEQ ID NO: 21)
Y-Aib-EGTFISDYSIELDK-K((19-Carboxy-nonadecanoyl)-
[(Piperazine-1-yl)-acetyl]-Peg3-Peg3)-
AQKEFVEWLLAAGPSSGAPPPS;
(SEQ ID NO: 22)
Y-Aib-EGTFISDYSIELDK-K([19-Carboxy-nonadecanoyl]-
isoGlu-Peg3-Peg3)-AQKEFVEWLLAAGPSSGAPPPS;
(SEQ ID NO: 23)
Y-Aib-EGTFISDYSIELDKIAQRAFIEWLLAGPSSGAPPPS-
K([19-Carboxy-nonadecanoyl]-isoGlu-Peg3-Peg3);
(SEQ ID NO: 24)
Y-Aib-EGTFISDYSIELDKIAQKEFIEWLLAGPSSGAPPPS-
K([19-Carboxy-nonadecanoyl]-isoGlu-Peg3-Peg3);
(SEQ ID NO: 25)
Y-Aib-EGTFISDYSIELDKIAAQDFIEWLLAGPSSGAPPPS-
K([19-Carboxy-nonadecanoyl]-isoGlu-Peg3-Peg3);
(SEQ ID NO: 26)
Y-Aib-EGTFISDYSIELDKIAAQDFIEWLLAGPSSGAPPPS-
K((19-Carboxy-nonadecanoyl)-[(Piperazine-1-yl)-
acetyl]-Peg3-Peg3);
(SEQ ID NO: 27)
Y-Aib-EGTFISDYSIELDKIAAQDFVEWLLAGPSSGAPPPS-
K([19-Carboxy-nonadecanoyl]-isoGlu-Peg3-Peg3);
(SEQ ID NO: 28)
Y-Aib-EGTFISDYSIELDKIAQRAFIEWLLAQGPSSGAPPPS-
K([19-Carboxy-nonadecanoyl]-isoGlu-Peg3-Peg3);
(SEQ ID NO: 29)
Y-Aib-EGTFISDYSIELDK-K([19-Carboxy-nonadecanoyl]-
isoGlu-Peg3-Peg3)-AAQAFVNWLLAGPSSGAPPPS;
(SEQ ID NO: 30)
Y-Aib-EGTFISDYSIELDK-K([19-Carboxy-nonadecanoyl]-
isoGlu-Peg3-Peg3)-AAQDFVNWLLAAGPSSGAPPPS;
(SEQ ID NO: 31)
Y-Aib-EGTFISDYSIELDK-K([19-Carboxy-nonadecanoyl]-
isoGlu-Peg3-Peg3)-AAQDFINWLLAGPSSGAPPPS;
(SEQ ID NO: 32)
Y-Aib-EGTFISDYSIELDK-K([19-Carboxy-nonadecanoyl]-
isoGlu-Peg3-Peg3)-AAQDFIEWLLAGPSSGAPPPS;
(SEQ ID NO: 33)
Y-Aib-EGTFISDYSIELDK-K((19-Carboxy-nonadecanoyl)-
[(Piperazine-1-yl)-acetyl]-Peg3-Peg3)-
AAQDFIEWLLAGPSSGAPPPS;
(SEQ ID NO: 34)
Y-Aib-EGTFISDYSIELD-K([19-Carboxy-nonadecanoyl]-
isoGlu-Peg3-Peg3)-IAQRAFIEWLLAQGPSSGAPPPS;
(SEQ ID NO: 35)
Y-Aib-EGTFISDYS-K([19-Carboxy-nonadecanoyl]-
isoGlu-Peg3-Peg3)-ELDKIAQRAFIEWLLAQGPSSGAPPPS;
(SEQ ID NO: 36)
Y-DAla-EGTFISDYSIELDK-K([19-Carboxy-nonadecanoyl]-
isoGlu-Peg3-Peg3)-AQRAFIEWLLAQGPSSGAPPPS;
(SEQ ID NO: 37)
Y-DAla-EGTFISDYSIELDKIAAQDFIEWLLAGPSSGAPPPS-
K((19-Carboxy-nonadecanoyl)-[(Piperazine-1-yl)
-acetyl]-Peg3-Peg3);
(SEQ ID NO: 38)
Y-Aib-EGTFISDYSIELDK-K([19-Carboxy-nonadecanoyl]-
isoGlu-Peg3-Peg3)-AAQDFIEWLLAQGPSSGAPPPS;
(SEQ ID NO: 39)
Y-Aib-EGTFISDYSIELDK-K([19-Carboxy-nonadecanoyl]-
isoGlu-Peg3-Peg3)-AAQDFINWLLAQGPSSGAPPPS;
(SEQ ID NO: 40)
Y-Aib-EGTFISDYSIELDK-K([19-Carboxy-nonadecanoyl]-
isoGlu-Peg3-Peg3)-AAQAFIEWLLAQGPSSGAPPPS;
or
(SEQ ID NO: 41)
Y-Aib-EGTFISDYSIELDK-K((19-Carboxy-nonadecanoyl)-
[(Piperazine-1-yl)-acetyl]-Peg3-Peg3)-
AAQAFIEWLLAQGPSSGAPPPS.

The GIP-analogue may be

(SEQ ID NO: 89)
H-Y-Aib-EGTFISDYSIELDK-K(Hexadecanoyl-isoGlu)-
HQQDFVNWLLAQGPSSGAPPPS-NH₂;
(Compound 1)
(SEQ ID NO: 90)
H-Y-Aib-EGTFISDYSIELD-K(Hexadecanoyl-isoGlu)-
IHQQDFVNWLLAQGPSSGAPPPS-NH₂;
(Compound 2)
(SEQ ID NO: 91)
H-Y-Aib-EGTFISDYSIELEK-K(Hexadecanoyl-isoGlu)-
HQQDFVNWLLAQGPSSGAPPPS-NH₂;
(Compound 3)
(SEQ ID NO: 92)
H-Y-Aib-EGTFISDYSIELDKIHQQDFVNWLLAQGPSSGAPPPS-
K([19-carboxy-nonadecanoyl]-
isoGlu-Peg3-Peg3)-NH₂ (Compound 4);
(SEQ ID NO: 93)
H-Y-Aib-EGTFISDYSIELDKIHQQDFVNWLLAQGPSSGAPPPS-
K(Hexadecanoyl-isoGlu)-NH₂;
(Compound 5)
(SEQ ID NO: 94)
H-Y-Aib-EGTFISDYSIELDKIHQQDFVNWLLAQ-
K(Hexadecanoyl-isoGlu)-NH₂;
(Compound 6);
(SEQ ID NO: 95)
H-Y-Aib-EGTFISDYSIELDKIHQQDFVNWLLAQKG-
K(Hexadecanoyl-isoGlu)-NH₂;
(Compound 7)
(SEQ ID NO: 96)
H-Y-Aib-EGTFISDYSIELDK-K([19-carboxy-
nonadecanoyl]-isoGlu-Peg3-Peg3)-
HQQDFVNYLLAQGPSSGAPPPS-NH₂;
(Compound 8)
(SEQ ID NO: 97)
H-Y-Aib-EGTFISDYSIELDK-K([19-carboxy-
nonadecanoyl]-isoGlu-Peg3-Peg3)-
HQQDFVNWLLAQGPSSGAPPPS-NH₂;
(Compound 9)
(SEQ ID NO: 98)
H-Y-Aib-EGTFISDYSIELDK-K([19-carboxy-
nonadecanoyl]-isoGlu-Peg3-Peg3)-
AAQDFVNWLLAQGPSSGAPPPS-NH₂;
(Compound 10)
(SEQ ID NO: 99)
H-Y-Aib-EGTFISDYSIELEK-K([19-carboxy-
nonadecanoyl]-isoGlu-Peg3-Peg3)-
AAKEFVNWLLAQGPSSGAPPPS-NH₂;
(Compound 11)
(SEQ ID NO: 100)
H-Y-Aib-EGTFISDYSIELEK-K([19-carboxy-
nonadecanoyl]-isoGlu-Peg3-Peg3)-
AQRAFVEWLLAQGPSSGAPPPS-NH₂;
(Compound 12)
(SEQ ID NO: 101)
H-Y-Aib-EGTFISDYSIELEKIAQRAFVEWLLAQG
PSSGAPPPS-K([19-carboxy-nonadecanoyl]-
isoGlu-Peg3-Peg3)-NH₂;
(Compound 13)
(SEQ ID NO: 102)
H-Y-Aib-EGTFISDYSIELEKIAQRAFVEWLLAQ-
K([19-carboxy-nonadecanoyl]-isoGlu-Peg3-
Peg3)-NH₂;
(Compound 14)
(SEQ ID NO: 103)
H-Y-Aib-EGTFISDYSIELDK-K([19-carboxy-
nonadecanoyl]-isoGlu-Peg3-Peg3)-
AAQDFVNWLLAGPSSGAPPPS-NH₂;
(Compound 15)
(SEQ ID NO: 104)
H-Y-Aib-EGTFISDYSIELDKIAAQDFVNWLLAG
PSSGAPPPS-K([19-carboxy-nonadecanoyl]-
isoGlu-Peg3-Peg3)-NH₂;
(Compound 16)
(SEQ ID NO: 105)
H-Y-Aib-EGTFISDYSIELDK-K([19-Carboxy-
nonadecanoyl]-isoGlu-Peg3-Peg3)-
AQRAFVEWLLAQGPSSGAPPPS-NH₂;
(Compound 17)
(SEQ ID NO: 106)
H-Y-Aib-EGTFISDYSIELDK-K([19-Carboxy-
nonadecanoyl]-isoGlu-Peg3-Peg3)-
AQRAFIEWLLAQGPSSGAPPPS-NH₂;
(Compound 18)
(SEQ ID NO: 107)
H-Y-Aib-EGTFISDYSIELDK-K((19-Carboxy-
nonadecanoyl)-[(Piperazine-1-yl)-
acetyl]-Peg3-Peg3)-AQRAFIEWLLAQGP
SSGAPPPS-NH₂;
(Compound 19)
(SEQ ID NO: 108)
H-Y-Aib-EGTFISDYSIELDK-K((19-Carboxy-
nonadecanoyl)-[(Piperazine-1-yl)-acetyl]-
Peg3-Peg3)-AQRAFVEWLLAQGPSSGAPPPS-NH₂;
(Compound 20)
(SEQ ID NO: 109)
H-Y-Aib-EGTFISDYSIELDK-K((19-Carboxy-
nonadecanoyl)-[(Piperazine-1-yl)-acetyl]-
Peg3-Peg3)-AQKEFVEWLLAAGPSSGAPPPS-NH₂;
(Compound 21)
(SEQ ID NO: 110)
H-Y-Aib-EGTFISDYSIELDK-K([19-Carboxy-
nonadecanoyl]-isoGlu-Peg3-Peg3)-
AQKEFVEWLLAAGPSSGAPPPS-NH₂;
(Compound 22)
(SEQ ID NO: 111)
H-Y-Aib-EGTFISDYSIELDKIAQRAFIEWLLAGP
SSGAPPPS-K([19-Carboxy-nonadecanoyl]-
isoGlu-Peg3-Peg3)-NH₂;
(Compound 23)
(SEQ ID NO: 112)
H-Y-Aib-EGTFISDYSIELDKIAQKEFIEWLLAGP
SSGAPPPS-K([19-Carboxy-nonadecanoyl]-
isoGlu-Peg3-Peg3)-NH₂;
(Compound 24)
(SEQ ID NO: 113)
H-Y-Aib-EGTFISDYSIELDKIAAQDFIEWLLAGP
SSGAPPPS-K([19-Carboxy-nonadecanoyl]-
isoGlu-Peg3-Peg3)-NH₂;
(Compound 25)
(SEQ ID NO: 114)
H-Y-Aib-EGTFISDYSIELDKIAAQDFIEWLLAGP
SSGAPPPS-K((19-Carboxy-nonadecanoyl)-
[(Piperazine-1-yl)-acetyl]-Peg3-Peg3)-NH₂;
(Compound 26)
(SEQ ID NO: 115)
H-Y-Aib-EGTFISDYSIELDKIAAQDFVEWLLAGPSS
GAPPPS-K([19-Carboxy-nonadecanoyl]-
isoGlu-Peg3-Peg3)-NH₂;
(Compound 27)
(SEQ ID NO: 116)
H-Y-Aib-EGTFISDYSIELDKIAQRAFIEWLLAQGPS
SGAPPPS-K([19-Carboxy-nonadecanoyl]-
isoGlu-Peg3-Peg3)-NH₂;
(Compound 28)
(SEQ ID NO: 117)
H-Y-Aib-EGTFISDYSIELDK-K([19-Carboxy-
nonadecanoyl]-isoGlu-Peg3-Peg3)-
AAQAFVNWLLAGPSSGAPPPS-NH₂;
(Compound 29)
(SEQ ID NO: 118)
H-Y-Aib-EGTFISDYSIELDK-K([19-Carboxy-
nonadecanoyl]-isoGlu-Peg3-Peg3)-
AAQDFVNWLLAAGPSSGAPPPS-NH₂;
(Compound 30)
(SEQ ID NO: 119)
H-Y-Aib-EGTFISDYSIELDK-K([19-Carboxy-
nonadecanoyl]-isoGlu-Peg3-Peg3)-
AAQDFINWLLAGPSSGAPPPS-NH₂;
(Compound 31)
(SEQ ID NO: 120)
H-Y-Aib-EGTFISDYSIELDK-K([19-Carboxy-
nonadecanoyl]-isoGlu-Peg3-Peg3)-
AAQDFIEWLLAGPSSGAPPPS-NH₂;
(Compound 32)
(SEQ ID NO: 121)
H-Y-Aib-EGTFISDYSIELDK-K((19-Carboxy-
nonadecanoyl)-[(Piperazine-1-yl)-acetyl]-
Peg3-Peg3)-AAQDFIEWLLAGPSSGAPPPS-NH₂;
(Compound 33)
(SEQ ID NO: 122)
H-Y-Aib-EGTFISDYSIELD-K([19-Carboxy-
nonadecanoyl]-isoGlu-Peg3-Peg3)-
IAQRAFIEWLLAQGPSSGAPPPS-NH₂;
(Compound 34)
(SEQ ID NO: 123)
H-Y-Aib-EGTFISDYS-K([19-Carboxy-
nonadecanoyl]-isoGlu-Peg3-Peg3)-
ELDKIAQRAFIEWLLAQGPSSGAPPPS-NH₂;;
(Compound 35)
(SEQ ID NO: 124)
H-Y-DAlA-EGTFISDYSIELDK-K([19-Carboxy-
nonadecanoyl]-isoGlu-Peg3-Peg3)-
AQRAFIEWLLAQGPSSGAPPPS-NH₂;
(Compound 36)
(SEQ ID NO: 125)
H-Y-DAlA-EGTFISDYSIELDKIAAQDFIEWLLAG
PSSGAPPPS-K((19-Carboxy-nonadecanoyl)-
[(Piperazine-1-yl)-acetyl]-Peg3-Peg3)-NH₂;
(Compound 37)
(SEQ ID NO: 126)
H-Y-Aib-EGTFISDYSIELDK-K([19-Carboxy-
nonadecanoyl]-isoGlu-Peg3-Peg3)-
AAQDFIEWLLAQGPSSGAPPPS-NH₂;
(Compound 38)
(SEQ ID NO: 127)
H-Y-Aib-EGTFISDYSIELDK-K([19-Carboxy-
nonadecanoyl]-isoGlu-Peg3-Peg3)-
AAQDFINWLLAQGPSSGAPPPS-NH₂;
(Compound 39)
(SEQ ID NO: 128)
H-Y-Aib-EGTFISDYSIELDK-K([19-Carboxy-
nonadecanoyl]-isoGlu-Peg3-Peg3)-
AAQAFIEWLLAQGPSSGAPPPS-NH₂;
(Compound 40)
and
or
(SEQ ID NO: 129)
H-Y-Aib-EGTFISDYSIELDK-K((19-Carboxy-
nonadecanoyl)-[(Piperazine-1-yl)-acetyl]-
Peg3-Peg3)-AAQAFIEWLLAQGPSSGAPPPS-NH₂.
(Compound 41)

The invention further provides a pharmaceutical composition comprising a GIP analogue as described herein, or a pharmaceutically acceptable salt or solvate thereof, in admixture with a carrier, preferably a pharmaceutically acceptable carrier. The GIP analogue may, for example, be a pharmaceutically acceptable acid addition salt.

The pharmaceutical composition may be formulated as a liquid suitable for administration by injection or infusion. The pharmaceutical composition may be formulated to cause controlled, e.g., slow release of said GIP analogue.

The invention further provides a therapeutic kit comprising a GIP analogue as described herein, and a device comprising a GIP analogue as described herein.

The invention further provides a GIP analogue as described herein, or a pharmaceutically acceptable salt or solvate thereof, for use in a method of medical treatment, e.g. for use in the treatment and/or prevention of a metabolic disorder.

The invention further provides the use of a GIP analogue as described herein, or a pharmaceutically acceptable salt or solvate thereof, in the preparation of a medicament for the treatment and/or prevention of a metabolic disorder.

The invention further provides a method of prevention and or/treatment of a metabolic disorder in a subject, comprising administering a GIP analogue as described herein, or a pharmaceutically acceptable salt or solvate thereof, to the subject.

The metabolic disorder may be diabetes or a diabetes related disorder, or obesity or an obesity related disorder. The link between obesity and diabetes is well known, so these conditions may be but are not necessarily separate or mutually exclusive.

Diabetes related disorders include insulin resistance, glucose intolerance, increased fasting glucose, pre-diabetes, type 1 diabetes, type 2 diabetes, gestational diabetes hypertension, dyslipidemia, and combinations thereof.

Diabetes related disorders also include atherosclerosis, arteriosclerosis, coronary heart disease, peripheral artery disease and stroke; or conditions associated with atherogenic dyslipidemia, blood fat disorders, elevated blood pressure, hypertension, a prothrombotic state, a proinflammatory state, and bone related disorders such as osteoporosis.

The blood fat disorder may be selected from high triglycerides, low HDL cholesterol, high LDL cholesterol, and plaque buildup in artery walls, or a combination thereof.

The prothrombotic state may be selected from high fibrinogen levels in the blood and high plasminogen activator inhibitor-1 levels in the blood.

The proinflammatory state may be an elevated C-reactive protein level in the blood.

Obesity related disorders include obesity linked inflammation, obesity linked gallbladder disease and obesity induced sleep apnea, or may be associated with a condition selected from atherogenic dyslipidemia, blood fat disorders, elevated blood pressure, hypertension, a prothrombotic state, and a proinflammatory state, or a combination thereof.

### BRIEF DESCRIPTION OF THE DRAWINGS

FIGS. 1A-1D: Blood glucose levels (1A-1C) and area under the blood glucose curves (AUC) (1D) in an OGTT in 5-hour fasted mice. The mice were injected s.c. with vehicle, the GLP-1 analogue liraglutide (10 nmol/kg), and GIP receptor agonists (compound 12, 13, 17, and 21 at 3-300 nmol/kg) 4 hours prior to the oral gavage of glucose (t=0). Data are means±SEM; n=6. Statistical differences vs vehicle: *p<0.05, **p<0.01, ***p<0.001.

FIGS. 2A-2E: Blood glucose levels (2A-2D) and area under the blood glucose curves (AUC) (2E) in an OGTT in 5-hour fasted mice. The mice were injected s.c. with vehicle and GIP receptor agonists (compounds 12, 18, 41, 33, and 35 at 3-300 nmol/kg) 4 hours prior to the oral gavage of glucose (t=0). Data are means +SEM; n=6. Statistical differences vs vehicle: ***p<0.001.

FIG. 3: Relative body weight changes (delta Δ bodyweight=body weight at each study day−body weight at day 1) in DIO mice during three weeks of treatment. Animals were treated once daily with two separate s.c. injections. The first injection was with vehicle 1 or GLP-1 analogue liraglutide (20 nmol/kg). The second injection was with vehicle 2 or Compound 12 (3 and 30 nmol/kg). The GIP agonist was only dosed every third day of the study (starting on day 1). On other days, GIP agonist was replaced with vehicle 2. Data are means±SEM; n=8-9. Statistical differences vs vehicle on day 22: ***p<0.001. Statistical difference (p<0.05) between liraglutide and liraglutide co-treated GIP agonist is shown with a line.

FIGS. 4A-4E: Relative body weight changes (delta Δ body weight=body weight at each study day−body weight at day 0) in DIO mice during four weeks of treatment with vehicle, GLP-1 analogue liraglutide, liraglutide+Compound 10 or 12 (FIG. 4A), liraglutide+Compound 17 (FIG. 4B), liraglutide+Compound 18 (FIG. 4C), liraglutide+compound 35 (FIG. 4D) or liraglutide+Compound 41 (FIG. 4E). Animals were treated once daily with two separate s.c. injections. The first injection was with vehicle 1 or liraglutide (20 nmol/kg). The second injection was with vehicle 2 or GIP agonists (30 and/or 300 nmol/kg). The GIP agonists were only dosed every third day of the study (starting on day 0). On other days, GIP agonists were replaced with vehicle 2. Data are means±SEM; n=9. Statistical differences vs vehicle on day 27: ***p<0.001. Statistical differences (p<0.05) between liraglutide and liraglutide co-treated with GIP agonist are shown with lines.

### DETAILED DESCRIPTION OF THE INVENTION

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well known and commonly used in the art.

### Definitions

Unless specified otherwise, the following definitions are provided for specific terms, which are used in the above written description.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The terms “patient,” “subject,” and “individual” may be used interchangeably and refer to either a human or a non-human animal. These terms include mammals such as humans, primates, livestock animals (e.g., bovines, porcines), companion animals (e.g., canines, felines) and rodents (e.g., mice and rats).

The term “solvate” in the context of the present invention refers to a complex of defined stoichiometry formed between a solute (in casu, a peptide conjugate or pharmaceutically acceptable salt thereof according to the invention) and a solvent. The solvent in this connection may, for example, be water, ethanol or another pharmaceutically acceptable, typically small-molecular organic species, such as, but not limited to, acetic acid or lactic acid. When the solvent in question is water, such a solvate is normally referred to as a hydrate.

The term “agonist” as employed in the context of the invention refers to a substance (ligand) that activates signaling by the receptor type in question. The term “antagonist” as employed in the context of the invention refers to a substance (ligand) that decreases signaling by the receptor type in question.

Throughout the description and claims the conventional one-letter and three-letter codes for natural (or “proteinogenic”) amino acids are used, as well as generally accepted three letter codes for other (non-natural or “non-proteinogenic”) α-amino acids, such as Aib (α-aminoisobutyric acid), Orn (ornithine) and D-Ala (D-alanine). All amino acid residues in peptides of the invention are preferably of the L-configuration except where explicitly stated.

Among sequences disclosed herein are sequences incorporating an “H—” moiety at the amino terminus (N-terminus) of the sequence, and either an “—OH” moiety or an “—NH₂” moiety at the carboxy terminus (C-terminus) of the sequence. In such cases, and unless otherwise indicated, an “H—” moiety at the N-terminus of the sequence in question indicates a hydrogen atom (i.e. R¹=H), corresponding to the presence of a free primary or secondary amino group at the N-terminus, while an “—OH” or an “—NH₂” moiety at the C-terminus of the sequence (i.e. R²=OH or NH₂) indicates a carboxy (COOH) group or an amido (CONH₂) group at the C-terminus, respectively.

The compounds of the present invention have GIP biological activity, in particular in treatment of metabolic diseases such as diabetes and obesity. This can be assessed, e.g., in in vivo assays, in which the blood glucose level or another biological activity is determined after a test animal has been treated or exposed to a GIP analogue. The compounds of the present invention may be particular effective in improving glycaemic control and reducing body weight when administered together with a GLP-1 receptor agonist to a diabetic patient and/or an overweight or obese subject. The effect obtained with this combination therapy may be superior to that obtained with the administration of a GLP-1 receptor agonist alone in comparable subjects when given according to a comparable dosing regime. The compounds of the present invention may also be capable of improving glycaemic control and reducing bodyweight when administered alone. The Y1 and Y2 group has a stabilizing effect on the GIP analogues. Without being bound to any theory it is believed that group comprising the C-terminus part of exendin-4 and GIP compounds has impact on the folding of the peptide.

In either the treatment of a diabetic subject or an overweight subject, the effect of treating with a GIP analogue of the present invention may be superior to that obtained with an equivalent quantity (by mass, or molar ratio) of wild type human GIP in comparable subjects when given according to a comparable dosing regime, alone or in combination with another anti-diabetic or anti-obesity agent.

Activity in in vitro assays may also be used as a measure of the compounds' activity. Typically the compounds have activity (i.e. agonist activity) at the GIP receptor (designated GIP-R). EC₅₀ values may be used as a numerical measure of agonist potency at a given receptor. An EC₅₀ value is a measure of the concentration of a compound required to achieve half of that compound's maximal activity in a particular assay. In any given assay, the EC₅₀ value of a compound in a given assay may be assessed relative to the EC₅₀ of human GIP. Thus, the ratio of the EC₅₀ value of the test compound to the EC₅₀ value of wild type human GIP (EC₅₀[test compound]/EC₅₀[GIP]) at the human GIP receptor may be less than 10, less than 5, less than 1, less than 0.1, less than 0.05 or less than 0.01. EC₅₀ values may be determined using the human GIP receptor assay described in the Examples below. In such an assay, the compounds may, for example, have an EC₅₀ value of 0.001-0.050 nM, 0.001-0.030 nM, 0.001-0.020 nM, or 0.001-0.010 nM.

The compounds typically have minimal or no agonist activity at the GLP-1 receptor. For example, the ratio of the EC₅₀ value of the test compound to the EC₅₀ value of the GLP-1 agonist Exendin-4 (EC₅₀[test compound]/EC₅₀[Ex4]) at the human GIP receptor may be at least about 100, at least about 250, at least about 500, at least about 750, at least about 1000, at least about 5000, or at least about 10,000. (“About” is used here to signify +/−10%.) EC₅₀ values may be determined using the human GLP-1 receptor assay described in the Examples below. In such an assay, the compounds may, for example, have an EC₅₀ value of at least 1 nM, at least 3 nM, at least 5 nM or at least 10 nM.

### Lipophilic group

The compound of the invention comprises a residue Ψ, i.e. a residue selected from Lys, Arg, Orn and Cys in which the side chain is conjugated to a lipophilic substituent.

Without wishing to be bound by any particular theory, it is thought that the substituent binds plasma proteins (e.g. albumin) in the blood stream, thus shielding the compounds of the invention from enzymatic degradation and thereby enhancing the half-life of the compounds. It may also modulate the potency of the compound, e.g. with respect to the GIP receptor.

The substituent is conjugated to the functional group at the distal end of the side chain from the alpha-carbon. The normal ability of the Lys, Arg, Orn or Cys side chain to participate in interactions mediated by that functional group (e.g. intra- and inter-molecular interactions) may therefore be reduced or completely eliminated by the presence of the substituent. Thus, the overall properties of the compound may be relatively insensitive to changes in the actual amino acid present as residue Ψ. Consequently, it is believed that any of the residues Lys, Arg, Orn and Cys may be present at any position where Y is permitted. However, in certain embodiments, it may be advantageous that the amino acid component of Ψ is Lys.

Thus, Ψ is a residue of Lys, Arg, Orn or Cys in which the side chain is conjugated to a substituent having the formula —Z¹ or —Z²—Z¹.
—Z¹ is a fatty chain having at a terminus a connection —X— to Ψ or to Z²;
wherein
—X— is a bond, —CO—, —SO—, or —SO₂—;
and, optionally, Z¹ has a polar group at the end of the chain distal from connection —X—; said
polar group comprising a carboxylic acid or a carboxylic acid bioisostere, a phosphonic acid, or a sulfonic acid group;
and wherein —Z²—, if present, is a spacer of formula:

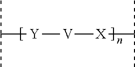

connecting Z¹ to Ψ;
wherein:
each Y is independently —NH, —NR, —S or —O, where R is alkyl, a protecting group or forms a linkage to another part of the spacer Z²;
each X is independently a bond, CO—, SO—, or SO₂—;
with the proviso that when Y is —S, the X to which it is bound is a bond;
each V is independently a bivalent organic moiety linking Y and X;
and n is 1-10.

### The Group Z¹

Z¹ is a fatty chain having a connection to Ψ or to Z², referred to herein as —X—. —X— may be, for example, a bond, acyl (—CO—), sulfinyl (—SO—), or sulfonyl (—SO₂—). When Z¹ is bound directly to Ψ, that is, when Z² is not present, preferably —X— is acyl (—CO—), sulfinyl (—SO—), or sulfonyl (—SO₂—). Most preferably, —X— is acyl (—CO—).

Z¹ may further have a polar group, said polar group being located at the end of the chain distal from the connection —X—. In other words, the connection is located at the ω-position with respect to the polar group. The polar group may be bound directly to the terminus of the fatty chain, or may be bound via a linker.

Preferably, the polar group is an acidic or weakly acid group, for example a carboxylic acid or a carboxylic acid bioisostere, a phosphonate, or a sulfonate. The polar group may have a pKₐ of between −2 and 12 in water, more preferably between 1 and 7, more preferably between 3 and 6. Certain preferred polar groups have a pKₐ of between 4 and 5.

For example, and not by way of limitation, the polar group may comprise a carboxylic acid (—COOH) or a carboxylic acid bioisostere, a phosphonic acid (—P(O)(OH)₂), or a sulfonic acid (—SO₂OH) group.

Preferably the polar group, if present, comprises a carboxylic acid or carboxylic acid bioisostere. Suitable carboxylic acid bioisosteres are known in the art. Preferably the bioisostere has a proton having a pKₐ similar to the corresponding carboxylic acid. Examples of suitable bioisoteres may include, not by way of limitation, tetrazole, acylsulfomides, acylhydroxylamine, and squaric acid derivatives, as shown below ( - - - indicates the point of attachment):

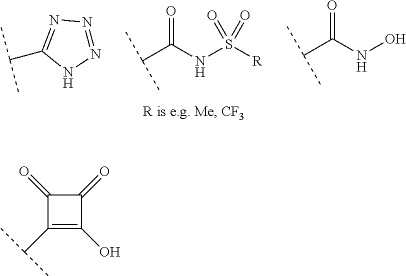

Fatty chain as used herein refers to a moiety comprising a chain of carbon atoms, the carbon atoms being predominantly substituted with hydrogen or hydrogen-like atoms, for example, a hydrocarbon chain. Such fatty chains are often referred to as lipophilic, although it will be appreciated that substitution may alter the lipophilic properties of the overall molecule.

The fatty chain may by aliphatic. It may be entirely saturated or may include one or more double or triple bonds. Each double bond, if present, may be in the E or Z configuration. The fatty chain may also have one or more cycloalkylene or heterocycloalkylene moieties in its length, and additionally or alternatively may have one or more arylene or heteroarylene moieties in its length. For example, the fatty chain may incorporate a phenylene or piperazinylene moiety in its length as, for example, shown below (wherein - - - represents the points of attachment within the chain).

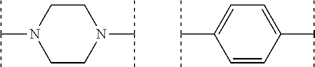

The fatty chain may be derived from a fatty acid, for example, it may be derived from a medium-chain fatty acid (MCFA) with an aliphatic tail of 6-12 carbon atoms, a long-chain fatty acid (LCFA) with an aliphatic tail of 13-21 carbon atoms, or a very long-chain fatty acid (LCFA) with an aliphatic tail of 22 carbon atoms or more. Examples of linear saturated fatty acids from which suitable fatty chains may be derived include tridecylic (tridecanoic) acid, myristic (tetradecanoic) acid, pentadecylic (pentadecanoic) acid, palmitic (hexadecanoic) acid, and margaric (heptadecanoic) acid. Examples of linear unsaturated fatty acids from which suitable fatty chains may be derived include myristoleic acid, palmitoleic acid, sapienic acid and oleic acid.

The fatty chain may be connected to Ψ or to Z² by an amide linkage, a sulfinamide linkage, a sulfonamide linkage, or by an ester linkage, or by an ether, thioether or amine linkage. Accordingly, the fatty chain may have, a bond to Ψ or to Z² or an acyl (—CO—), sulfinyl (—SO—), or sulfonyl (—SO₂—) group. Preferably, the fatty chain has a terminus having an acyl (—CO—) group and is connected to Ψ or Z² by an amide or ester linkage.

In some embodiments, Z¹ is a group of formula:

A-B-Alk-X—
wherein
A is hydrogen or a carboxylic acid, a carboxylic acid bioisostere, a phosphonic acid, or a sulfonic acid group;
B is a bond or a linker;
X is a bond, acyl (—CO—), sulfinyl (—SO—), or sulfonyl (—SO₂—); and
Alk is a fatty chain that may be optionally substituted with one or more substituents. The fatty chain is preferably 6 to 28 carbon atoms in length (e.g. a C₆₋₂₈alkylene), more preferably, 12 to 26 carbons in length (e.g. a C₁₂₋₂₆alkylene), more preferably, 16 to 22 carbons in length (e.g. C₁₆₋₂₂alkylene), and may be saturated or unsaturated. Preferably, Alk is saturated, that is, preferably Alk is alkylene.

Optional substituents on the fatty chain may be independently selected from fluoro, C₁₋₄alkyl, preferably methyl; trifluoromethyl, hydroxymethyl, amino, hydroxyl, C₁₋₄alkoxy, preferably methoxy; oxo, and carboxyl, and may be independently located at any point along the chain. In some embodiments, each optional substituent is selected from fluoro, methyl, and hydroxyl. Where more than one substituent is present, substituents may be the same or different. Preferably, the number of substituents is 0 to 3; more preferably the fatty chain is unsubstituted.

B may be a bond or a linker. When B is a linker, it may be a cycloalkylene, heterocycloalkylene, C₆arylene, or C₅₋₆heteroarylene, or C₆arylene-O— or C₅₋₆heteroarylene-O—.

When B is phenylene it may, for example, be selected from 1,2-phenylene, 1,3-phenylene, 1,4-phenylene, preferably 1,4-phenylene (so that A-B- is a 4-benzoic acid substituent or 4-benzoic acid bioisostere). When B is phenylene-O—, it may, for example, be selected from 1,2-phenylene-O—, 1,3-phenylene-O—, 1,4-phenylene-O—, preferably 1,4-phenylene-O. Each phenylene of B may be optionally substituted with one or more substituents selected from fluoro, methyl, trifluoromethyl, amino, hydroxyl, and C₁₋₄alkoxy, preferably methoxy. It will be appreciated that substituent identity and position may be selected to subtly alter the pKₐ of the polar group. Suitable inductively or mesomerically electron-withdrawing or donating groups and their positional effects are known in the art. In some embodiments, B may be C₅₋₆heteroarylene, for example, pyridinylene or thiofuranylene, and may be optionally substituted as described.

For example, in some embodiments, A-B- may be selected from:

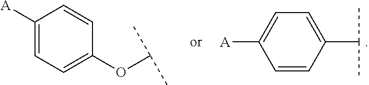

Preferably, A is H— or HOOC— and B is a bond.

It will be understood that when A is hydrogen, B is a bond and Alk is unsubstituted alkylene, A-B-Alk- is an alkyl chain of formula H₃C—(CH₂)ₙ—.

In some embodiments, Z¹ is an acyl group of formula:

A-B-Alk-(CO)—
or a sulfonyl group of formula:

A-B-Alk-(SO₂)—.

Preferably, Z¹ is an acyl group of formula:

A-B-alkylene-(CO)—
where A and B are as defined above.

In some embodiments, A is —COOH and B is a bond. Accordingly, certain preferred Z¹ are derived from long-chain saturated α,ω-dicarboxylic acids of formula HOOC—(CH₂)₁₂₋₂₂—COOH, preferably, long-chain saturated α,ω-dicarboxylic acids having an even number of carbon atoms in the aliphatic chain. In some other embodiments, A is H and B is a bond. Accordingly, certain preferred Z¹ are derived from long-chain saturated carboxylic acids of formula HOOC—(CH₂)₁₂₋₂₂—CH₃, preferably, long-chain saturated carboxylic acids having an even number of carbon atoms in the aliphatic chain.

For example, and not by way of limitation, Z¹ may be:
A-B-C₁₆₋₂₀alkylene-(CO)— wherein A is H or —COOH and B is a bond, for example:
17-carboxy-heptadecanoyl HOOC—(CH₂)₁₆—(CO)—;
19-carboxy-nonadecanoyl HOOC—(CH₂)₁₈—(CO)—;
Octadecanoyl H₃C—(CH₂)₁₆—(CO)—;
Eicosanoyl H₃C—(CH₂)₁₈—(CO)—;

The carboxylic acid group, if present, may be replaced by a bioisotere as detailed herein.

### The Group Z²

Z² is an optional spacer that connects Z¹ to the side chain of the amino acid component of Ψ. At its most general, Z², if present, is a spacer bound at one terminus by Y, which may be a nitrogen, oxygen or sulfur atom, and at the other terminus by X, which may be a bond or an acyl (—CO—), sulfinyl (—SO—), sulfonyl (—SO₂—)or absent. Accordingly, Z² may be a spacer of formula ( - - - indicate points of attachment):

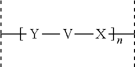

wherein:
Y may be —NH, —NR, —S or —O, where R may be alkyl, a protecting group or may form a linkage to another part of the spacer, with the remaining valency forming a linkage to Z¹;
X may be a bond, CO—, SO—, or SO₂—, with the remaining valency forming a linkage to the side chain of the amino acid component of Ψ;
V is a bivalent organic moiety linking Y and X;
and n may be 1, 2, 3, 4, 5, 6, 7, 8, 9 or 10. Where n is 2 or more, each Y, V, and X is independent of every other Y, V, and X.

Accordingly, Z² may be bound at each side by amide, sulfinamide, sulfonamide, or ester linkages or by amino, ether, or thioether linkages depending upon the nature of Y and X and the corresponding linking groups on Z¹ and the side chain. Where n is 2 or greater, each V may also be bound to each adjacent V by linkages as described. Preferably, linkages are amides, esters or sulfonamides, most preferably amides. Accordingly, in some embodiments, each Y is —NH or —NR and each X is CO— or SO₂—. Most preferably, —X— is acyl (—CO—).

In some embodiments, Z² is a spacer of formula —S_{A}—, —S_{B}—, —S_{A}—S_{B}— or —S_{B}—S_{A}—, wherein S_{A} and S_{B} are as defined below.

In some embodiments, Z² is selected from —S_{A}— or —S_{B}—S_{A}—, that is, [side chain]-Z²Z¹ is [side chain]-S_{A}—Z¹ or [side chain]-S_{B}—S_{A}—Z¹.

### The Group S_{A}

S_{A} may be a single amino acid residue or a residue of an amino acid derivative, especially an amino acid derivative residue having a sulfinyl or sulfonyl in place of the carboxy moiety at the C terminus. Additionally or alternatively, the single amino acid residue may have an oxygen or sulfur atom in place of the nitrogen atom at the N terminus.

S_{A} may be or may comprise a nitrogen-containing heterocycle, said nitrogen-containing heterocycle being bound within the lipophilic group at one end via a bond, a carboxy, a sulfinyl, or a sulfonyl group and at the other via a ring nitrogen atom. For example, S_{A} may comprise a piperazine ring.

Suitably, S_{A} is a 5-8-membered heterocycle having 1 or 2 nitrogen atoms and substituted with an X group, where X is a bond, CO—, SO—, or SO₂—, and where L, if present, is C₁₋₄alkylene (-denotes a point of attachment within the lipophilic group).

Preferably, S_{A} is a 6-membered heterocycle having 1 or 2 nitrogen atoms, preferably 2, and substituted with a —CH₂CO—, —CH₂SO—, or —CH₂SO₂— group.

For example, S_{A} may be:

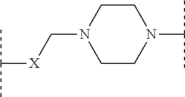

For example, S_{A} may be:

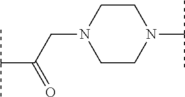

(referred to herein as piperazine-1-yl-acetyl).

Preferably, S_{A} is a single amino acid residue or piperazine-1-yl-acetyl. More preferably S_{A} is a single amino acid residue.

In some embodiments, the amino acid may be selected from γ-Glu, α-Glu, α-Asp, β-Asp, Ala, β-Ala (3-aminopropanoic acid), Dapa (2,3-diaminopropanoic acid), Dab (2,4-diaminobutanoic acid), and Gaba (4-aminobutanoic acid). It will be understood that where more than one carboxylic acid or amino moiety is present, connection may be at any moiety as appropriate. Any carboxylic acid or amino resides not bound within the residue may be free, that is, present as a free carboxylic acid or primary amine, or may be derivatised. Suitable derivatisation is known in the art. For example, carboxylic acid moieties may be present in S_{A} amino acid residues as esters, for example, as methyl esters. Amino moieties may be present as alkylated amines, for example, methylated, or may be protected as amide or carbamate moieties. Other suitable amino acids include β-Ala (3-aminopropanoic acid) and Gaba (4-aminobutanoic acid) and similar w amino acids.

It will be understood that amino acids may be D or L, or a racemic or enantioenriched mixture. In some embodiments, the amino acid is an L-amino acid. In some embodiments, the amino acid is a D-amino acid.

In some preferred embodiments, S_{A} has a carboxylic acid substituent, with γ-Glu, α-Glu, α-Asp, and β-Asp, and sulfinyl and sulfonyl derivatives thereof, being preferred. Accordingly, in some embodiments, the amino acid residue is:

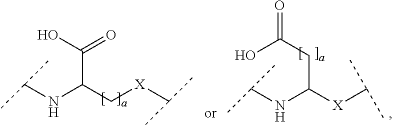

where —X— is —CO—, —SO—, —SO₂—, preferably —CO—, and a is 1 or 2, preferably 2. In some embodiments, the carboxylic acid is an ester, and the amino acid residue is:

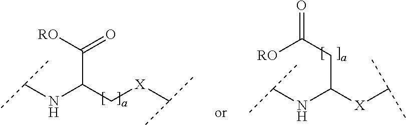

where —X— is —CO—, —SO—, —SO₂—, preferably —CO—, and a is 1 or 2, preferably 2, and R is C₁₋₄alkyl or C₆aryl. Preferably R is C₁₋₄alkyl, preferably methyl or ethyl, more preferably ethyl.

A preferred S_{A} group bearing a carboxylic acid is γ-Glu.

Preferably, S_{A} is selected from Dapa or γ-Glu. Most preferably, S_{A} is γ-Glu.

### The Group S_{B}

S_{B} may be a linker of general formula:

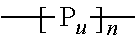

wherein P_{U} is a polymeric unit and n is 1, 2, 3, 4, 5, 6, 7, 8, 9 or 10. One terminus of the linker S_{B} is an —NH, —NR, —S or —O, wherein R may be alkyl, a protecting group or may form a linkage to another part of the polymeric unit; while the other is a bond or CO—, SO— or SO₂—. Accordingly, each polymeric unit P_{U} may be bound at each side by amide, sulfinamide, sulfonamide, or ester linkages or by amino, ether, or thioether linkages depending upon the nature of Y and X and the corresponding linking groups on Z¹, S_{A}, and Lys.

In some embodiments, each Pu may be independently a unit of formula:

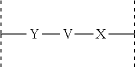

wherein:
Y may be —NH, —NR, —S or —O, wherein R may be alkyl, a protecting group or may form a linkage to another part of the spacer, with the remaining valency forming a linkage to Z¹;
X may be a bond, CO—, SO—, or SO₂—, with the remaining valency forming a linkage to the Ψ side chain;
and V is a bivalent organic moiety linking Y and X.

In some embodiments, V is the α-carbon of a natural or unnatural amino acid, that is V is —CHR^{AA}—, wherein R^{AA} is an amino acid side chain; or V is an optionally substituted C₁₋₆alkylene, or V is a chain comprising one or more units of ethylene glycol in series, also known as PEG chain, for example, —CH₂CH₂—(OCH₂CH₂)ₘ—O—(CH₂)ₚ—, where m is 0, 1, 2, 3, 4, or 5, and p is 1, 2, 3, 4, or 5; when X is CO—, p is preferably 1, 3, 4, or 5. Optional alkylene substituents include fluoro, methyl, hydroxy, hydroxymethy, and amino.

### Preferred P_{U} Units Include:

(i). Single amino acid residues: P_{U}ⁱ;
(ii). Dipeptide residues: P_{U}ⁱⁱ; and
(iii). Amino-(PEG)ₘ-carboxylic acid residues: P_{U}ⁱⁱⁱ,
   and may be present in any combination or order. For example, S_{B} may comprise one or more of each of P_{U}ⁱ, P_{U}ⁱⁱ, and P_{U}ⁱⁱⁱ in any order, or may comprise one or more units of P_{U}ⁱ, P_{U}ⁱⁱ, and P_{U}ⁱⁱⁱ only, or one of more units selected from P_{U}ⁱ and P_{U}ⁱⁱ, P_{U}ⁱ and P_{U}ⁱⁱⁱ, or P_{U}ⁱ and P_{U}ⁱⁱⁱ.
   (i). P_{U}ⁱ Single Amino Acid Residues

Each P_{U}ⁱ may be independently selected from any natural or unnatural amino acid residue and, for example, may be selected from Gly, Pro, Ala, Val, Leu, Ile, Met, Cys, Phe, Tyr, Trp, His, Lys, Arg, Gln, Asn, α-Glu, γ-Glu, Asp, Ser Thr, Dapa, Gaba, Aib, β-Ala, 5-aminopentanoyl, 6-aminohexanoyl, 7-aminoheptanoyl, 8-aminooctanoyl, 9-aminononanoyl, and 10-aminodecanoyl. Preferably, P_{U}ⁱ amino acid residues are selected from Gly, Ser, Ala, Thr, and Cys, more preferably from Gly and Ser.

In some embodiments, S_{B} is -(P_{U}ⁱ)ₙ-, wherein n is 1 to 8, more preferably 5 to 7, most preferably 6. In some preferred embodiments, S_{B} is -(P_{U}ⁱ)ₙ-, n is 6 and each P_{U}ⁱ is independently selected from Gly or Ser, with a preferred sequence being -Gly-Ser-Gly-Ser-Gly-Gly- (SEQ ID NO: 85).

(ii). P_{U}ⁱⁱ Dipeptide Residues

Each P_{U}ⁱⁱ may be independently selected from any dipeptide residue comprising two natural or unnatural amino acid residues bound by an amide linkage. Preferred P_{U}ⁱⁱ dipeptide residues include Gly-Gly, Gly-Ser, Ser-Gly, Gly-Ala, Ala-Gly, and Ala-Ala, more preferably Gly-Ser and Gly-Gly.

In some embodiments, S_{B} is -(P_{U}ⁱⁱ)ₙ-, wherein n is 2 to 4, more preferably 3, and each P_{U}ⁱⁱ is independently selected from Gly-Ser and Gly-Gly. In some preferred embodiments S_{B} is -(P_{U}ⁱⁱ)ₙ-, n is 3 and each P_{U}ⁱⁱ is independently selected from Gly-Ser and Gly-Gly, with a preferred sequence being -(Gly-Ser)-(Gly-Ser)-(Gly-Gly) (SEQ ID NO: 85).

Amino acids having stereogenic centres within P_{U}ⁱ and P_{U}ⁱⁱ may be racemic, enantioenriched, or enantiopure. In some embodiments, the or each amino acid is independently an L-amino acid. In some embodiments, the or each amino acid is independently a D-amino acid.

(iii). P_{U}ⁱⁱⁱ Amino-(PEG)ₘ-Carboxylic Acid Residues

Each P_{U}ⁱⁱⁱ may be independently a residue of general formula:

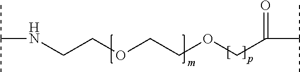

wherein m is 0, 1, 2, 3, 4, or 5, preferably 1 or 2, and p is 1, 3, 4, or 5, preferably 1.

In some embodiments, m is 1 and p is 1, that is, P_{U}ⁱⁱⁱ is a residue of 8-amino-3,6-dioxaoctanoic acid (also known as {2-[2-aminoethoxy]ethoxy}acetic acid and H₂N-PEG₃-COOH). This residue is referred to herein as —PEG₃—.

Other, longer, PEG chains are also known in the art. For example, 11-amino-3,6,9-trioxaundecanoic acid (also known as H₂N-PEG₄-COOH or -PEG₄-).

In some embodiments, S_{B} is -(P_{U}ⁱⁱⁱ)ₙ-, wherein n is 1 to 3, more preferably 2.

Most preferably, S_{B} is -PEG₃-PEG₃-.

### Preferred Combinations

It will be understood that the above preferences may be independently combined to give preferred —Z¹ and —Z²—Z¹ moieties.

Some preferred —Z¹ and —Z²—Z¹ moieties are shown below (in each case, - - - indicates the point of attachment to the side chain of the amino acid component of Ψ:

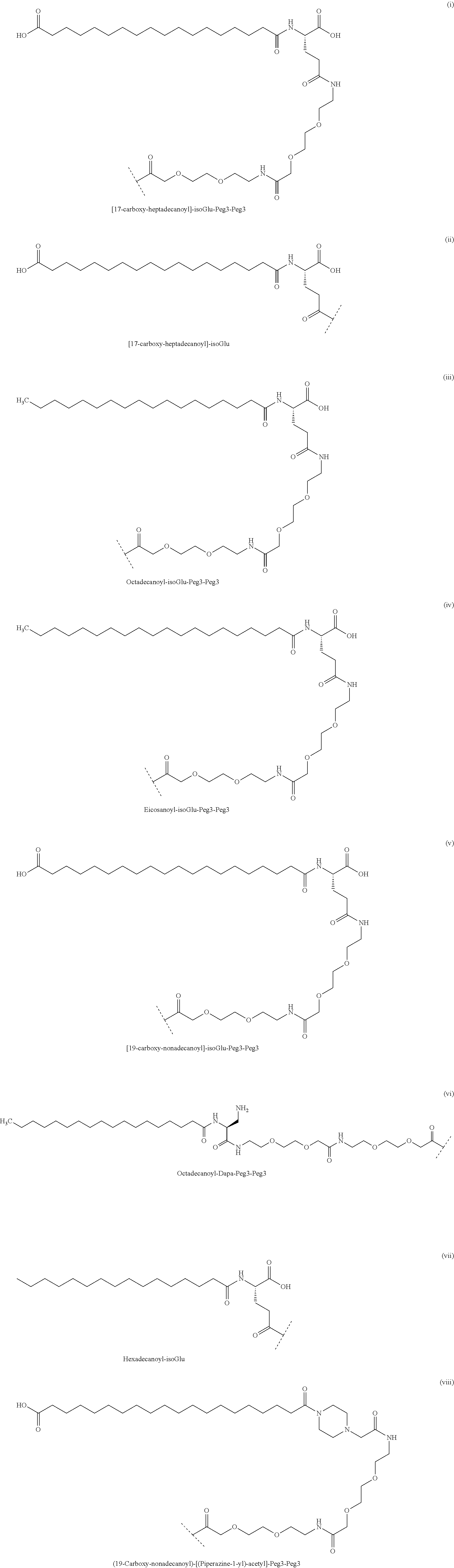

The skilled person will be well aware of suitable techniques for preparing the compounds employed in the context of the invention. For examples of suitable chemistry, see, e.g., WO98/08871, WO00/55184, WO00/55119, Madsen et al. (J. Med. Chem. 2007, 50, 6126-32), and Knudsen et al. 2000 (J. Med Chem. 43, 1664-1669).

### Clinical Utility

The GIP analogue compounds employed in the context of the invention may provide an attractive treatment option for metabolic diseases including obesity, diabetes mellitus (diabetes), obesity-related disorders, and diabetes-related disorders. The GIP analogue compounds of the present invention may be particular effective in improving glycaemic control and reducing body weight when they are administered in combination with a GLP-1 receptor agonist (as part of the same pharmaceutical formulation or as separate formulations). Glucagon-like peptide-1 receptor agonists also known as GLP-1 receptor agonists or incretin mimetics are agonists of the GLP-1 receptor. One of their advantages over older insulin secretagogues, such as sulfonylureas or meglitinides, is that they have a lower risk of causing hypoglycemia.

Examples of GLP-1 agonists include but are not limited to exenatide (BYETTA®/BYDUREON®), liraglutide (VICTOZA®), semaglutide, lixisenatide (LYXUMIA®), albiglutide (TANZENUM®) and Taspoglutide.

Diabetes comprises a group of metabolic diseases characterized by hyperglycemia resulting from defects in insulin secretion, insulin action, or both. Diabetes is classified into type 1 diabetes, type 2 diabetes and gestational diabetes on the basis on pathogenic characteristics. Type 1 diabetes accounts for 5-10% of all diabetes cases and is caused by auto-immune destruction of insulin-secreting pancreatic β-cells. Acute signs of diabetes include excessive urine production, resulting compensatory thirst and increased fluid intake, blurred vision, unexplained weight loss, lethargy, and changes in energy metabolism. However, in type 2 diabetes symptoms are often not severe or may be absent. The chronic hyperglycemia of diabetes is associated with long-term damage, dysfunction, and failure of various organs, notably the eyes, kidneys, nerves, heart and blood vessels.

Type 2 diabetes accounts for 90-95% of diabetes cases and is a result of a complex set of metabolic disorders. However, symptoms are often not severe or may be absent. Type 2 diabetes is the consequence of endogenous insulin production becoming insufficient to maintain plasma glucose levels below diagnostic thresholds.

Gestational diabetes refers to any degree of glucose intolerance identified during pregnancy.

Pre-diabetes includes impaired fasting glucose and impaired glucose tolerance and refers to those states that occur when blood glucose levels are elevated but below the levels that are established for the clinical diagnosis for diabetes.

A large proportion of people with type 2 diabetes and pre-diabetes are at increased risk of morbidity and mortality due to the high prevalence of additional metabolic risk factors, including abdominal obesity (excessive fat tissue around the abdominal internal organs), atherogenic dyslipidemia (blood fat disorders including high triglycerides, low HDL cholesterol and/or high LDL cholesterol, which foster plaque buildup in artery walls), elevated blood pressure (hypertension) a prothrombotic state (e.g. high fibrinogen or plasminogen activator inhibitor-1 in the blood), and/or a proinflammatory state (e.g., elevated C-reactive protein in the blood).

Conversely, obesity confers an increased risk of developing pre-diabetes, type 2 diabetes as well as, e.g., certain types of cancer, obstructive sleep apnea and gall-bladder disease. Dyslipidemia is associated with increased risk of cardiovascular disease. High Density Lipoprotein (HDL) is of clinical importance since an inverse correlation exists between plasma HDL concentrations and risk of atherosclerotic disease. The majority of cholesterol stored in atherosclerotic plaques originates from LDL and hence an elevated concentration of Low Density Lipoproteins (LDL) is closely associated with atherosclerosis. The HDL/LDL ratio is a clinical risk indictor for atherosclerosis and coronary atherosclerosis in particular.

The GIP analogues of the present invention may be used as pharmaceutical agents for preventing weight gain, promoting weight loss, reducing excess body weight or treating obesity (e.g., by control of appetite, feeding, food intake, calorie intake, and/or energy expenditure and lipolysis), including morbid obesity, as well as associated diseases and health conditions including but not limited to obesity linked inflammation, obesity linked gallbladder disease and obesity induced sleep apnea. The GIP analogues employed in the context of the invention may also be used for treatment of insulin resistance, glucose intolerance, pre-diabetes, increased fasting glucose, type 2 diabetes, hypertension, dyslipidemia (or a combination of these metabolic risk factors), atherosclerosis, arteriosclerosis, coronary heart disease, peripheral artery disease and stroke. These are all conditions which may be associated with obesity. However, the effects of the compounds employed in the context of the invention on these conditions may be mediated in whole or in part via an effect on body weight, or may be independent thereof.

The GIP analogues of the present invention may thus be used for the treatment and/or prevention of any of the diseases, disorders, or conditions described herein, including insulin resistance, glucose intolerance, increased fasting glucose, pre-diabetes, type 1 diabetes, type 2 diabetes, gestational diabetes hypertension, dyslipidemia, or a combination thereof. In certain embodiments, the diabetes related disorder is selected from atherosclerosis, arteriosclerosis, coronary heart disease, peripheral artery disease and stroke; or associated with a condition selected from atherogenic dyslipidemia, blood fat disorders, elevated blood pressure, hypertension, a prothrombotic state, and proinflammatory state, or a combination thereof. In certain embodiments, the blood fat disorder is selected from high triglycerides, low HDL cholesterol, high LDL cholesterol, plaque buildup in artery walls, or a combination thereof. In certain embodiments, the prothrombotic state is selected from high fibrinogen levels in the blood and high plasminogen activator inhibitor-1 levels in the blood. In certain embodiments, the proinflammatory state is an elevated C-reactive protein level in the blood. In certain embodiments, the obesity related disorder is selected from obesity linked inflammation, obesity linked gallbladder disease and obesity induced sleep apnea.

The GIP analogues of the present invention may also be used for the treatment and/or prevention of any of the diseases, disorders, or conditions associated with diabetes related osteoporosis including increased risk of bone fractures (Khazai N. B. et al, 2009, Current Opinion in Endocrinology, Diabetes and Obesity, vol. 16, no. 6, 435-445). The increase in fracture risk is likely to be related to impaired bone quality rather than to bone mineral density. The related mechanisms, due at least in part to hyperglycemia, neuropathy, and higher incidence of hypovitaminosis D, are not yet fully understood (Takiishi T et al, 2010, Endocrinology and Metabolism Clinics of North America, vol. 39, no. 2, 419-446).

In some embodiments, the invention also provides a therapeutic kit comprising a GIP analogue (e.g., GIP agonist compound) of the present invention, optionally in combination with a pharmaceutically acceptable carrier. In some embodiments, the invention provides a device comprising a GIP analogue of the invention for delivery of the GIP analogue to a subject.

### Pharmaceutical Compositions

The GIP analogues (e.g., GIP agonist compounds) of the present invention, or salts or solvates thereof, may be formulated as pharmaceutical compositions prepared for storage or administration, which typically comprise a therapeutically effective amount of a compound employed in the context of the invention, or a salt or solvate thereof, in a pharmaceutically acceptable carrier. In some embodiments, the pharmaceutical composition is formulated as a liquid suitable for administration by injection or infusion, or which is formulated to cause slow release of the GIP analogue.

The therapeutically effective amount of a compound of the present invention will depend, e.g., on the route of administration, the type of mammal being treated, and the physical characteristics of the specific mammal under consideration. These factors and their relationship to determining this amount are well known to skilled practitioners in the medical arts. This amount and the method of administration can be tailored to achieve optimal efficacy, and may depend on such factors as weight, diet, concurrent medication and other factors, well known to those skilled in the medical arts. The dosage sizes and dosing regimen most appropriate for human use may be guided by the results obtained by the present invention, and may be confirmed in properly designed clinical trials.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person. The term “pharmaceutically acceptable carrier” includes any of the standard pharmaceutical carriers. Pharmaceutically acceptable carriers for therapeutic use are well known in the pharmaceutical art, and are described, for example, in Remington's Pharmaceutical Sciences, Mack Publishing Co. (A. R. Gennaro edit. 1985). For example, sterile saline and phosphate-buffered saline at slightly acidic or physiological pH may be used. Suitable pH buffering agents may be, e.g., phosphate, citrate, acetate, lactate, maleate, tris/hydroxymethyl)aminomethane (TRIS), N-Tris(hydroxymethyl)methyl-3-aminopropanesulphonic acid (TAPS), ammonium bicarbonate, diethanolamine, histidine, which in certain embodiments is a preferred buffer, arginine, lysine, or acetate or mixtures thereof. The term further encompasses any agents listed in the US Pharmacopeia for use in animals, including humans.

The term “pharmaceutically acceptable salt” refers to a salt of the compound. Salts include pharmaceutically acceptable salts, such as, e.g., acid addition salts and basic salts. Examples of acid addition salts include hydrochloride salts, citrate salts and acetate salts. Examples of basic salts include salts where the cation is selected from alkali metals, such as sodium and potassium, alkaline earth metals such as calcium, and ammonium ions ⁺N(R³)₃(R⁴), where R³ and R⁴ independently designate optionally substituted C₁₋₆-alkyl, optionally substituted C₂₋₆-alkenyl, optionally substituted aryl, or optionally substituted heteroaryl. Other examples of pharmaceutically acceptable salts are described in “Remington's Pharmaceutical Sciences”, 17th edition. Ed. Alfonso R. Gennaro (Ed.), Mark Publishing Company, Easton, PA, U.S.A., 1985 and more recent editions, and in the Encyclopaedia of Pharmaceutical Technology.

“Treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” may also mean prolonging survival as compared to expected survival if not receiving treatment. “Treatment” is an intervention performed with the intention of preventing the development or altering the pathology of a disorder. Accordingly, “treatment” refers to both therapeutic treatment and prophylactic or preventative measures in certain embodiments. Those in need of treatment include those already with the disorder as well as those in which the disorder is to be prevented. By treatment is meant inhibiting or reducing an increase in pathology or symptoms (e.g. weight gain, hyperglycemia) when compared to the absence of treatment, and is not necessarily meant to imply complete cessation of the relevant condition.

The pharmaceutical compositions of the invention may be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. It may be provided in single dose injectable form, for example in the form of an injection pen. Compositions may be formulated for any suitable route and means of administration. Pharmaceutically acceptable carriers or diluents include those used in formulations suitable for oral, rectal, nasal or parenteral (including subcutaneous, intramuscular, intravenous, intradermal, and transdermal) administration. The formulations may conveniently be presented in unit dosage form and may be prepared by any of the methods well known in the art of pharmacy. Subcutaneous or transdermal modes of administration may be particularly suitable for certain of the compounds described herein.

### Combination Therapy

In certain embodiments, a GIP-analogue employed in the context of the invention may be administered as part of a combination therapy with at least one other agent for treatment of diabetes, obesity, dyslipidemia, or hypertension.

In such cases, the at least two active agents may be given together or separately, and as part of the same pharmaceutical formulation or as separate formulations. Thus, the GIP analogue employed in the context of the invention (or the salt or solvate thereof) may be used in combination with an antidiabetic agent including but not limited to a glucagon-like peptide receptor 1 agonist, metformin, a sulfonylurea, a glinide, a DPP-IV inhibitor, a glitazone, or insulin. In certain embodiments, the compound or salt or solvate thereof is used in combination with insulin, DPP-IV inhibitor, sulfonylurea or metformin, particularly sulfonylurea or metformin, for achieving adequate glycemic control. In certain preferred embodiments, the compound or salt or solvate thereof is used in combination with insulin or an insulin analogue for achieving adequate glycemic control. Examples of insulin analogues include but are not limited to LANTUS®, NOVORAPID®, HUMALOG®, NOVOMIX®, ACTRAPHANE HM®, LEVEMIR®, and APIDRAR.

In certain embodiments, the GIP analogue or salt or solvate thereof may further be used in combination with one or more of an anti-obesity agent, including but not limited to a glucagon-like peptide receptor 1 agonist, peptide YY or analogue thereof, cannabinoid receptor 1 antagonist, lipase inhibitor, melanocortin receptor 4 agonist, or melanin concentrating hormone receptor 1 antagonist.

In certain embodiments, the GIP analogue or salt or solvate thereof may be used in combination with an anti-hypertension agent, including but not limited to an angiotensin-converting enzyme inhibitor, angiotensin II receptor blocker, diuretics, beta-blocker, or calcium channel blocker.

In certain embodiments, the GIP analogue or salt thereof may be used in combination with an anti-dyslipidemia agent, including but not limited to a statin, a fibrate, a niacin and/or a cholesterol absorption inhibitor.

### Synthesis of Compounds of the Invention

A nucleic acid molecule may encode the amino acid sequence of any of Formula I to Ill or a precursor thereof. The amino acid sequence encoded can be regarded as a precursor of a compound of the invention.

Typically, such nucleic acid sequences will be provided as expression constructs wherein the encoding nucleic acid is in functional linkage with appropriate control sequences to direct its expression. The expression construct may be provided in the context of a host cell capable of expressing (and optionally also secreting) the amio acid precursor, or in a cell-free expression system.

The invention provides a method of producing a GIP analogue of the invention, the method comprising expressing an amino acid precursor of the GIP analogue and modifying the precursor to provide the GIP analogue. The modification may comprise chemical modification of a Lys, Arg or Cys residue present at position 17 to introduce the lipophilic moiety, modification of the N- or C-terminus, and/or modification of any other amino acid side chains in the molecule (e.g. to introduce a non-naturally occurring amino acid residue).

The compounds of the invention may also be manufactured by standard peptide synthetic methods, e.g. by standard solid-phase or liquid-phase methodology, either stepwise or by fragment assembly, and isolating and purifying the final peptide compound product, or by any combinations of recombinant and synthetic methods.

It may be preferable to synthesize the peptide compounds of the invention by means of solid-phase or liquid-phase peptide synthesis. In this context, reference may be made to WO 98/11125 or, inter alia, Fields, G. B. et al., “Principles and Practice of Solid-Phase Peptide Synthesis”; in: Synthetic Peptides, Gregory A. Grant (ed.), Oxford University Press (2nd edition, 2002) and the synthesis examples herein.

### EXAMPLES

The following examples demonstrate certain embodiments of the present invention. However, it is to be understood that these examples neither purport nor are they intended to be wholly definitive as to conditions and scope of this invention. The examples were carried out using standard techniques, which are well known and routine to those of skill in the art, except where otherwise described in detail. The following examples are presented for illustrative purposes only, and should not be construed in any way as limiting the scope of this invention.

Disclosed are GIP analogues that exhibit signaling selectivity, and methods for screening these compounds. Signaling selectivity may be, for example, preferential pathway activation or preferential pathway inhibition, or both. The analogue, administered alone or in combination with a GLP-1 agonist, may be useful for the treatment and/or prevention of diseases or conditions caused or characterized by excess body weight, including, but not limited to, obesity, morbid obesity, obesity linked inflammation, obesity linked gallbladder disease, obesity induced sleep apnea, metabolic syndrome, pre-diabetes, insulin resistance, glucose intolerance, type 2 diabetes, type I diabetes, hypertension, atherogenic dyslipidemia, atherosclerosis, arteriosclerosis, coronary heart disease, peripheral artery disease, and stroke or microvascular disease.

While some embodiments of the invention have been described by way of illustration, it will be apparent that the invention can be put into practice with many different modifications, variations and adaptations, and with the use of numerous equivalents or alternative solutions that are within the scope of persons skilled in the art, without departing from the spirit of the invention or exceeding the scope of the claims.

All publications, patents, and patent applications referred to herein are herein incorporated by reference in their entirety to the same extent as if each individual publication, patent or patent application was specifically and individually indicated to be incorporated by reference in its entirety.

The methods used in the instant invention are described below, except where expressly indicated otherwise.

### Example 1

### General Synthesis of Acylated GIP Analogues

Solid phase peptide synthesis was performed on a CEM Liberty Peptide Synthesizer using standard Fmoc chemistry. TentaGel S Ram resin (1 g; 0.25 mmol/g) was swelled in NMP (10 ml) prior to use and transferred between tube and reaction vessel using DCM and NMP.

### Coupling

An Fmoc-amino acid in DMF/DCM (2:1; 0.2 M; 5 ml) was added to the resin in a CEM Discover microwave unit together with HATU/DMF or COMU/DMF (0.5 M; 2 ml) and DIPEA-DMF/DCM (2:1) (2.0 M; 1 ml). The coupling mixture was heated to 75° C. for 5 min while nitrogen was bubbled through the mixture. The resin was then washed with DMF (4×10 ml).

### Deprotection

Piperidine/DMF (20%; 10 ml) was added to the resin for initial deprotection and the mixture was heated by microwaves (30 sec; 40° C.). The reaction vessel was drained and a second portion of piperidine/NMP (20%; 10 ml) was added and heated (75° C.; 3 min.) again. The resin was then washed with DMF (6×10 ml).

### Side Chain Acylation

Fmoc-Lys(ivDde)-OH or alternatively another amino acid with an orthogonal side chain protective group was introduced at the position of the acylation. The N-terminal of the peptide backbone was then Boc-protected using Boc2O or alternatively by using a Boc-protected amino acid in the last coupling. While the peptide was still attached to the resin, the orthogonal side chain protective group was selectively cleaved using freshly prepared hydrazine hydrate (2-4%) in NMP for 2×15 min. The unprotected lysine side chain was first coupled with Fmoc-Glu-OtBu or another spacer amino acid, which was deprotected with piperidine and acylated with a lipophilic moiety using the peptide coupling methodology as described above.

Abbreviations employed are as follows:
COMU: 1-[(1-(cyano-2-ethoxy-2-oxoethylideneaminooxy)-dimethylamino-morpholinomethylene)]methanaminium hexaflourophosphate
ivDde: 1-(4,4-dimethyl-2,6-dioxocyclohexylidene)3-methyl-butyl
Dde: 1-(4,4-dimethyl-2,6-dioxocyclohexylidene)-ethyl
DCM: dichloromethane
DMF: N,N-dimethylformamide
DIPEA: diisopropylethylamine
EtOH: ethanol
Et₂O: diethyl ether
HATU: N-[(dimethylamino)-1H-1,2,3-triazol[4,5-b]pyridine-1-ylmethylene]-N-methylmethanaminium hexafluorophosphate N-oxide
MeCN: acetonitrile
NMP: N-methylpyrrolidone
TFA: trifluoroacetic acid
TIS: triisopropylsilane

### Cleavage

The resin was washed with EtOH (3×10 ml) and Et20 (3×10 ml) and dried to constant weight at room temperature (r.t.). The crude peptide was cleaved from the resin by treatment with TFA/TIS/water (95/2.5/2.5; 40 ml, 2 h; r.t.). Most of the TFA was removed at reduced pressure and the crude peptide was precipitated and washed three times with diethylether and dried to constant weight at room temperature.

### HPLC Purification of the Crude Peptide

The crude peptide was purified to greater than 90% by preparative reverse phase HPLC using a PerSeptive Biosystems VISION Workstation equipped with a C-18 column (5 cm; 10 μm) and a fraction collector and run at 35 ml/min with a gradient of buffer A (0.1% TFA, aq.) and buffer B (0.1% TFA, 90% MeCN, aq.). Fractions were analyzed by analytical HPLC and MS and relevant fractions were pooled and lyophilized. The final product was characterized by HPLC and MS.

The synthesized compounds are shown in Table 1.

TABLE 1

| Compound | | SEQ ID |
|---|---|---|
| No. | Sequence | NO. |
| 1 | H-Y-Aib-EGTFISDYSIELDK- | 89 |
| | K(Hexadecanoyl-isoGlu)- | |
| | HQQDFVNWLLAQGPSSGAPPPS-NH₂ | |
| 2 | H-Y-Aib-EGTFISDYSIELD- | 90 |
| | K(Hexadecanoyl-isoGlu)- | |
| | IHQQDFVNWLLAQGPSSGAPPPS-NH₂ | |
| 3 | H-Y-Aib-EGTFISDYSIELEK- | 91 |
| | K(Hexadecanoyl-isoGlu)- | |
| | HQQDFVNWLLAQGPSSGAPPPS-NH₂ | |
| 4 | H-Y-Aib-EGTFISDYSIELDKIHQQDF | 92 |
| | VNWLLAQGPSSGAPPPS-K([19- | |
| | carboxy-nonadecanoyl]- | |
| | isoGlu-Peg3-Peg3)-NH₂ | |
| 5 | H-Y-Aib-EGTFISDYSIELDKIH | 93 |
| | QQDFVNWLLAQGPSSGAPPPS- | |
| | K(Hexadecanoyl-isoGlu)-NH₂ | |
| 6 | H-Y-Aib-EGTFISDYSIELDKIHQ | 94 |
| | QDFVNWLLAQ-K(Hexadecanoyl- | |
| | isoGlu)-NH₂ | |
| 7 | H-Y-Aib-EGTFISDYSIELDKIHQQ | 95 |
| | DFVNWLLAQKG-K(Hexadecanoyl- | |
| | isoGlu)-NH₂ | |
| 8 | H-Y-Aib-EGTFISDYSIELDK- | 96 |
| | K([19-carboxy-nonadecanoyl]- | |
| | isoGlu-Peg3-Peg3)-HQQDFVNYLL | |
| | AQGPSSGAPPPS-NH₂ | |
| 9 | H-Y-Aib-EGTFISDYSIELDK- | 97 |
| | K([19-carboxy-nonadecanoyl]- | |
| | isoGlu-Peg3-Peg3)-HQQDFVNWLL | |
| | AQGPSSGAPPPS-NH₂ | |
| 10 | H-Y-Aib-EGTFISDYSIELDK- | 98 |
| | K([19-carboxy-nonadecanoyl]- | |
| | isoGlu-Peg3-Peg3)-AAQDFVNWLL | |
| | AQGPSSGAPPPS-NH₂ | |
| 11 | H-Y-Aib-EGTFISDYSIELEK- | 99 |
| | K([19-carboxy-nonadecanoyl]- | |
| | isoGlu-Peg3-Peg3)-AAKEFVNWLL | |
| | AQGPSSGAPPPS-NH₂ | |
| 12 | H-Y-Aib-EGTFISDYSIELEK- | 100 |
| | K([19-carboxy-nonadecanoyl]- | |
| | isoGlu-Peg3-Peg3)-AQRAFVEWL | |
| | LAQGPSSGAPPPS-NH₂ | |
| 13 | H-Y-Aib-EGTFISDYSIELEKIAQRAF | 101 |
| | VEWLLAQGPSSGAPPPS- | |
| | K([19-carboxy-nonadecanoyl]- | |
| | isoGlu-Peg3-Peg3)-NH₂ | |
| 14 | H-Y-Aib-EGTFISDYSIELEKIAQRAFV | 102 |
| | EWLLAQ-K([19-carboxy- | |
| | Nonadecanoyl]-isoGlu-Peg3- | |
| | Peg3)-NH₂ | |
| 15 | H-Y-Aib-EGTFISDYSIELDK- | 103 |
| | K([19-carboxy-nonadecanoyl]- | |
| | isoGlu-Peg3-Peg3)-AAQDFVNWLLA | |
| | GPSSGAPPPS-NH₂ | |
| 16 | H-Y-Aib-EGTFISDYSIELDKIAAQD | 104 |
| | FVNWLLAGPSSGAPPPS-K([19- | |
| | carboxy-nonadecanoyl]-isoGlu- | |
| | Peg3-Peg3)-NH₂ | |
| 17 | H-Y-Aib-EGTFISDYSIELDK- | 105 |
| | K([19-Carboxy-nonadecanoyl]- | |
| | isoGlu-Peg3-Peg3)-AQRAFVEWLL | |
| | AQGPSSGAPPPS-NH₂ | |
| 18 | H-Y-Aib-EGTFISDYSIELDK- | 106 |
| | K([19-Carboxy-nonadecanoyl]- | |
| | isoGlu-Peg3-Peg3)-AQRAFIEWLL | |
| | AQGPSSGAPPPS-NH₂ | |
| 19 | H-Y-Aib-EGTFISDYSIELDK- | 107 |
| | K((19-Carboxy-nonadecanoyl)- | |
| | [(Piperazine-1-yl)-acetyl]- | |
| | Peg3-Peg3)-AQRAFIEWLLAQGPSS | |
| | GAPPPS-NH₂ | |
| 20 | H-Y-Aib-EGTFISDYSIELDK- | 108 |
| | K((19-Carboxy-nonadecanoyl)- | |
| | [(Piperazine-1-yl)-acetyl]- | |
| | Peg3-Peg3)-AQRAFVEWLLAQGPSS | |
| | GAPPPS-NH₂ | |
| 21 | H-Y-Aib-EGTFISDYSIELDK- | 109 |
| | K((19-Carboxy-nonadecanoyl)- | |
| | [(Piperazine-1-yl)-acetyl]- | |
| | Peg3-Peg3)-AQKEFVEWLLAAGPSSG | |
| | APPPS-NH₂ | |
| 22 | H-Y-Aib-EGTFISDYSIELDK- | 110 |
| | K([19-Carboxy-nonadecanoyl]- | |
| | isoGlu-Peg3-Peg3)-AQKEFVEWLL | |
| | AAGPSSGAPPPS-NH₂ | |
| 23 | H-Y-Aib-EGTFISDYSIELDKIAQRA | 111 |
| | FIEWLLAGPSSGAPPPS- | |
| | K([19-Carboxy-nonadecanoyl]- | |
| | isoGlu-Peg3-Peg3)-NH₂ | |
| 24 | H-Y-Aib-EGTFISDYSIELDKIAQ | 112 |
| | KEFIEWLLAGPSSGAPPPS- | |
| | K([19-Carboxy-nonadecanoyl]- | |
| | isoGlu-Peg3-Peg3)-NH₂ | |
| 25 | H-Y-Aib-EGTFISDYSIELDKIAAQD | 113 |
| | FIEWLLAGPSSGAPPPS- | |
| | K([19-Carboxy-nonadecanoyl]- | |
| | isoGlu-Peg3-Peg3)-NH₂ | |
| 26 | H-Y-Aib-EGTFISDYSIELDKIAAQDF | 114 |
| | IEWLLAGPSSGAPPPS- | |
| | K((19-Carboxy-nonadecanoyl)- | |
| | [(Piperazine-1-yl)-acetyl]- | |
| | Peg3-Peg3)-NH₂ | |
| 27 | H-Y-Aib-EGTFISDYSIELDKIAAQD | 115 |
| | FVEWLLAGPSSGAPPPS- | |
| | K([19-Carboxy-nonadecanoyl]- | |
| | isoGlu-Peg3-Peg3)-NH₂ | |
| 28 | H-Y-Aib-EGTFISDYSIELDKIAQRAF | 116 |
| | IEWLLAQGPSSGAPPPS- | |
| | K([19-Carboxy-nonadecanoyl]- | |
| | isoGlu-Peg3-Peg3)-NH₂ | |
| 29 | H-Y-Aib-EGTFISDYSIELDK- | 117 |
| | K([19-Carboxy-nonadecanoyl]- | |
| | isoGlu-Peg3-Peg3)-AAQAFVNWLL | |
| | AGPSSGAPPPS-NH₂ | |
| 30 | H-Y-Aib-EGTFISDYSIELDK- | 118 |
| | K([19-Carboxy-nonadecanoyl]- | |
| | isoGlu-Peg3-Peg3)-AAQDFVNWLL | |
| | AAGPSSGAPPPS-NH₂ | |
| 31 | H-Y-Aib-EGTFISDYSIELDK- | 119 |
| | K([19-Carboxy-nonadecanoyl]- | |
| | isoGlu-Peg3-Peg3)-AAQDFINWLL | |
| | AGPSSGAPPPS-NH₂ | |
| 32 | H-Y-Aib-EGTFISDYSIELDK- | 120 |
| | K([19-Carboxy-nonadecanoyl]- | |
| | isoGlu-Peg3-Peg3)-AAQDFIEWLL | |
| | AGPSSGAPPPS-NH₂ | |
| 33 | H-Y-Aib-EGTFISDYSIELDK- | 121 |
| | K((19-Carboxy-nonadecanoyl)- | |
| | [(Piperazine-1-yl)-acetyl]- | |
| | Peg3-Peg3)-AAQDFIEWLLAGPSS | |
| | GAPPPS-NH₂ | |
| 34 | H-Y-Aib-EGTFISDYSIELD- | 122 |
| | K([19-Carboxy-nonadecanoyl]- | |
| | isoGlu-Peg3-Peg3)-IAQRAFIEWL | |
| | LAQGPSSGAPPPS-NH₂ | |
| 35 | H-Y-Aib-EGTFISDYS- | 123 |
| | K([19-Carboxy-nonadecanoyl]- | |
| | isoGlu-Peg3-Peg3)-ELDKIAQRAF | |
| | IEWLLAQGPSSGAPPPS-NH₂ | |
| 36 | H-Y-DAla-EGTFISDYSIELDK- | 124 |
| | K([19-Carboxy-nonadecanoyl]- | |
| | isoGlu-Peg3-Peg3)-AQRAFIEWLL | |
| | AQGPSSGAPPPS-NH₂ | |
| 37 | H-Y-DAla-EGTFISDYSIELDKIAAQD | 125 |
| | FIEWLLAGPSSGAPPPS- | |
| | K((19-Carboxy-nonadecanoyl)- | |
| | [(Piperazine-1-yl)-acetyl]- | |
| | Peg3-Peg3)-NH₂ | |
| 38 | H-Y-Aib-EGTFISDYSIELDK- | 126 |
| | K([19-Carboxy-nonadecanoyl]- | |
| | isoGlu-Peg3-Peg3)-AAQDFIEWL | |
| | LAQGPSSGAPPPS-NH₂ | |
| 39 | H-Y-Aib-EGTFISDYSIELDK- | 127 |
| | K([19-Carboxy-nonadecanoyl]- | |
| | isoGlu-Peg3-Peg3)-AAQDFINWL | |
| | LAQGPSSGAPPPS-NH₂ | |
| 40 | H-Y-Aib-EGTFISDYSIELDK- | 128 |
| | K([19-Carboxy-nonadecanoyl]- | |
| | isoGlu-Peg3-Peg3)-AAQAFIEWL | |
| | LAQGPSSGAPPPS-NH₂ | |
| 41 | H-Y-Aib-EGTFISDYSIELDK- | 129 |
| | K((19-Carboxy-nonadecanoyl)- | |
| | [(Piperazine-1-yl)-acetyl]- | |
| | Peg3-Peg3)-AAQAFIEWLLAQGPS | |
| | SGAPPPS-NH₂ | |

### Synthesis of Compound No. 9

Solid phase peptide synthesis was performed on a CEM Liberty Peptide Synthesizer using standard Fmoc chemistry. TentaGel S Ram S resin (1.05 g; 0.23 mmol/g) was swelled in DMF (10 ml) prior to use and transferred between tube and reaction vessel using DCM and DMF.

### Coupling

An Fmoc-amino acid in DMF/DCM (2:1 ; 0.2 M; 5 ml) was added to the resin in a CEM Discover microwave unit together with COMU/DMF (0.5 M; 2 ml) and DIPEA-DMF/DCM (2:1) (2.0 M; 1 ml). The coupling mixture was heated to 75° C. for 5 min while nitrogen was bubbled through the mixture. The resin was then washed with DMF (4×10 ml). Fmoc-Tyr(OtBu)-Ser(Psi Me,Me)-OH pseudoproline was used for amino acid number 29 and 30 counting from the C-terminal. Lys17 was incorporated as Fmoc-Lys(Dde)-OH for orthogonal coupling. The first 9 amino acids and amino acid number 24 (counting from the C-terminal) was double couple meaning the building block was coupled twice before deprotection. Boc-Tyr(tBu)-OH was incorporated as the final building block in the N-terminal.

### Deprotection

Piperidine/DMF (20%; 10 ml) was added to the resin for initial deprotection and the mixture was heated by microwaves (30 sec; 40° C.). The reaction vessel was drained and a second portion of piperidine/DMF (20%; 10 ml) was added and heated (75° C.; 3 min.) again. The resin was then washed with DMF (6×10 ml).

Side chain acylation

While the peptide was still attached to the resin, the orthogonal side chain protective group was selectively cleaved using freshly prepared hydrazine hydrate (2-4%) in NMP for 2×15 min. The unprotected lysine side chain was first coupled with Fmoc-Glu-OtBu and the two Peg3 buildingblocks using standard coupling and deprotection conditions as explained above. Lastly the lipophilic moiety was incorporated as a 19-carboxy-nonadecanoic acid mono tert butyl ester again using standard coupling conditions.

### Cleavage

The resin was washed with EtOH (3×10 ml) and Et2O (3×10 ml) and dried to constant weight at room temperature (r.t.). The crude peptide was cleaved from the resin by treatment with TFA/TIS/H₂O (95/2.5/2.5; 60 ml, 2 h; r.t.). Most of the TFA was removed at reduced pressure and the crude peptide was precipitated and washed three times with diethylether and dried to constant weight at room temperature.

### HPLC Purification of the Crude Peptide

The crude peptide was first purified from 30% by preparative reverse phase HPLC using a Gilson 331 pump with a Gilson GX281 fraction collector equipped with a Gemini NX 5μ C-18 110A, 10×250 mm column and run at 47 ml/min with a gradient of buffer A (0.1% TFA, aq.) and buffer B (0.1% TFA, 90% MeCN, aq.). Fractions were analyzed by analytical HPLC and MS and relevant fractions were pooled and lyophilized. A second purification was performed using the same method to obtain the product in 96% purify (53 mg) as characterized by HPLC and MS. Calculated monoisotopic mass=5025,54 found 5025,72.

### Example 2

### Human GIP Receptor (GIP R) Activity Assay

In vitro effects of peptide conjugates of the invention were assessed by measuring the induction of cAMP following stimulation of the respective receptor by GIP or analogues of these, as outlined in the invention, using the AlphaSceen® cAMP kit from Perkin-Elmer according to instructions. Briefly, HEK293 cells expressing the human GIP R (stable cell lines generated through transfection of the cDNA for human GIP R and selection of stable clones) were seeded at 30,000 cells/well in 96-well microtiter plates coated with 0.01% poly-L-lysine, and grown for 1 day in culture in 200 μl growth medium (DMEM, 10% FCS, Penicillin (100 IU/ml), Streptomycin (100 μg/ml)). On the day of analysis, growth medium was removed and the cells were washed once with 150 μl Tyrode's buffer (Tyrode's Salts (9.6 g/l), 10 mM HEPES, pH 7.4). Cells were then incubated in 100 μl Assay buffer (0.1% W/V Alkali-treated Casein and 100 μM IBMX in Tyrode's Buffer) containing increasing concentrations of control and test compounds for 15 min at 37° C. The Assay buffer was removed and cells are lysed in 80 μl Lysis buffer (0.1% w/v BSA, 5 mM HEPES, 0.3% v/v Tween-20) per well. From each well 10 μl lysed cells was transferred to a 384-well plate and mixed with 15 μl bead-mix (1 Unit/15 μl anti-cAMP Acceptor Beads, 1 Unit/15 μl Donor Beads, and 1 Unit/15 μl Biotinylated cAMP in Assay Buffer). The plates were mixed and incubated in the dark for an hour at room temperature before measuring using an Envision™ plate reader (Perkin-Elmer).

Results were converted into cAMP concentrations using a cAMP standard curve prepared in KRBH buffer containing 0.1% (v/v) DMSO. The resulting cAMP curves were plotted as absolute cAMP concentrations (nM) over log (test compound concentration) and analyzed using the curve fitting program XLfit.

Parameter calculated to describe both the potency as well as the agonistic activity of each test compound on the receptors were:

EC50, a concentration resulting in a half-maximal elevation of cAMP levels, reflecting the potency of the test compound. The results are summarized in Table 2.

TABLE 2
EC₅₀ average values of the compounds on
the GIP-R compared to control peptide.

| | hGIP-R level |
|---|---|
| Compound | of cAMP (nM) |
| hGIP | 0.003 |
| 1 | 0.008 |
| 2 | 0.013 |
| 3 | 0.014 |
| 4 | 0.013 |
| 5 | 0.014 |
| 6 | 0.032 |
| 7 | 0.018 |
| 8 | 0.009 |
| 9 | 0.008 |
| 10 | 0.007 |
| 11 | 0.009 |
| 12 | 0.009 |
| 13 | 0.014 |
| 14 | 0.024 |
| 15 | 0.012 |
| 16 | 0.016 |
| 17 | 0.007 |
| 18 | 0.006 |
| 19 | 0.006 |
| 20 | 0.007 |
| 21 | 0.007 |
| 22 | 0.005 |
| 23 | 0.010 |
| 24 | 0.008 |
| 25 | 0.032 |
| 26 | 0.017 |
| 27 | 0.013 |
| 28 | 0.007 |
| 29 | 0.014 |
| 30 | 0.009 |
| 31 | 0.012 |
| 32 | 0.020 |
| 33 | 0.014 |
| 34 | 0.011 |
| 35 | 0.008 |
| 36 | 0.006 |
| 37 | 0.016 |
| 38 | 0.001 |
| 39 | 0.007 |
| 40 | 0.010 |
| 41 | 0.014 |

### Example 3

### Activity Assays at Human GIP Receptor (GIP R) and Human GLP-1 Receptor (GLP-1R)

In vitro effects of peptide conjugates were assessed by measuring the induction of cAMP following stimulation of the respective receptor using the AlphaScreen® cAMP kit from Perkin-Elmer according to instructions. Briefly, HEK293 cells expressing the GIP R or the GLP-1R (stable cell lines generated through transfection of expression vector containing the cDNA for the receptor in question and selection of stable clones) were seeded at 30,000 cells/well in 96-well microtiter plates coated with 0.01% poly-L-lysine, and grown for 1 day in culture in 200 μl growth medium (DMEM, 10% FCS, Penicillin (100 IU/ml), Streptomycin (100 μg/ml)). On the day of analysis, growth medium was removed and the cells were washed once with 150 μl Tyrode's buffer (Tyrode's Salts (9.6 g/l), 10 mM HEPES, pH 7.4). Cells were then incubated in 100 μl Assay buffer (0.05% W/V Alkali-treated Casein and 100 μM IBMX in Tyrode's Buffer) containing increasing concentrations of control and test compounds for 15 min at 37° C. The Assay buffer was removed and cells are lysed in 80 μl Lysis buffer (0.1% w/v BSA, 5 mM HEPES, 0.3% v/v Tween-20) per well. From each well 10 μl lysed cells was transferred to a 384-well plate and mixed with 15 μl bead-mix (1 Unit/15 μl anti-cAMP Acceptor Beads, 1 Unit/15 μl Donor Beads, and 1 Unit/15 μl Biotinylated cAMP in Assay Buffer). The plates were mixed and incubated in the dark for an hour at room temperature before measuring using an Envision™ plate reader (Perkin-Elmer).

The cAMP response was normalized relative to a positive and negative control (reference agonist (0.1 nM human GIP or 1 nM Exendin-4) and assay buffer, respectively) to calculate the EC50 and maximal response from the concentration response curve using 4 parameter logistic (4 PL) nonlinear regression model for curve fitting.

The EC50s, a concentration resulting in a half-maximal elevation of cAMP levels, reflecting the potencies of the test agonist compounds are summarized in Table 2a.

TABLE 2a
EC₅₀ average values of the compounds
compared to control peptides.

| | EC50 | EC50 |
|---|---|---|
| Compound | hGIP R (nM) | hGLP1 R (nM) |
| Exendin-4 | NT | 0.004 |
| hGIP | NT | >100 |
| 1 | 0.003 | NA |
| 2 | 0.008 | NT |
| 3 | 0.014 | NA |
| 4 | 0.014 | NT |
| 5 | 0.014 | NT |
| 6 | 0.014 | NT |
| 7 | 0.032 | NA |
| 8 | 0.019 | >10 |
| 9 | 0,009 | >3 |
| 10 | 0.008 | >10 |
| 11 | 0.008 | >3 |
| 12 | 0.009 | >3 |
| 13 | 0.008 | >10 |
| 14 | 0.014 | >10 |
| 15 | 0.024 | >100 |
| 16 | 0.012 | >10 |
| 17 | 0.016 | >3 |
| 18 | 0.007 | >3 |
| 19 | 0.005 | >3 |
| 20 | 0.006 | >3 |
| 21 | 0.006 | >3 |
| 22 | 0.007 | >10 |
| 23 | 0.005 | >10 |
| 24 | 0.010 | >10 |
| 25 | 0.008 | >100 |
| 26 | 0.032 | >10 |
| 27 | 0.017 | >10 |
| 28 | 0.013 | >3 |
| 29 | 0.007 | >100 |
| 30 | 0.014 | >10 |
| 31 | 0.009 | >100 |
| 32 | 0.012 | >100 |
| 33 | 0.020 | >10 |
| 34 | 0.017 | >10 |
| 35 | 0.011 | >3 |
| 36 | 0.006 | >10 |
| 37 | 0.006 | >10 |
| 38 | 0.017 | >100 |
| 39 | 0.001 | >100 |
| 40 | 0.007 | >10 |
| 41 | 0.010 | >10 |

NT = Not tested,
NA = No activity

### Example 4

### Pharmacokinetics of Selected Compounds in Mice

### Method

C57BL/6J mice (males with a body weight of approximately 25 g) were given either a single subcutaneous (s.c.) bolus or a single intravenous (i.v.) bolus of each peptide to be tested.

Following s.c. administration of the selected compounds (50, 100 or 200 nmol/kg), blood samples were drawn at 8 (eight) timepoints up to 96 hours post-dose. Following i.v. administration of the selected compounds (50, 100 or 200 nmol/kg), blood samples were drawn at 8 (eight) timepoints up to 72 hours post-dose. Blood samples were drawn by sublingual bleeding. The dosing vehicle was a phosphate buffer containing mannitol (pH 7.5).

At each sampling time point, samples from two mice were drawn, i.e. 16 mice were included for each compound and each administration route. The mice were euthanized immediately after blood sampling by cervical dislocation. Plasma samples were analyzed after solid phase extraction (SPE) or protein precipitation followed by liquid chromatography mass spectrometry (LC-MS/MS). Mean plasma concentrations were used for calculation of the pharmacokinetic parameters using the non-compartmental approach in Phoenix WinNonlin 6.3. Plasma terminal elimination half-life (T½) was determined as In(2)/λz where λz is the magnitude of the slope of the log linear regression of the log concentration versus time profile during the terminal phase. Bioavailability was determined as AUC_{inf} (s.c.)/AUC_{inf} (i.v.)×100, where AUC_{inf} is the area under the plasma concentration−time curve extrapolated to infinity (AUC_{inf}=AUCₗₐₛₜ+Clast/λz, where Cₗₐₛₜ is the last observed plasma concentration). Tₘₐₓ is the post-dose time where the maximal plasma concentration was observed. The results are summarized in Table 3.

TABLE 3
Terminal elimination half-life (h) and bioavailability in mice
following s.c. and i.v. administration of selected compounds.

| | T½ (h.) | | Tmax (h.) | Bioavailability |
|---|---|---|---|---|
| Compound | i.v. | s.c. | s.c. | s.c. |
| hGIP | 0.1 | — | — | — |
| 10 | 16.9 | 21.1 | 4 | 100%*\hat{ } |
| 12 | 14.7 | 16.8 | 8 | 77%\hat{ } |
| 15 | 19.2 | 16.7 | 8 | 87% |
| 16 | 23.3 | 23.6 | 8 | 81% |
| 13 | 14.4 | 13.7 | 8 | 75% |
| 16 | 19.2 | 16.7 | 8 | 88% |
| 17 | 16.3 | 19.9 | 8 | 56% |
| 18 | 17.6 | 15.1 | 4 | 78% |
| 21 | 24.8 | 21.0 | 8 | 67% |
| 33 | 21.7 | 18.7 | 8 | 78% |
| 35 | 14.5 | 14.5 | 4 | 73% |
| 41 | 17.6 | 16.5 | 8 | 70% |

*The bioavailability was capped to 100%
\hat{ }In a repeated test the bioavalability of Compound 10 was 77% and the bioavailability of Compound 12 was 98%.

### Example 5

### OGTT (Oral Glucose Tolerance Test) in Normal Mice

Male C57BL/6J mice (Charles River, Germany) were maintained on normal chow (Altromin 1324, Brogaarden A/S, Gentofte, Denmark) and domestic quality water with added citric acid to PH˜3.6.

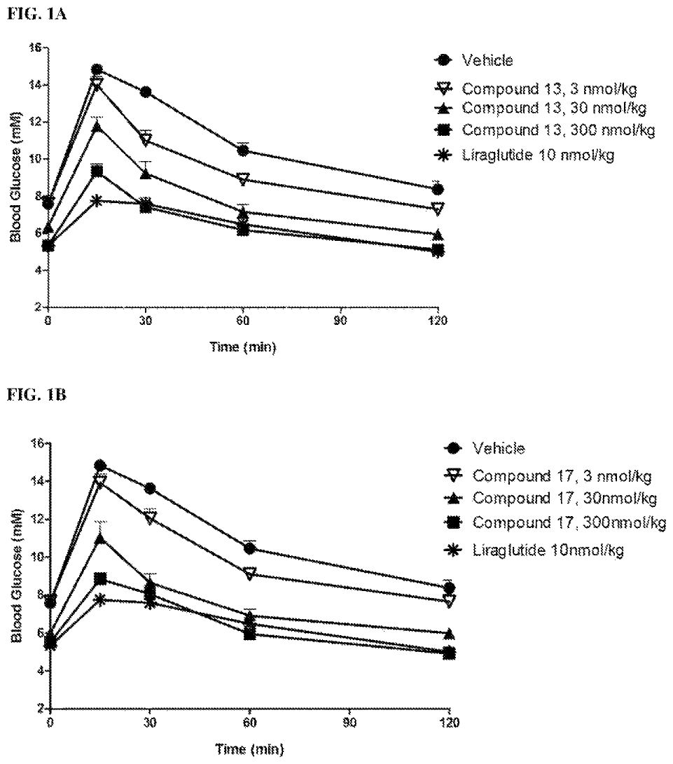

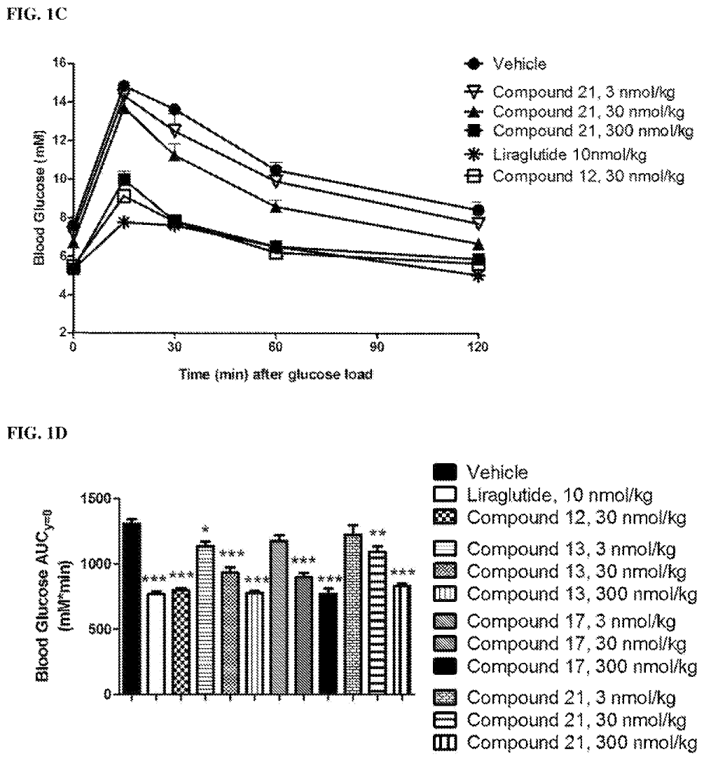

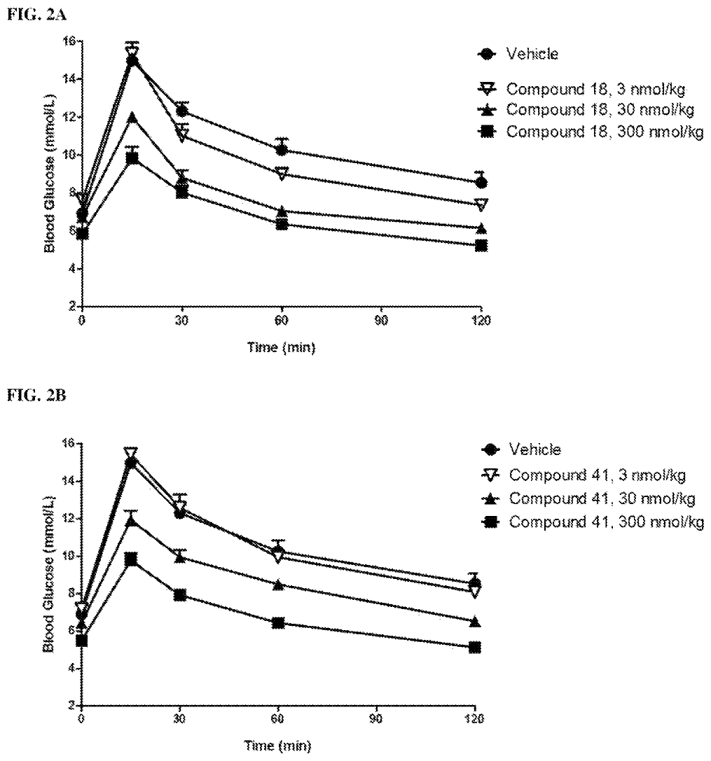

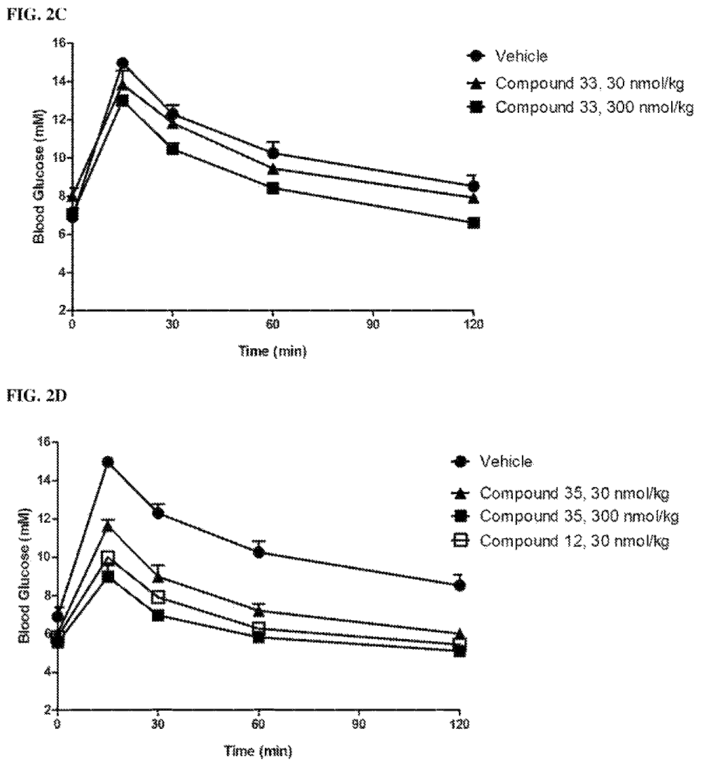

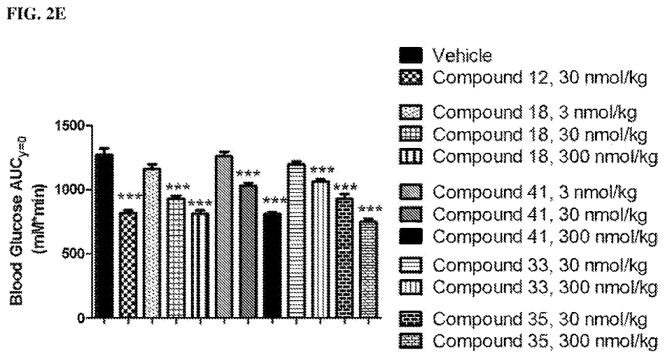

The animals were housed in groups of n=3 in a light-, temperature-, and humidity-controlled room (12:12 h light-dark cycle, with lights on at 06.00-18.00 hr; 21±1° C.; 50-80% relative humidity). Mice, 10-12 weeks old, were fasted 5 hours before the OGTT. GIP receptor agonists (3-300 nmol/kg), the GLP-1 analogue liraglutide (10 nmol/kg) and vehicle were administered (5 ml/kg) subcutaneously (s.c.) 4 hours before the oral gavage of glucose (t=0 min; 2 g/kg; 5 mL/kg). Tail vein blood was sampled at time t=0 (before glucose administration), 15, 30, 60, and 120 min for measurements of blood glucose. Results (blood glucose levels and area under the blood glucose curves (AUC). Data are means±SEM; n=6) from 2 experiments are shown in FIGS. 1 (A-D) and 2 (A-E).

Statistical analyses were performed using Graph Pad Prism version 5. The blood glucose AUCs were compared using one-way ANOVA followed by Dunnett's Multiple Comparison Tests vs. vehicle group. Differences were considered statistically significant at p<0.05. Statistical differences vs vehicle: *p<0.05, **p<0.01, ***p<0.001.

### Example 6

### Sub-Chronic Effects of Co-Treatment of GIP Receptor Agonist and GLP-1 Receptor Agonist on Body Weight in Diet-Induced Obese (DIO) C57BL/6J Mice

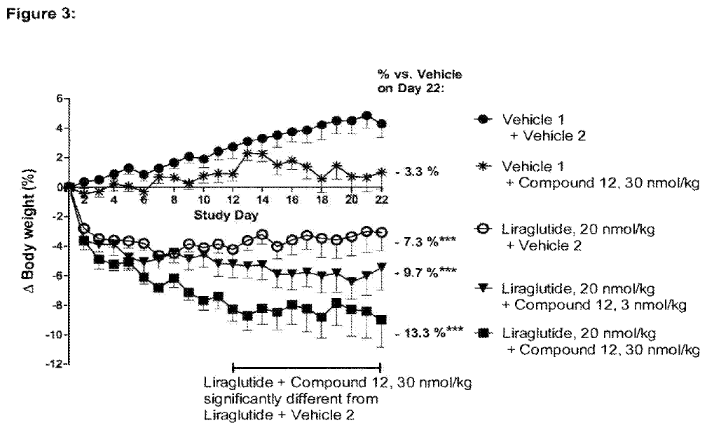

Male C57BL/6J (JAX) mice (Charles River, UK) fed high-fat diet (45% of total energy from fat, D12451 Research Diet Inc.) for approximately 4 months were used. The animals were housed in groups of n=3 in a light-, temperature-, and humidity-controlled room (12:12 h light-dark cycle, with lights on at 07.00-19.00 hr; 21±2° C.; 55±20% relative humidity). Mice were single-housed two weeks prior to start of the mock phase. All mice were mock-treated (once daily s.c. injection of vehicle) for a week to acclimatize the animals to handling and injections. Subsequently, the mice were stratified according to body weight into treatment groups (n=8-9). The average starting body weight was 39-40 grams. Animals were thereafter treated once daily with two separate s.c. injections (3 mL/kg of each injection) from day 1 to day 22. The first injection was with vehicle 1 (25 mM phosphate, 125 mM sodium chloride buffer, pH 7.4) or GLP-1 analogue liraglutide (20 nmol/kg). The second injection was with vehicle 2 (25 mM phosphate, 205 mM D-Mannitol, pH 7.5) or GIP agonist (3 and 30 nmol/kg). The GIP agonist was only dosed every third day of the study (starting on day 1). On other days, GIP agonist was replaced with vehicle 2. The daily injections were given in the morning (at 9.00-10.00). Body weight was determined daily throughout the study. Changes in body weight during the study are shown in FIG. 3 (delta Δ=body weight at each study day−body weight at day 1. Data are means±SEM).

Statistical analyses were performed using Graph Pad Prism version 5. The change in body weight of liraglutide-treated mice was compared with mice co-administered liraglutide and GIP agonist by two-way ANOVA followed by Bonferroni posttests. P<0.05 was considered statistically significant. The change in body weight of vehicle-treated control mice was compared with compound-treated mice by two-way ANOVA followed by Bonferroni posttests; ***p<0.001 vs. vehicle. Statistical differences vs vehicle are shown for day 22 in FIG. 3.

### Example 7

### Sub-Chronic Effects of Co-Treatment of GIP Receptor Agonists and GLP-1 Receptor Agonist on Body Weight in Diet-Induced Obese (DIO) C57BL/6J Mice

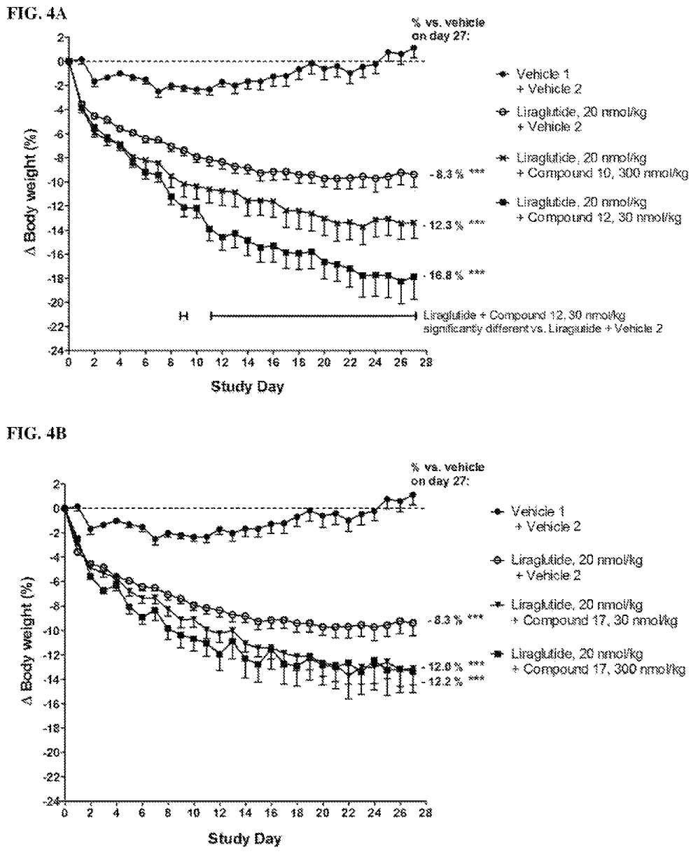

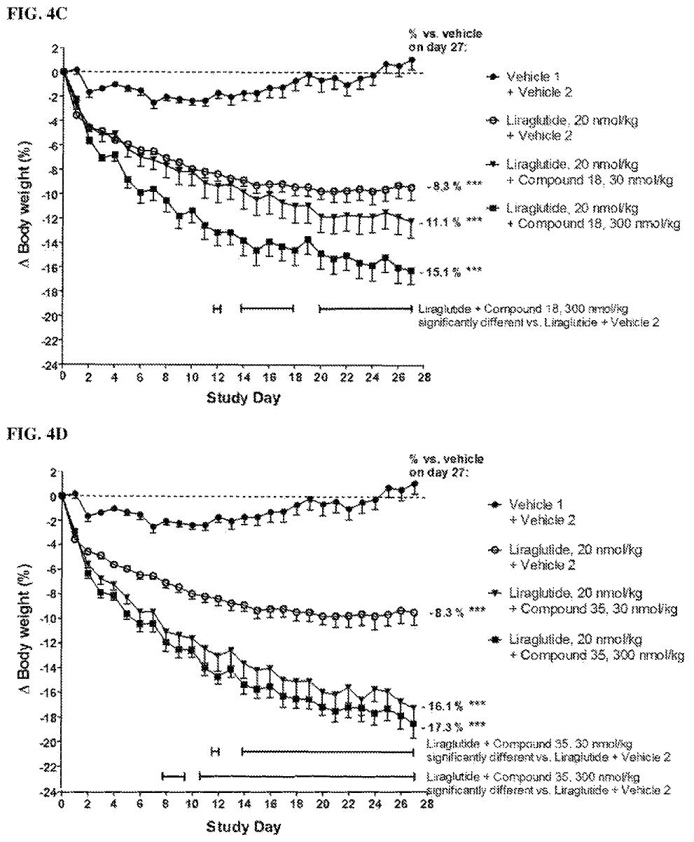

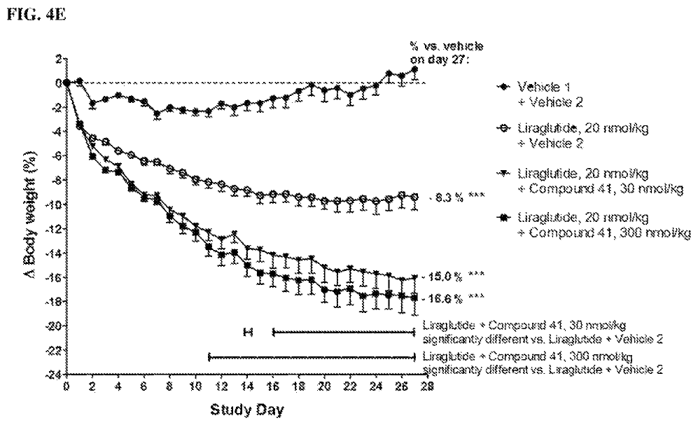

Male C57BL/6J mice (Charles River, Germany) fed high-fat diet (60% of total energy from fat, DIO Rodent Purified 58Y1-58126 from TestDiet) for approximately 5 months were used. The animals were housed in groups of n=3 in a light-, temperature-, and humidity-controlled room (12:12 h light-dark cycle, with lights on at 06.00-18.00 hr; 21±1° C.; 65±15% relative humidity). All mice were mock-treated (once daily s.c. injection of vehicle) for a week to acclimatize the animals to handling and injections. Subsequently, the mice were stratified according to body weight into treatment groups (n=9). The average starting body weight was 40-41 grams. Animals were thereafter treated once daily with two separate s.c. injections (5 mL/kg of each injection) from day 0 to day 27. The first injection was with vehicle 1 (25 mM phosphate, 125 mM sodium chloride buffer, pH 7.4) or GLP-1 analogue liraglutide (20 nmol/kg). The second injection was with vehicle 2 (25 mM phosphate, 205 mM D-Mannitol, pH 7.5) or GIP agonist (30 and/or 300 nmol/kg). The GIP agonist was only dosed every third day of the study (starting on day 0). On other days, GIP agonist was replaced with vehicle 2. The daily injections were given in the morning (at 9.00-10.00). Body weight was determined daily throughout the study. Changes in body weight during the study (delta Δ body weight=body weight at each study day−body weight at day 0. Data are means±SEM) are shown in FIG. 4A (Compound 10 and 12), B (Compound 17), C (Compound 18), D (compound 35) and E (Compound 41).

Statistical analyses were performed for using Graph Pad Prism version 5. The change in body weight of liraglutide-treated mice was compared with mice co-administered liraglutide and GIP agonist by two-way ANOVA followed by Bonferroni posttests. P<0.05 was considered statistically significant (illustrated with lines below the body weight curves). The change in body weight of vehicle-treated control mice was compared with compound-treated mice by two-way ANOVA followed by Bonferroni posttests; ***p<0.001 vs. vehicle. Statistical differences vs vehicle are shown for day 27 (FIG. 4A-E).

1. A GIP analogue represented by the general Formula I:
(SEQ ID NOs: 42 & 86)
R¹-Tyr-X2-Glu-Gly-Thr-Phe-Ile-Ser-Asp-X10-
X11-X12-Glu-Leu-X15-X16-X17-X18-X19-X20-X21-
Phe-X23-X24-X25-Leu-X27-X28-X29-Y1-Y2-R²
(I)
wherein
R¹ is H—, Ac or pGlu (pyroglutamic acid, (S)-(−)-2-pyrrolidone-5-carboxylic acid);
X2 is Aib, Ala, D-Ala, Gly, Ser, N-Me-Ser, Ac3c, Ac4c or Ac5c;
X10 is Tyr, Leu or Ser;
X11 is Ser or Leu;
X12 is Lys, Ψ or Ile;
X15 is Asp or Glu;
X16 is Ser, Glu, Lys or Ψ;
X17 is Ile, Lys, Gln, Arg or Ψ;
X18 is His, Arg or Ala;
X19 is Gln, Lys, Ala or Glu;
X20 is Gln, Lys, Ala, His or Arg;
X21 is Ala, Leu, Asp or Glu;
X23 is Val or Ile;
X24 is Asn or Glu;
X25 is Tyr or Trp;
X27 is Leu, Glu, Ser, Lys or Val;
X28 is Ala, Ser or Arg;
X29 is Aib, Gly, Ala, Gln, Thr, Ser or Lys or is absent;
Y1 is Lys-Gly, Gly-Pro-Ser-Ser-Gly-Ala-Pro-Pro-Pro-Ser (SEQ ID NO: 43), Gly-Pro-Ser-Ser-Gly-Ala-Pro-Pro-Ser (SEQ ID NO: 44), Pro-Ser-Ser-Gly-Ala-Pro-Pro-Pro-Ser (SEQ ID NO: 45), Pro-Ser-Ser-Gly-Ala-Pro-Pro-Ser (SEQ ID NO: 46), Gly-Lys-Lys-Asn-Asp-Trp-Lys-His-Asn-Ile-Thr-Gln (SEQ ID NO: 47) or absent;
Y2 is Ψ or is absent;
R2 is —NH2 or —OH;
wherein Ψ is a residue independently selected from Lys, Arg, Orn and Cys and wherein the side chain of said residue is conjugated to a lipophilic substituent;
and wherein the GIP analogue contains one and only one residue Ψ;
or a pharmaceutically acceptable salt or solvate thereof.

2. (canceled)

3. A GIP analogue represented by the general Formula II:
(SEQ ID NOs: 48 & 87)
R¹-Tyr-X2-Glu-Gly-Thr-Phe-Ile-Ser-Asp-Tyr-
Ser-X12-Glu-Leu-X15-X16-X17-X18-X19-X20-X21-
Phe-X23-X24-X25-Leu-X27-X28-X29-Y1-Y2-R²
(II)
wherein
R¹ is H—, Ac or pGlu;
X2 is Aib, Ala, D-Ala, Gly;
X12 is Lys, Ψ or Ile;
X15 is Asp or Glu;
X16 is Ser, Glu, Lys or Ψ;
X17 is Ile, Lys, Gin, Arg or Ψ;
X18 is His, Arg or Ala;
X19 is Gln or Ala;
X20 is Gln, Lys, Ala, His or Arg;
X21 is Ala, Asp or Glu;
X23 is Ile or Val;
X24 is Asn or Glu;
X25 is Tyr or Trp;
X27 is Leu, Glu, Ser, Lys or Val;
X28 is Ala, Ser or Arg;
X29 is Aib, Gly, Ala, Gln, Thr, Ser or Lys or is absent;
Y1 is Lys-Gly, Gly-Pro-Ser-Ser-Gly-Ala-Pro-Pro-Pro-Ser (SEQ ID NO: 43), Gly-Pro-Ser-Ser-Gly-Ala-Pro-Pro-Ser (SEQ ID NO: 44), Pro-Ser-Ser-Gly-Ala-Pro-Pro-Pro-Ser (SEQ ID NO: 45), Pro-Ser-Ser-Gly-Ala-Pro-Pro-Ser (SEQ ID NO: 46), Gly-Lys-Lys-Asn-Asp-Trp-Lys-His-Asn-Ile-Thr-Gin (SEQ ID NO: 47) or absent;
Y2 is Ψ or is absent;
R² is —NH₂ or —OH;
wherein Ψ is a Lys residue wherein the side chain of said Lys residue is conjugated to a lipophilic substituent;
and wherein the GIP analogue contains one and only one residue Ψ;
or a pharmaceutically acceptable salt or solvate thereof.

4. (canceled)

5. A GIP analogue represented by the general Formula III:
R¹-Tyr-Aib-Glu-Gly-Thr-Phe-Ile-Ser-Asp-
Tyr-Ser-Ile-Glu-Leu-X15-X16-X17-X18-X19-
X20-X21-Phe-Val-X24-X25-Leu-Leu-Ala-X29-
YL-Y2-R²
(III)
wherein
R¹ is H—, Ac or pGlu;
X15 is Asp or Glu;
X16 is Lys or Ψ;
X17 is Ile or Ψ;
X18 is His or Ala
X19 is Gln or Ala;
X20 is Gln, Lys or Arg;
X21 is Ala, Asp or Glu;
X24 is Asn or Glu;
X25 is Tyr or Trp
X28 is Ala, Ser or Arg;
X29 is Gln or is absent;
Y1 is Lys-Gly, Gly-Pro-Ser-Ser-Gly-Ala-Pro-Pro-Pro-Ser, Gly-Pro-Ser-Ser-Gly-Ala-Pro-Pro-Ser, Pro-Ser-Ser-Gly-Ala-Pro-Pro-Pro-Ser, Pro-Ser-Ser-Gly-Ala-Pro-Pro-Ser, Gly-Lys-Lys-Asn-Asp-Trp-Lys-His-Asn-Ile-Thr-Gln or absent;
Y2 is Ψ or is absent;
R² is —NH₂ or —OH;
wherein Ψ is a residue independently selected from Lys, Arg, Orn and Cys and wherein the side chain of said residue is conjugated to a lipophilic substituent;
and wherein the GIP analogue contains one and only one residue Ψ;
or a pharmaceutically acceptable salt or solvate thereof.

6. (canceled)

7. A GIP analogue according to claim 1 wherein Ψ is a residue of Lys, Arg, Orn or Cys in which the side chain is conjugated to a substituent having the formula —Z¹ or —Z²—Z¹.

8. A GIP analogue according to claim 7 wherein —Z¹ is a fatty chain having at a terminus a connection —X— to Ψ or to Z²;
wherein —X— is a bond, —CO—, —SO—, or —SO₂—;
and, optionally, Z¹ has a polar group at the end of the chain distal from connection —X—; said polar group comprising a carboxylic acid or a carboxylic acid bioisostere, a phosphonic acid, or a sulfonic acid group.

9. (canceled)

10. A GIP analogue according to claim 7 wherein Z¹ is:
A-B-C₁₆₋₂₀alkylene-(CO)— wherein A is H or —COOH and B is a bond, for example:
17-carboxy-heptadecanoyl HOOC—(CH₂)₁₆—(CO)—;
19-carboxy-nonadecanoyl HOOC—(CH₂)₁₈—(CO)—;
Octadecanoyl H₃C—(CH₂)₁₆—(CO)—; or
Eicosanoyl H₃C—(CH₂)₁₈—(CO)—.

11. A GIP analogue according to claim 7 wherein Z² is a spacer bound at one terminus by Y, which is a nitrogen, oxygen or sulfur atom, and at the other terminus by X, which is a bond or an acyl (—CO—), sulfinyl (—SO—), sulfonyl (—SO₂—) or absent.

12. A GIP analogue according to claim 7 wherein —Z¹—Z² is:

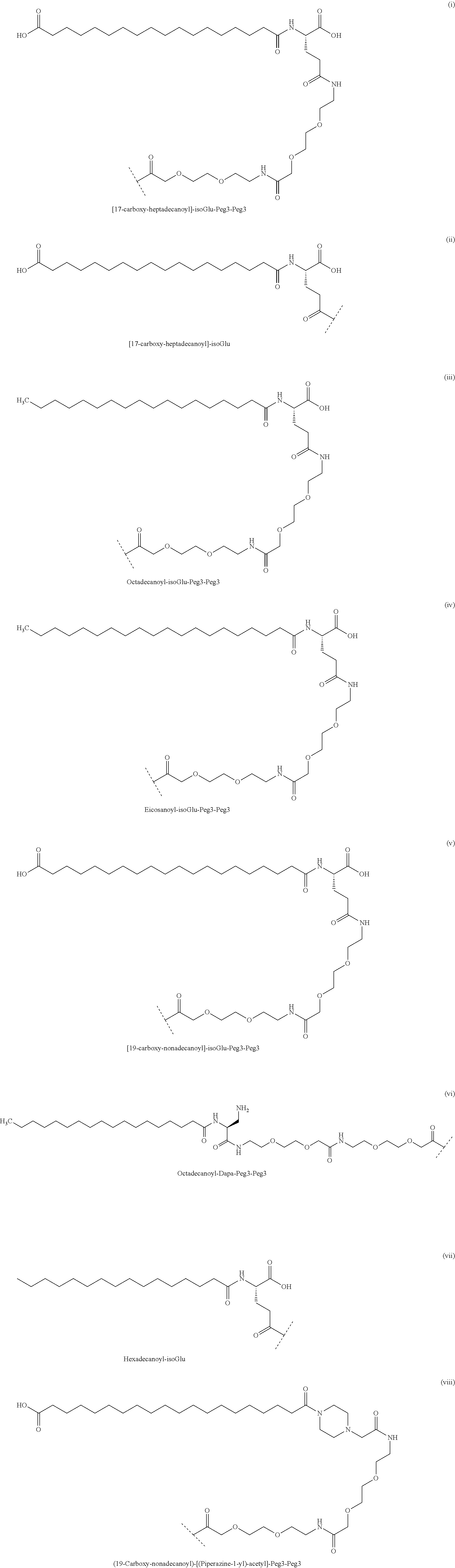

13. A GIP analogue according to claim 1 having the sequence
(SEQ ID NO: 51)
Y-Aib-EGTFISDYSIELDKΨHQQDFVNWLLAQGPSSGAPPPS;
(SEQ ID NO: 52)
Y-Aib-EGTFISDYSIELDΨIHQQDFVNWLLAQGPSSGAPPPS;
(SEQ ID NO: 53)
Y-Aib-EGTFISDYSIELEKΨHQQDFVNWLLAQGPSSGAPPPS;
(SEQ ID NO: 54)
Y-Aib-EGTFISDYSIELDKIHQQDFVNWLLAQGPSSGAPPPSΨ;
(SEQ ID NO: 55)
Y-Aib-EGTFISDYSIELDKIHQQDFVNWLLAQΨ;
(SEQ ID NO: 56)
Y-Aib-EGTFISDYSIELDKIHQQDFVNWLLAQKGΨ;
(SEQ ID NO:57)
Y-Aib-EGTFISDYSIELDKΨHQQDFVNYLLAQGPSSGAPPPS;
(SEQ ID NO: 58)
Y-Aib-EGTFISDYSIELDKΨHQQDFVNWLLAQGPSSGAPPPS;
(SEQ ID NO: 59)
Y-Aib-EGTFISDYSIELDKΨAAQDFVNWLLAQGPSSGAPPPS;
(SEQ ID NO: 60)
Y-Aib-EGTFISDYSIELEKΨAAKEFVNWLLAQGPSSGAPPPS;
(SEQ ID NO: 61)
Y-Aib-EGTFISDYSIELEKΨAQRAFVEWLLAQGPSSGAPPPS;
(SEQ ID NO: 62)
Y-Aib-EGTFISDYSIELEKIAQRAFVEWLLAQGPSSGAPPPSΨ;
(SEQ ID NO: 63)
Y-Aib-EGTFISDYSIELEKIAQRAFVEWLLAQΨ;
(SEQ ID NO: 64)
Y-Aib-EGTFISDYSIELDKΨAAQDFVNWLLAGPSSGAPPPS;
(SEQ ID NO: 65)
Y-Aib-EGTFISDYSIELDKIAAQDFVNWLLAGPSSGAPPPSΨ;
(SEQ ID NO: 130)
Y-Aib-EGTFISDYSIELDKKΨAQRAFVEWLLAQGPSSGAPPPS;
(SEQ ID NO: 131)
Y-Aib-EGTFISDYSIELDKKΨAQRAFIEWLLAQGPSSGAPPPS;
(SEQ ID NO: 68)
Y-Aib-EGTFISDYSIELDKIAQRAFIEWLLAGPSSGAPPPSKΨ;
(SEQ ID NO: 69)
Y-Aib-EGTFISDYSIELDKIAQKEFIEWLLAGPSSGAPPPSKΨ;
(SEQ ID NO: 70)
Y-Aib-EGTFISDYSIELDKIAAQDFIEWLLAGPSSGAPPPSKΨ;
(SEQ ID NO: 71)
Y-Aib-EGTFISDYSIELDKIAAQDFVEWLLAGPSSGAPPPSKΨ;
(SEQ ID NO: 72)
Y-Aib-EGTFISDYSIELDKIAQRAFIEWLLAQGPSSGAPPPSKΨ;
(SEQ ID NO: 132)
Y-Aib-EGTFISDYSIELDKKΨAAQAFVNWLLAGPSSGAPPPS;
(SEQ ID NO: 133)
Y-Aib-EGTFISDYSIELDKKΨAAQDFVNWLLAAGPSSGAPPPS;
(SEQ ID NO: 134)
Y-Aib-EGTFISDYSIELDKKΨAAQDFINWLLAGPSSGAPPPS;
(SEQ ID NO: 135)
Y-Aib-EGTFISDYSIELDKKΨAAQDFIEWLLAGPSSGAPPPS;
(SEQ ID NO: 135)
Y-Aib-EGTFISDYSIELDKKΨAAQDFIEWLLAGPSSGAPPPS;
(SEQ ID NO: 78)
Y-Aib-EGTFISDYSIELDKΨIAQRAFIEWLLAQGPSSGAPPPS;
(SEQ ID NO: 136)
Y-Aib-EGTFISDYSKΨELDKIAQRAFIEWLLAQGPSSGAPPPS;
(SEQ ID NO: 137)
Y-DAla-EGTFISDYSIELDKKΨAQRAFIEWLLAQGPSSGAPPPS;
(SEQ ID NO: 81)
Y-DAla-EGTFISDYSIELDKIAAQDFIEWLLAGPSSGAPPPSKW;
(SEQ ID NO: 138)
Y-Aib-EGTFISDYSIELDKKΨAAQDFIEWLLAQGPSSGAPPPS;
(SEQ ID NO: 139)
Y-Aib-EGTFISDYSIELDKKΨAAQDFINWLLAQGPSSGAPPPS;
or
(SEQ ID NO: 140)
Y-Aib-EGTFISDYSIELDKKΨAAQAFIEWLLAQGPSSGAPPPS.

14. A GIP analogue according to claim 1 having the sequence
(SEQ ID NO: 1)
Y-Aib-EGTFISDYSIELDK-K(Hexadecanoyl-isoGlu)-
HQQDFVNWLLAQGPSSGAPPPS;
(SEQ ID NO: 2)
Y-Aib-EGTFISDYSIELD-K(Hexadecanoyl-isoGlu)-I
HQQDFVNWLLAQGPSSGAPPPS;
(SEQ ID NO: 3)
Y-Aib-EGTFISDYSIELEK-K(Hexadecanoyl-isoGlu)-
HQQDFVNWLLAQGPSSGAPPPS;
(SEQ ID NO: 4)
Y-Aib-EGTFISDYSIELDKIHQQDFVNWLLAQGPSSGAPPPS-
K([19-carboxy-nonadecanoyl]-isoGlu-
Peg3-Peg3);
(SEQ ID NO: 5)
Y-Aib-EGTFISDYSIELDKIHQQDFVNWLLAQGPSSGAPPPS-
K(Hexadecanoyl-isoGlu);
(SEQ ID NO: 6)
Y-Aib-EGTFISDYSIELDKIHQQDFVNWLLAQ-
K(Hexadecanoyl-isoGlu);
(SEQ ID NO: 7)
Y-Aib-EGTFISDYSIELDKIHQQDFVNWLLAQKG-K
(Hexadecanoyl-isoGlu);
(SEQ ID NO: 8)
Y-Aib-EGTFISDYSIELDK-K([19-carboxy-
nonadecanoyl]-isoGlu-Peg3-Peg3)-
HQQDFVNYLLAQGPSSGAPPPS;
(SEQ ID NO: 9)
Y-Aib-EGTFISDYSIELDK-K([19-carboxy-
nonadecanoyl]-isoGlu-Peg3-Peg3)-
HQQDFVNWLLAQGPSSGAPPPS;
(SEQ ID NO: 10)
Y-Aib-EGTFISDYSIELDK-K([19-carboxy-
nonadecanoyl]-isoGlu-Peg3-Peg3)-
AAQDFVNWLLAQGPSSGAPPPS;
(SEQ ID NO: 11)
Y-Aib-EGTFISDYSIELEK-K([19-carboxy-
nonadecanoyl]-isoGlu-Peg3-Peg3)-
AAKEFVNWLLAQGPSSGAPPPS;
(SEQ ID NO: 12)
Y-Aib-EGTFISDYSIELEK-K([19-carboxy-
nonadecanoyl]-isoGlu-Peg3-Peg3)-
AQRAFVEWLLAQGPSSGAPPPS;
(SEQ ID NO: 13)
Y-Aib-EGTFISDYSIELEKIAQRAFVEWLLAQ
GPSSGAPPPS-K([19-carboxy-
nonadecanoyl]-isoGlu-Peg3-Peg3);
(SEQ ID NO: 141)
[[H-]]Y-Aib-EGTFISDYSIELEKIAQRAFV
EWLLAQ-K([19-carboxy-nonadecanoyl]-
isoGlu-Peg3-Peg3);
(SEQ ID NO: 15)
[[H-]]Y-Aib-EGTFISDYSIELDK-K([19-
carboxy-nonadecanoyl]-isoGlu-Peg3-Peg3)-
AAQDFVNWLLAGPSSGAPPPS;
(SEQ ID NO: 142)
[[H-]]Y-Aib-EGTFISDYSIELDKIAAQDFVNWLL
AGPSSGAPPPS-K([19-carboxy-nonadecanoyl]-
isoGlu-Peg3-Peg3);
(SEQ ID NO: 17)
Y-Aib-EGTFISDYSIELDK-K([19-Carboxy-
nonadecanoyl]-isoGlu-Peg3-Peg3)-
AQRAFVEWLLAQGPSSGAPPPS;
(SEQ ID NO: 18)
Y-Aib-EGTFISDYSIELDK-K([19-Carboxy-
nonadecanoyl]-isoGlu-Peg3-Peg3)-
AQRAFIEWLLAQGPSSGAPPPS;
(SEQ ID NO: 19)
Y-Aib-EGTFISDYSIELDK-K((19-Carboxy-
nonadecanoyl)-[(Piperazine-1-yl)-
acetyl]-Peg3-Peg3)-AQRAFIEWLLAQ
GPSSGAPPPS;
(SEQ ID NO: 20)
Y-Aib-EGTFISDYSIELDK-K((19-Carboxy-
nonadecanoyl)-[(Piperazine-1-yl)-
acetyl]-Peg3-Peg3)-AQRAFVEWLLA
QGPSSGAPPPS;
(SEQ ID NO: 21)
Y-Aib-EGTFISDYSIELDK-K((19-Carboxy-
nonadecanoyl)-[(Piperazine-1-yl)-
acetyl]-Peg3-Peg3)-AQKEFVEWLLA
AGPSSGAPPPS
(SEQ ID NO: 22)
Y-Aib-EGTFISDYSIELDK-K([19-Carboxy-
nonadecanoyl]-isoGlu-Peg3-Peg3)-
AQKEFVEWLLAAGPSSGAPPPS;
(SEQ ID NO: 23)
Y-Aib-EGTFISDYSIELDKIAQRAFIEWLLAGP
SSGAPPPS-K([19-Carboxy-nonadecanoyl]-
isoGlu-Peg3-Peg3);
(SEQ ID NO: 24)
Y-Aib-EGTFISDYSIELDKIAQKEFIEWLLAGPS
SGAPPPS-K([19-Carboxy-nonadecanoyl]-
isoGlu-Peg3-Peg3);
(SEQ ID NO: 25)
Y-Aib-EGTFISDYSIELDKIAAQDFIEWLLAGPS
SGAPPPS-K([19-Carboxy-nonadecanoyl]-
isoGlu-Peg3-Peg3);
(SEQ ID NO: 26)
Y-Aib-EGTFISDYSIELDKIAAQDFIEWLLAGP
SSGAPPPS-K((19-Carboxy-nonadecanoyl)-
[(Piperazine-1-yl)-acetyl]-Peg3-
Peg3);
(SEQ ID NO: 27)
Y-Aib-EGTFISDYSIELDKIAAQDFVEWLLAGPS
SGAPPPS-K([19-Carboxy-nonadecanoyl]-
isoGlu-Peg3-Peg3);
(SEQ ID NO: 28)
Y-Aib-EGTFISDYSIELDKIAQRAFIEWLLAQGP
SSGAPPPS-K([19-Carboxy-nonadecanoyl]-
isoGlu-Peg3-Peg3);
(SEQ ID NO: 29)
Y-Aib-EGTFISDYSIELDK-K([19-Carboxy-
nonadecanoyl]-isoGlu-Peg3-Peg3)-
AAQAFVNWLLAGPSSGAPPPS;
(SEQ ID NO: 30)
Y-Aib-EGTFISDYSIELDK-K([19-Carboxy-
nonadecanoyl]-isoGlu-Peg3-Peg3)-
AAQDFVNWLLAAGPSSGAPPPS;
(SEQ ID NO: 31)
Y-Aib-EGTFISDYSIELDK-K([19-Carboxy-
nonadecanoyl]-isoGlu-Peg3-Peg3)-
AAQDFINWLLAGPSSGAPPPS;
(SEQ ID NO: 32)
Y-Aib-EGTFISDYSIELDK-K([19-Carboxy-
nonadecanoyl]-isoGlu-Peg3-Peg3)-
AAQDFIEWLLAGPSSGAPPPS;
(SEQ ID NO: 33)
Y-Aib-EGTFISDYSIELDK-K((19-Carboxy-
nonadecanoyl)-[(Piperazine-1-yl)-
acetyl]-Peg3-Peg3)-AAQDFIEWLLAGPSS
GAPPPS;
(SEQ ID NO: 34)
Y-Aib-EGTFISDYSIELD-K([19-Carboxy-
nonadecanoyl]-isoGlu-Peg3-Peg3)-
IAQRAFIEWLLAQGPSSGAPPPS;
(SEQ ID NO: 35)
Y-Aib-EGTFISDYS-K([19-Carboxy-
nonadecanoyl]-isoGlu-Peg3-Peg3)-
ELDKIAQRAFIEWLLAQGPSSGAPPPS;
(SEQ ID NO: 36)
Y-DAla-EGTFISDYSIELDK-K([19-Carboxy-
nonadecanoyl]-isoGlu-Peg3-Peg3)-
AQRAFIEWLLAQGPSSGAPPPS;
(SEQ ID NO: 37)
Y-DAla-EGTFISDYSIELDKIAAQDFIEWLLAG
PSSGAPPPS-K((19-Carboxy-nonadecanoyl)-
[(Piperazine-1-yl)-acetyl]-Peg3-Peg3);
(SEQ ID NO: 38)
Y-Aib-EGTFISDYSIELDK-K([19-Carboxy-
nonadecanoyl]-isoGlu-Peg3-Peg3)-
AAQDFIEWLLAQGPSSGAPPPS;
(SEQ ID NO: 39)
Y-Aib-EGTFISDYSIELDK-K([19-Carboxy-
nonadecanoyl]-isoGlu-Peg3-Peg3)-
AAQDFINWLLAQGPSSGAPPPS;
(SEQ ID NO: 40)
Y-Aib-EGTFISDYSIELDK-K([19-Carboxy-
nonadecanoyl]-isoGlu-Peg3-Peg3)-
AAQAFIEWLLAQGPSSGAPPPS;
or
(SEQ ID NO: 41)
Y-Aib-EGTFISDYSIELDK-K((19-Carboxy-
nonadecanoyl)-[(Piperazine-1-yl)-
acetyl]-Peg3-Peg3)-AAQAFIEWLLAQGP
SSGAPPPS.

15. A GIP analogue according to claim 1 which is:
(SEQ ID NO: 89)
H-Y-Aib-EGTFISDYSIELDK-K(Hexadecanoyl-isoGlu)-
HQQDFVNWLLAQGPSSGAPPPS-NH₂;
(SEQ ID NO: 90)
H-Y-Aib-EGTFISDYSIELD-K(Hexadecanoyl-isoGlu)-I
HQQDFVNWLLAQGPSSGAPPPS-NH₂;
(SEQ ID NO: 91)
H-Y-Aib-EGTFISDYSIELEK-K(Hexadecanoyl-isoGlu)-
HQQDFVNWLLAQGPSSGAPPPS-NH₂;
(SEQ ID NO: 92)
H-Y-Aib-EGTFISDYSIELDKIHQQDFVNWLLAQGPSSGAPPPS-
K([19-carboxy-nonadecanoyl]-isoGlu-
Peg3-Peg3)-NH₂;
(SEQ ID NO: 93)
YH--Aib-EGTFISDYSIELDKIHQQDFVNWLLAQGPSSGAPPPS-
K(Hexadecanoyl-isoGlu)-NH₂;
(SEQ ID NO: 94)
H-Y-Aib-EGTFISDYSIELDKIHQQDFVNWLLAQ-
K(Hexadecanoyl-isoGlu)-NH₂;
(SEQ ID NO: 95)
H-Y-Aib-EGTFISDYSIELDKIHQQDFVNWLLAQKG-K
(Hexadecanoyl-isoGlu)-NH₂;
(SEQ ID NO: 96)
H-Y-Aib-EGTFISDYSIELDK-K([19-carboxy-
nonadecanoyl]-isoGlu-Peg3-Peg3)-
HQQDFVNYLLAQGPSSGAPPPS-NH₂;
(SEQ ID NO: 97)
H-Y-Aib-EGTFISDYSIELDK-K([19-carboxy-
nonadecanoyl]-isoGlu-Peg3-Peg3)-
HQQDFVNWLLAQGPSSGAPPPS;
(SEQ ID NO: 98)
H-Y-Aib-EGTFISDYSIELDK-K([19-carboxy-
nonadecanoyl]-isoGlu-Peg3-Peg3)-
AAQDFVNWLLAQGPSSGAPPPS-NH₂;
(SEQ ID NO: 99)
H-Y-Aib-EGTFISDYSIELEK-K([19-carboxy-
nonadecanoyl]-isoGlu-Peg3-Peg3)-
AAKEFVNWLLAQGPSSGAPPPS-NH₂;
(SEQ ID NO: 100)
H-Y-Aib-EGTFISDYSIELEK-K([19-carboxy-
nonadecanoyl]-isoGlu-Peg3-Peg3)-
AQRAFVEWLLAQGPSSGAPPPS-NH₂;
(SEQ ID NO: 101)
H-Y-Aib-EGTFISDYSIELEKIAQRAFVEWLLAQ
GPSSGAPPPS-K([19-carboxy-
nonadecanoyl]-isoGlu-Peg3-Peg3)-NH₂;
(SEQ ID NO: 102)
H-Y-Aib-EGTFISDYSIELEKIAQRAFV
EWLLAQ-K([19-carboxy-nonadecanoyl]-
isoGlu-Peg3-Peg3)-NH₂;
(SEQ ID NO: 103)
H-Y-Aib-EGTFISDYSIELDK-K([19-
carboxy-nonadecanoyl]-isoGlu-Peg3-Peg3)-
AAQDFVNWLLAGPSSGAPPPS-NH₂;
(SEQ ID NO: 104)
H-Y-Aib-EGTFISDYSIELDKIAAQDFVNWLL
AGPSSGAPPPS-K([19-carboxy-nonadecanoyl]-
isoGlu-Peg3-Peg3)-NH₂;
(SEQ ID NO: 105)
H-Y-Aib-EGTFISDYSIELDK-K([19-Carboxy-
nonadecanoyl]-isoGlu-Peg3-Peg3)-
AQRAFVEWLLAQGPSSGAPPPS-NH₂;
(SEQ ID NO: 106)
H-Y-Aib-EGTFISDYSIELDK-K([19-Carboxy-
nonadecanoyl]-isoGlu-Peg3-Peg3)-
AQRAFIEWLLAQGPSSGAPPPS-NH₂;
(SEQ ID NO: 107)
H-Y-Aib-EGTFISDYSIELDK-K((19-Carboxy-
nonadecanoyl)-[(Piperazine-1-yl)-
acetyl]-Peg3-Peg3)-AQRAFIEWLLAQ
GPSSGAPPPS-NH₂;
(SEQ ID NO: 108)
H-Y-Aib-EGTFISDYSIELDK-K((19-Carboxy-
nonadecanoyl)-[(Piperazine-1-yl)-
acetyl]-Peg3-Peg3)-AQRAFVEWLLA
QGPSSGAPPPS-NH₂;
(SEQ ID NO: 109)
H-Y-Aib-EGTFISDYSIELDK-K((19-Carboxy-
nonadecanoyl)-[(Piperazine-1-yl)-
acetyl]-Peg3-Peg3)-AQKEFVEWLLA
AGPSSGAPPPS-NH₂
(SEQ ID NO: 110)
H-Y-Aib-EGTFISDYSIELDK-K([19-Carboxy-
nonadecanoyl]-isoGlu-Peg3-Peg3)-
AQKEFVEWLLAAGPSSGAPPPS-NH₂;
(SEQ ID NO: 111)
H-Y-Aib-EGTFISDYSIELDKIAQRAFIEWLLAGP
SSGAPPPS-K([19-Carboxy-nonadecanoyl]-
isoGlu-Peg3-Peg3)-NH₂;
(SEQ ID NO: 112)
H-Y-Aib-EGTFISDYSIELDKIAQKEFIEWLLAGPS
SGAPPPS-K([19-Carboxy-nonadecanoyl]-
isoGlu-Peg3-Peg3)-NH₂;
(SEQ ID NO: 113)
H-Y-Aib-EGTFISDYSIELDKIAAQDFIEWLLAGPS
SGAPPPS-K([19-Carboxy-nonadecanoyl]-
isoGlu-Peg3-Peg3)-NH₂;
(SEQ ID NO: 114)
H-Y-Aib-EGTFISDYSIELDKIAAQDFIEWLLAGP
SSGAPPPS-K((19-Carboxy-nonadecanoyl)-
[(Piperazine-1-yl)-acetyl]-Peg3-
Peg3)-NH₂;
(SEQ ID NO: 115)
H-Y-Aib-EGTFISDYSIELDKIAAQDFVEWLLAGPS
SGAPPPS-K([19-Carboxy-nonadecanoyl]-
isoGlu-Peg3-Peg3)-NH₂;
(SEQ ID NO: 116)
H-Y-Aib-EGTFISDYSIELDKIAQRAFIEWLLAQGP
SSGAPPPS-K([19-Carboxy-nonadecanoyl]-
isoGlu-Peg3-Peg3)-NH₂;
(SEQ ID NO: 117)
H-Y-Aib-EGTFISDYSIELDK-K([19-Carboxy-
nonadecanoyl]-isoGlu-Peg3-Peg3)-
AAQAFVNWLLAGPSSGAPPPS-NH₂;
(SEQ ID NO: 118)
H-Y-Aib-EGTFISDYSIELDK-K([19-Carboxy-
nonadecanoyl]-isoGlu-Peg3-Peg3)-
AAQDFVNWLLAAGPSSGAPPPS-NH₂;
(SEQ ID NO: 119)
H-Y-Aib-EGTFISDYSIELDK-K([19-Carboxy-
nonadecanoyl]-isoGlu-Peg3-Peg3)-
AAQDFINWLLAGPSSGAPPPS-NH₂;
(SEQ ID NO: 120)
H-Y-Aib-EGTFISDYSIELDK-K([19-Carboxy-
nonadecanoyl]-isoGlu-Peg3-Peg3)-
AAQDFIEWLLAGPSSGAPPPS-NH₂;
(SEQ ID NO: 121)
H-Y-Aib-EGTFISDYSIELDK-K((19-Carboxy-
nonadecanoyl)-[(Piperazine-1-yl)-
acetyl]-Peg3-Peg3)-AAQDFIEWLLAGPSS
GAPPPS-NH₂;
(SEQ ID NO: 122)
H-Y-Aib-EGTFISDYSIELD-K([19-Carboxy-
nonadecanoyl]-isoGlu-Peg3-Peg3)-
IAQRAFIEWLLAQGPSSGAPPPS-NH₂;
(SEQ ID NO: 123)
H-Y-Aib-EGTFISDYS-K([19-Carboxy-
nonadecanoyl]-isoGlu-Peg3-Peg3)-
ELDKIAQRAFIEWLLAQGPSSGAPPPS-NH₂;
(SEQ ID NO: 124)
H-Y-DAla-EGTFISDYSIELDK-K([19-Carboxy-
nonadecanoyl]-isoGlu-Peg3-Peg3)-
AQRAFIEWLLAQGPSSGAPPPS-NH₂;
(SEQ ID NO: 125)
H-Y-DAla-EGTFISDYSIELDKIAAQDFIEWLLAG
PSSGAPPPS-K((19-Carboxy-nonadecanoyl)-
[(Piperazine-1-yl)-acetyl]-Peg3-Peg3)-NH₂;
(SEQ ID NO: 126)
H-Y-Aib-EGTFISDYSIELDK-K([19-Carboxy-
nonadecanoyl]-isoGlu-Peg3-Peg3)-
AAQDFIEWLLAQGPSSGAPPPS-NH₂;
(SEQ ID NO: 127)
H-Y-Aib-EGTFISDYSIELDK-K([19-Carboxy-
nonadecanoyl]-isoGlu-Peg3-Peg3)-
AAQDFINWLLAQGPSSGAPPPS-NH₂;
(SEQ ID NO: 128)
H-Y-Aib-EGTFISDYSIELDK-K([19-Carboxy-
nonadecanoyl]-isoGlu-Peg3-Peg3)-
AAQAFIEWLLAQGPSSGAPPPS-NH₂;
or
(SEQ ID NO: 129)
H-Y-Aib-EGTFISDYSIELDK-K((19-Carboxy-
nonadecanoyl)-[(Piperazine-1-yl)-
acetyl]-Peg3-Peg3)-AAQAFIEWLLAQGP
SSGAPPPS-NH₂.

16. A pharmaceutical composition comprising a GIP analogue according to claim 1, or a pharmaceutically acceptable salt or solvate thereof, in admixture with a carrier.

17. A pharmaceutical composition according to claim 16 formulated as a liquid suitable for administration by injection or infusion,

18. (canceled)

19. A method of medical treatment comprising administering a GIP analogue according to claim 1, or a pharmaceutically acceptable salt or solvate thereof, to a subject.

20. A method of treatment and/or prevention of a metabolic disorder in a subject, the method comprising administering a GIP analogue according to claim 1, or a pharmaceutically acceptable salt or solvate thereof, to a subject.

21. A method according to claim 20 where the GIP analogue is administered as part of a combination therapy with an anti-diabetic agent.

22. A method GIP according to claim 20 where the GIP analogue is administered as part of a combination therapy with a GLP-1 agonist.

23. A method according to claim 20 wherein the metabolic disorder is diabetes or a diabetes related disorder, or obesity or an obesity related disorder.

24. A method according to claim 23 wherein
(a) the diabetes related disorder is insulin resistance, glucose intolerance, increased fasting glucose, hypoglycemia (for example induced by insulin treatment), pre-diabetes, type 1 diabetes, type 2 diabetes, gestational diabetes hypertension, dyslipidemia, or a combination thereof; or
(b) the diabetes related disorder is atherosclerosis, arteriosclerosis, coronary heart disease, peripheral artery disease, stroke; or is a condition associated with atherogenic dyslipidemia, a blood fat disorder, elevated blood pressure, hypertension, a prothrombotic state, or a proinflammatory state;
(c) the diabetes related disorder is osteoporosis including increased risk of bone fractures; or
(d) the obesity related disorder is obesity linked inflammation, obesity linked gallbladder disease or obesity induced sleep apnea, or may be associated with a condition selected from atherogenic dyslipidemia, blood fat disorders, elevated blood pressure, hypertension, a prothrombotic state, and a proinflammatory state, or a combination thereof.

25-26. (canceled)

27. A method according to claim 24 wherein
(a) the blood fat disorder is high triglycerides, low HDL cholesterol, high LDL cholesterol, plaque buildup in artery walls, or a combination thereof;
(b) the prothrombotic state comprises high fibrinogen levels in the blood or high plasminogen activator inhibitor-1 levels in the blood; and/or
(c) the proinflammatory state comprises an elevated C-reactive protein level in the blood.

28-30. (canceled)


SEQUENCE LISTING (142 sequences)

SEQ ID NO: 1 - protein, 39 aa, synthetic construct
  source 1..39: note = Synthetic GIP analogue
  SITE 2: note = MOD_RES - Aib - Aminoisobutyric acid
  SITE 17: note = Lys(Hexadecanoyl-isoGlu) - Hexadecanoyl-Isoglutamic acid is attached to the side chain of Lysine
YXEGTFISDYSIELDKKHQQDFVNWLLAQGPSSGAPPPS

SEQ ID NO: 2 - protein, 39 aa, synthetic construct
  source 1..39: note = Synthetic GIP analogue
  SITE 2: note = MOD_RES - Aib - Aminoisobutyric acid
  SITE 16: note = Lys(Hexadecanoyl-isoGlu) - Hexadecanoyl-Isoglutamic acid is attached to the side chain of Lysine
YXEGTFISDYSIELDKIHQQDFVNWLLAQGPSSGAPPPS

SEQ ID NO: 3 - protein, 39 aa, synthetic construct
  source 1..39: note = Synthetic GIP analogue
  SITE 2: note = MOD_RES - Aib - Aminoisobutyric acid
  SITE 17: note = Lys(Hexadecanoyl-isoGlu) - Hexadecanoyl-Isoglutamic acid is attached to the side chain of Lysine
YXEGTFISDYSIELEKKHQQDFVNWLLAQGPSSGAPPPS

SEQ ID NO: 4 - protein, 40 aa, synthetic construct
  source 1..40: note = Synthetic GIP analogue
  SITE 2: note = MOD_RES - Aib - Aminoisobutyric acid
  SITE 40: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKIHQQDFVNWLLAQGPSSGAPPPSK

SEQ ID NO: 5 - protein, 40 aa, synthetic construct; identical to SEQ ID NO: 4
  source 1..40: note = Synthetic GIP analogue
  SITE 2: note = MOD_RES - Aib - Aminoisobutyric acid
  SITE 40: note = Lys(Hexadecanoyl-isoGlu) - Hexadecanoyl-Isoglutamic acid is attached to the side chain of Lysine

SEQ ID NO: 6 - protein, 30 aa, synthetic construct
  source 1..30: note = Synthetic GIP analogue
  SITE 2: note = MOD_RES - Aib - Aminoisobutyric acid
  SITE 30: note = Lys(Hexadecanoyl-isoGlu) - Hexadecanoyl-Isoglutamic acid is attached to the side chain of Lysine
YXEGTFISDYSIELDKIHQQDFVNWLLAQK

SEQ ID NO: 7 - protein, 32 aa, synthetic construct
  source 1..32: note = Synthetic GIP analogue
  SITE 2: note = MOD_RES - Aib - Aminoisobutyric acid
  SITE 32: note = Lys(Hexadecanoyl-isoGlu) - Hexadecanoyl-Isoglutamic acid is attached to the side chain of Lysine
YXEGTFISDYSIELDKIHQQDFVNWLLAQKGK

SEQ ID NO: 8 - protein, 39 aa, synthetic construct
  source 1..39: note = Synthetic GIP analogue
  SITE 2: note = MOD_RES - Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKKHQQDFVNYLLAQGPSSGAPPPS

SEQ ID NO: 9 - protein, 39 aa, synthetic construct; identical to SEQ ID NO: 1
  source 1..39: note = Synthetic GIP analogue
  SITE 2: note = MOD_RES - Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine

SEQ ID NO: 10 - protein, 39 aa, synthetic construct
  source 1..39: note = Synthetic GIP analogue
  SITE 2: note = MOD_RES - Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKKAAQDFVNWLLAQGPSSGAPPPS

SEQ ID NO: 11 - protein, 39 aa, synthetic construct
  source 1..39: note = Synthetic GIP analogue
  SITE 2: note = MOD_RES - Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELEKKAAKEFVNWLLAQGPSSGAPPPS

SEQ ID NO: 12 - protein, 39 aa, synthetic construct
  source 1..39: note = Synthetic GIP analogue
  SITE 2: note = MOD_RES - Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELEKKAQRAFVEWLLAQGPSSGAPPPS

SEQ ID NO: 13 - protein, 40 aa, synthetic construct
  source 1..40: note = Synthetic GIP analogue
  SITE 2: note = MOD_RES - Aib - Aminoisobutyric acid
  SITE 40: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELEKIAQRAFVEWLLAQGPSSGAPPPSK

SEQ ID NO: 14 - protein, 30 aa, synthetic construct
  source 1..30: note = Synthetic GIP analogue
  SITE 2: note = MOD_RES - Aib - Aminoisobutyric acid
  SITE 30: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELEKIAQRAFVEWLLAQK

SEQ ID NO: 15 - protein, 38 aa, synthetic construct
  source 1..38: note = Synthetic GIP analogue
  SITE 2: note = MOD_RES - Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKKAAQDFVNWLLAGPSSGAPPPS

SEQ ID NO: 16 - protein, 39 aa, synthetic construct
  source 1..39: note = Synthetic GIP analogue
  SITE 2: note = MOD_RES - Aib - Aminoisobutyric acid
  SITE 39: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKIAAQDFVNWLLAGPSSGAPPPSK

SEQ ID NO: 17 - protein, 39 aa, synthetic construct
  source 1..39: note = Synthetic GIP analogue
  SITE 2: note = MOD_RES - Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKKAQRAFVEWLLAQGPSSGAPPPS

SEQ ID NO: 18 - protein, 39 aa, synthetic construct
  source 1..39: note = Synthetic GIP analogue
  SITE 2: note = MOD_RES - Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKKAQRAFIEWLLAQGPSSGAPPPS

SEQ ID NO: 19 - protein, 39 aa, synthetic construct; identical to SEQ ID NO: 18
  source 1..39: note = Synthetic GIP analogue
  SITE 2: note = MOD_RES - Aib - Aminoisobutyric acid
  SITE 17: note = Lys((19-Carboxy-nonadecanoyl)-((Piperazine-1-yl)-ac etyl)-Peg3-Peg3) - (19-Carboxy-nonadecanoyl)-((Piperazine-1-yl)-acetyl)-3x polyethylene glycol-3x polyethylene glycol is attached to the side chain of Lysine

SEQ ID NO: 20 - protein, 39 aa, synthetic construct; identical to SEQ ID NO: 17
  source 1..39: note = Synthetic GIP analogue
  SITE 2: note = MOD_RES - Aib - Aminoisobutyric acid
  SITE 17: note = Lys((19-Carboxy-nonadecanoyl)-((Piperazine-1-yl)-ac etyl)-Peg3-Peg3) - (19-Carboxy-nonadecanoyl)-((Piperazine-1-yl)-acetyl)-3x polyethylene glycol-3x polyethylene glycol is attached to the side chain of Lysine

SEQ ID NO: 21 - protein, 39 aa, synthetic construct
  source 1..39: note = Synthetic GIP analogue
  SITE 2: note = MOD_RES - Aib - Aminoisobutyric acid
  SITE 17: note = Lys((19-Carboxy-nonadecanoyl)-((Piperazine-1-yl)-ac etyl)-Peg3-Peg3) - (19-Carboxy-nonadecanoyl)-((Piperazine-1-yl)-acetyl)-3x polyethylene glycol-3x polyethylene glycol is attached to the side chain of Lysine
YXEGTFISDYSIELDKKAQKEFVEWLLAAGPSSGAPPPS

SEQ ID NO: 22 - protein, 39 aa, synthetic construct; identical to SEQ ID NO: 21
  source 1..39: note = Synthetic GIP analogue
  SITE 2: note = MOD_RES - Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine

SEQ ID NO: 23 - protein, 39 aa, synthetic construct
  source 1..39: note = Synthetic GIP analogue
  SITE 2: note = MOD_RES - Aib - Aminoisobutyric acid
  SITE 39: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKIAQRAFIEWLLAGPSSGAPPPSK

SEQ ID NO: 24 - protein, 39 aa, synthetic construct
  source 1..39: note = Synthetic GIP analogue
  SITE 2: note = MOD_RES - Aib - Aminoisobutyric acid
  SITE 39: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKIAQKEFIEWLLAGPSSGAPPPSK

SEQ ID NO: 25 - protein, 39 aa, synthetic construct
  source 1..39: note = Synthetic GIP analogue
  SITE 2: note = MOD_RES - Aib - Aminoisobutyric acid
  SITE 39: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKIAAQDFIEWLLAGPSSGAPPPSK

SEQ ID NO: 26 - protein, 39 aa, synthetic construct; identical to SEQ ID NO: 25
  source 1..39: note = Synthetic GIP analogue
  SITE 2: note = MOD_RES - Aib - Aminoisobutyric acid
  SITE 39: note = Lys((19-Carboxy-nonadecanoyl)-((Piperazine-1-yl)-ac etyl)-Peg3-Peg3) - (19-Carboxy-nonadecanoyl)-((Piperazine-1-yl)-acetyl)-3x polyethylene glycol-3x polyethylene glycol is attached to the side chain of Lysine

SEQ ID NO: 27 - protein, 39 aa, synthetic construct
  source 1..39: note = Synthetic GIP analogue
  SITE 2: note = MOD_RES - Aib - Aminoisobutyric acid
  SITE 39: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKIAAQDFVEWLLAGPSSGAPPPSK

SEQ ID NO: 28 - protein, 40 aa, synthetic construct
  source 1..40: note = Synthetic GIP analogue
  SITE 2: note = MOD_RES - Aib - Aminoisobutyric acid
  SITE 40: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKIAQRAFIEWLLAQGPSSGAPPPSK

SEQ ID NO: 29 - protein, 38 aa, synthetic construct
  source 1..38: note = Synthetic GIP analogue
  SITE 2: note = MOD_RES - Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKKAAQAFVNWLLAGPSSGAPPPS

SEQ ID NO: 30 - protein, 39 aa, synthetic construct
  source 1..39: note = Synthetic GIP analogue
  SITE 2: note = MOD_RES - Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKKAAQDFVNWLLAAGPSSGAPPPS

SEQ ID NO: 31 - protein, 38 aa, synthetic construct
  source 1..38: note = Synthetic GIP analogue
  SITE 2: note = MOD_RES - Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKKAAQDFINWLLAGPSSGAPPPS

SEQ ID NO: 32 - protein, 38 aa, synthetic construct
  source 1..38: note = Synthetic GIP analogue
  SITE 2: note = MOD_RES - Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKKAAQDFIEWLLAGPSSGAPPPS

SEQ ID NO: 33 - protein, 38 aa, synthetic construct; identical to SEQ ID NO: 32
  source 1..38: note = Synthetic GIP analogue
  SITE 2: note = MOD_RES - Aib - Aminoisobutyric acid
  SITE 17: note = Lys((19-Carboxy-nonadecanoyl)-((Piperazine-1-yl)-ac etyl)-Peg3-Peg3) - (19-Carboxy-nonadecanoyl)-((Piperazine-1-yl)-acetyl)-3x polyethylene glycol-3x polyethylene glycol is attached to the side chain of Lysine

SEQ ID NO: 34 - protein, 39 aa, synthetic construct
  source 1..39: note = Synthetic GIP analogue
  SITE 2: note = MOD_RES - Aib - Aminoisobutyric acid
  SITE 16: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKIAQRAFIEWLLAQGPSSGAPPPS

SEQ ID NO: 35 - protein, 39 aa, synthetic construct
  source 1..39: note = Synthetic GIP analogue
  SITE 2: note = MOD_RES - Aib - Aminoisobutyric acid
  SITE 12: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSKELDKIAQRAFIEWLLAQGPSSGAPPPS

SEQ ID NO: 36 - protein, 39 aa, synthetic construct
  source 1..39: note = Synthetic GIP analogue
  SITE 2: note = D-Alanine
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YAEGTFISDYSIELDKKAQRAFIEWLLAQGPSSGAPPPS

SEQ ID NO: 37 - protein, 39 aa, synthetic construct
  source 1..39: note = Synthetic GIP analogue
  SITE 2: note = D-Alanine
  SITE 39: note = Lys((19-Carboxy-nonadecanoyl)-((Piperazine-1-yl)-ac etyl)-Peg3-Peg3) - (19-Carboxy-nonadecanoyl)-((Piperazine-1-yl)-acetyl)-3x polyethylene glycol-3x polyethylene glycol is attached to the side chain of Lysine
YAEGTFISDYSIELDKIAAQDFIEWLLAGPSSGAPPPSK

SEQ ID NO: 38 - protein, 39 aa, synthetic construct
  source 1..39: note = Synthetic GIP analogue
  SITE 2: note = MOD_RES - Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKKAAQDFIEWLLAQGPSSGAPPPS

SEQ ID NO: 39 - protein, 39 aa, synthetic construct
  source 1..39: note = Synthetic GIP analogue
  SITE 2: note = MOD_RES - Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKKAAQDFINWLLAQGPSSGAPPPS

SEQ ID NO: 40 - protein, 39 aa, synthetic construct
  source 1..39: note = Synthetic GIP analogue
  SITE 2: note = MOD_RES - Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
YXEGTFISDYSIELDKKAAQAFIEWLLAQGPSSGAPPPS

SEQ ID NO: 41 - protein, 39 aa, synthetic construct; identical to SEQ ID NO: 40
  source 1..39: note = Synthetic GIP analogue
  SITE 2: note = MOD_RES - Aib - Aminoisobutyric acid
  SITE 17: note = Lys((19-Carboxy-nonadecanoyl)-((Piperazine-1-yl)-ac etyl)-Peg3-Peg3) - (19-Carboxy-nonadecanoyl)-((Piperazine-1-yl)-acetyl)-3x polyethylene glycol-3x polyethylene glycol is attached to the side chain of Lysine

SEQ ID NO: 42 - protein, 43 aa, synthetic construct
  source 1..43: note = Synthetic GIP analogue of general Formula I
  REGION 1..43: note = VARIANT - The GIP analogue contains one and only one residue Psi; wherein Psi is a residue independently selected from Lys, Arg, Orn or Cys and wherein the side chain of said residue is conjugated to a lipophilic substituent
  VARIANT 1: note = SITE - pGlu - pyroglutamic acid or absent
  VARIANT 2: note = SITE - If position 1 is absent, then this residue can be modified by Acetyl, C1-4 alkyl, formyl, benzoyl or trifluoroacetyl
  VARIANT 3: note = Xaa is Aib - Aminoisobutyric acid, Ala, D-Alanine, Gly, Ser, N-Me-Ser - N-methyl-Serine, Ac3c - 1-Aminocyclopropane-1-carboxylic acid, Ac4c - 1-aminocyclobutane carboxylic acid or Ac5c - 1-aminocyclopentane carboxylic acid
  VARIANT 11: note = Xaa is Tyr, Leu or Ser
  VARIANT 12: note = Xaa is Ser or Leu
  VARIANT 13: note = Xaa is Lys, Psi or Ile; wherein Psi is a residue independently selected from Lys, Arg, Orn or Cys and wherein the side chain of said residue is conjugated to a lipophilic substituent
  VARIANT 16: note = Xaa is Asp or Glu
  VARIANT 17: note = Xaa is Ser, Glu, Lys or Psi; wherein Psi is a residue independently selected from Lys, Arg, Orn or Cys and wherein the side chain of said residue is conjugated to a lipophilic substituent
  VARIANT 18: note = Xaa is Ile, Lys, Gln, Arg or Psi; wherein Psi is a residue independently selected from Lys, Arg, Orn or Cys and wherein the side chain of said residue is conjugated to a lipophilic substituent
  VARIANT 19: note = Xaa is His, Arg or Ala
  VARIANT 20: note = Xaa is Gln, Lys, Ala or Glu
  VARIANT 21: note = Xaa is Gln, Lys, Ala, His or Arg
  VARIANT 22: note = Xaa is Ala, Leu, Asp or Glu
  VARIANT 24: note = Xaa is Val or Ile
  VARIANT 25: note = Xaa is Asn or Glu
  VARIANT 26: note = Xaa is Tyr or Trp
  VARIANT 28: note = Xaa is Leu, Glu, Ser, Lys or Val
  VARIANT 29: note = Xaa is Ala, Ser or Arg
  VARIANT 30: note = Xaa is Aib - Aminoisobutyric acid, Gly, Ala, Gln, Thr, Ser or Lys or is absent
  VARIANT 31..42: note = This region may encompass one of the following sequences: starting at pos. 31, is: KG (pos. 31-32) or GPSSGAPPPS or GPSSGAPPS or PSSGAPPPS or PSSGAPPS or GKKNDWKHNITQ or may be absent
  VARIANT 31..43: note = Xaa is Psi (as defined in 'VARIANT' for positions 13, 17 and 18) or absent; This applies to positions 31, 33, 39, 40, 41 or 43, dependent upon which chain of amino acids starts at pos. 31 as noted in the 'VARIANT' for positions 31-42 (listed above).
  VARIANT 43: note = Xaa is Psi; wherein Psi is a residue independently selected from Lys, Arg, Orn or Cys that may be amidated and wherein the side chain of said residue is conjugated to a lipophilic substituent or may be absent
XYXEGTFISDXXXELXXXXXXXFXXXLXXXXXXXXXXXXXXXX

SEQ ID NO: 43 - protein, 10 aa, synthetic construct
  source 1..10: note = Synthetic sequence Y1 in SEQ ID NOs: 42, 50, 87, and 89
GPSSGAPPPS

SEQ ID NO: 44 - protein, 9 aa, synthetic construct
  source 1..9: note = Synthetic sequence Y1 in SEQ ID NOs: 42, 48, 50, and 87 - 89
GPSSGAPPS

SEQ ID NO: 45 - protein, 9 aa, synthetic construct
  source 1..9: note = Synthetic sequence Y1 in SEQ ID NOs: 42, 48, 50, and 87 - 89
PSSGAPPPS

SEQ ID NO: 46 - protein, 8 aa, synthetic construct
  source 1..8: note = Synthetic sequence Y1 in SEQ ID NOs: 42, 48, 50, and 87 - 89
PSSGAPPS

SEQ ID NO: 47 - protein, 12 aa, synthetic construct
  source 1..12: note = Synthetic sequence Y1 in SEQ ID NOs: 42, 48, 50, and 87 - 89
GKKNDWKHNITQ

SEQ ID NO: 48 - protein, 43 aa, synthetic construct
  source 1..43: note = Synthetic GIP analogue of general Formula II
  REGION 1..43: note = VARIANT - The GIP analogue contains one and only one residue Psi; wherein Psi is a Lys residue wherein the side chain of said Lys residue is conjugated to a lipophilic substituent
  VARIANT 1: note = SITE - pGlu - pyroglutamic acid or absent
  VARIANT 2: note = SITE - If position 1 is absent, then this residue can be modified by Acetyl
  VARIANT 3: note = Xaa is Aib - Aminoisobutyric acid, Ala, D-Alanine, or Gly
  VARIANT 13: note = Xaa is Lys, Psi or Ile; wherein Psi is a Lys residue wherein the side chain of said Lys residue is conjugated to a lipophilic substituent
  VARIANT 16: note = Xaa is Asp or Glu
  VARIANT 17: note = Xaa is Ser, Glu, Lys or Psi; wherein Psi is a Lys residue wherein the side chain of said Lys residue is conjugated to a lipophilic substituent
  VARIANT 18: note = Xaa is Ile, Lys, Gln, Arg or Psi; wherein Psi is a Lys residue wherein the side chain of said Lys residue is conjugated to a lipophilic substituent
  VARIANT 19: note = Xaa is His, Arg or Ala
  VARIANT 20: note = Xaa is Gln or Ala
  VARIANT 21: note = Xaa is Gln, Lys, Ala, His or Arg
  VARIANT 22: note = Xaa is Ala, Asp or Glu
  VARIANT 24: note = Xaa is Ile or Val
  VARIANT 25: note = Xaa is Asn or Glu
  VARIANT 26: note = Xaa is Tyr or Trp
  VARIANT 28: note = Xaa is Leu, Glu, Ser, Lys or Val
  VARIANT 29: note = Xaa is Ala, Ser or Arg
  VARIANT 30: note = Xaa is Aib - Aminoisobutyric acid, Gly, Ala, Gln, Thr, Ser or Lys or is absent
  VARIANT 31..42: note = This region may encompass the one of the following sequences starting at position 31, is: KG (pos. 31-32) or GPSSGAPPPS or GPSSGAPPS or PSSGAPPPS or PSSGAPPS or GKKNDWKHNITQ or may be absent
  VARIANT 31..43: note = Xaa is Psi (as defined in 'VARIANT' for positions 13, 17 and 18) or absent; This applies to positions 31, 33, 39, 40, or 43, dependent upon which chain of amino acids starts at position 31 as noted in the 'VARIANT' for positions 31-42 (listed above).
  VARIANT 43: note = Xaa is Psi; wherein Psi is a Lys residue that may be amidated wherein the side chain of said Lys residue is conjugated to a lipophilic substituent or may be absent
XYXEGTFISDYSXELXXXXXXXFXXXLXXXXXXXXXXXXXXXX

SEQ ID NO: 49 - protein, 9 aa, synthetic construct
  source 1..9: note = Synthetic sequence Y1 in SEQ ID NOs: 48 and 88
GPSSGAPPP

SEQ ID NO: 50 - protein, 43 aa, synthetic construct
  source 1..43: note = Synthetic GIP analogue of general Formula III
  REGION 1..43: note = VARIANT - The GIP analogue contains one and only one residue Psi; wherein Psi is a residue independently selected from Lys, Arg, Orn or Cys and wherein the side chain of said residue is conjugated to a lipophilic substituent
  VARIANT 1: note = SITE - pGlu - pyroglutamic acid or absent
  VARIANT 2: note = SITE - If position 1 is absent, then this residue can be modified by Acetyl
  SITE 3: note = MOD_RES - Aib - Aminoisobutyric acid
  VARIANT 16: note = Xaa is Asp or Glu
  VARIANT 17: note = Xaa is Lys or Psi; wherein Psi is a residue independently selected from Lys, Arg, Orn or Cys and wherein the side chain of said residue is conjugated to a lipophilic substituent
  VARIANT 18: note = Xaa is Ile or Psi; wherein Psi is a residue independently selected from Lys, Arg, Orn or Cys and wherein the side chain of said residue is conjugated to a lipophilic substituent
  VARIANT 19: note = Xaa is His or Ala
  VARIANT 20: note = Xaa is Gln or Ala
  VARIANT 21: note = Xaa is Gln, Lys or Arg
  VARIANT 22: note = Xaa is Ala, Asp or Glu
  VARIANT 25: note = Xaa is Asn or Glu
  VARIANT 26: note = Xaa is Tyr or Trp
  VARIANT 30: note = Xaa is Gln or is absent
  VARIANT 31..42: note = This region may encompass the one of the following sequences starting at pos. 31, is: KG (pos. 31-32) or GPSSGAPPPS or GPSSGAPPS or PSSGAPPPS or PSSGAPPS or GKKNDWKHNITQ or may be absent
  VARIANT 31..43: note = Xaa is Psi (as defined in 'VARIANT' for positions 17 and 18) or absent; This applies to positions 31, 33, 39, 40, 41 or 43, dependent upon which chain of amino acids starts at pos. 31 as noted in the 'VARIANT' for positions 31-42 (listed above).
  VARIANT 43: note = Xaa is Psi; wherein Psi is a residue independently selected from Lys, Arg, Orn or Cys that may be amidated and wherein the side chain of said residue is conjugated to a lipophilic substituent or may be absent
XYXEGTFISDYSIELXXXXXXXFVXXLLAXXXXXXXXXXXXXX

SEQ ID NO: 51 - protein, 39 aa, synthetic construct
  source 1..39: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent
YXEGTFISDYSIELDKXHQQDFVNWLLAQGPSSGAPPPS

SEQ ID NO: 52 - protein, 39 aa, synthetic construct
  source 1..39: note = Synthetic GIP analogue
  SITE 2: note = MOD_RES - Aib - Aminoisobutyric acid
  VARIANT 16: note = Xaa is Psi, wherein Psi is a residue independently selected from Lys, Arg, Orn or Cys and wherein the side chain of said residue is conjugated to a lipophilic substituent
YXEGTFISDYSIELDXIHQQDFVNWLLAQGPSSGAPPPS

SEQ ID NO: 53 - protein, 39 aa, synthetic construct
  source 1..39: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent
YXEGTFISDYSIELEKXHQQDFVNWLLAQGPSSGAPPPS

SEQ ID NO: 54 - protein, 40 aa, synthetic construct
  source 1..40: note = Synthetic GIP analogue
  SITE 2: note = MOD_RES - Aib - Aminoisobutyric acid
  VARIANT 40: note = Xaa is Psi, wherein Psi is a residue independently selected from Lys, Arg, Orn or Cys and wherein the side chain of said residue is conjugated to a lipophilic substituent
YXEGTFISDYSIELDKIHQQDFVNWLLAQGPSSGAPPPSX

SEQ ID NO: 55 - protein, 30 aa, synthetic construct
  source 1..30: note = Synthetic GIP analogue
  SITE 2: note = MOD_RES - Aib - Aminoisobutyric acid
  VARIANT 30: note = Xaa is Psi, wherein Psi is a residue independently selected from Lys, Arg, Orn or Cys and wherein the side chain of said residue is conjugated to a lipophilic substituent
YXEGTFISDYSIELDKIHQQDFVNWLLAQX

SEQ ID NO: 56 - protein, 32 aa, synthetic construct
  source 1..32: note = Synthetic GIP analogue
  SITE 2: note = MOD_RES - Aib - Aminoisobutyric acid
  VARIANT 32: note = Xaa is Psi, wherein Psi is a residue independently selected from Lys, Arg, Orn or Cys and wherein the side chain of said residue is conjugated to a lipophilic substituent
YXEGTFISDYSIELDKIHQQDFVNWLLAQKGX

SEQ ID NO: 57 - protein, 39 aa, synthetic construct
  source 1..39: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent
YXEGTFISDYSIELDKXHQQDFVNYLLAQGPSSGAPPPS

SEQ ID NO: 58 - protein, 39 aa, synthetic construct; identical to SEQ ID NO: 51
  source 1..39: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent

SEQ ID NO: 59 - protein, 39 aa, synthetic construct
  source 1..39: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent
YXEGTFISDYSIELDKXAAQDFVNWLLAQGPSSGAPPPS

SEQ ID NO: 60 - protein, 39 aa, synthetic construct
  source 1..39: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent
YXEGTFISDYSIELEKXAAKEFVNWLLAQGPSSGAPPPS

SEQ ID NO: 61 - protein, 39 aa, synthetic construct
  source 1..39: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent
YXEGTFISDYSIELEKXAQRAFVEWLLAQGPSSGAPPPS

SEQ ID NO: 62 - protein, 40 aa, synthetic construct
  source 1..40: note = Synthetic GIP analogue
  SITE 2: note = MOD_RES - Aib - Aminoisobutyric acid
  VARIANT 40: note = Xaa is Psi, wherein Psi is a residue independently selected from Lys, Arg, Orn or Cys and wherein the side chain of said residue is conjugated to a lipophilic substituent
YXEGTFISDYSIELEKIAQRAFVEWLLAQGPSSGAPPPSX

SEQ ID NO: 63 - protein, 30 aa, synthetic construct
  source 1..30: note = Synthetic GIP analogue
  SITE 2: note = MOD_RES - Aib - Aminoisobutyric acid
  VARIANT 30: note = Xaa is Psi, wherein Psi is a residue independently selected from Lys, Arg, Orn or Cys and wherein the side chain of said residue is conjugated to a lipophilic substituent
YXEGTFISDYSIELEKIAQRAFVEWLLAQX

SEQ ID NO: 64 - protein, 38 aa, synthetic construct
  source 1..38: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent
YXEGTFISDYSIELDKXAAQDFVNWLLAGPSSGAPPPS

SEQ ID NO: 65 - protein, 39 aa, synthetic construct
  source 1..39: note = Synthetic GIP analogue
  SITE 2: note = MOD_RES - Aib - Aminoisobutyric acid
  VARIANT 39: note = Xaa is Psi, wherein Psi is a residue independently selected from Lys, Arg, Orn or Cys and wherein the side chain of said residue is conjugated to a lipophilic substituent
YXEGTFISDYSIELDKIAAQDFVNWLLAGPSSGAPPPSX

SEQ ID NO: 66 - protein, 39 aa, synthetic construct
  source 1..39: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent
YXEGTFISDYSIELDKXAQRAFVEWLLAQGPSSGAPPPS

SEQ ID NO: 67 - protein, 39 aa, synthetic construct
  source 1..39: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent
YXEGTFISDYSIELDKXAQRAFIEWLLAQGPSSGAPPPS

SEQ ID NO: 68 - protein, 40 aa, synthetic construct
  source 1..40: note = Synthetic GIP analogue
  SITE 2: note = MOD_RES - Aib - Aminoisobutyric acid
  VARIANT 40: note = Xaa is Psi, wherein Psi is a residue independently selected from Lys, Arg, Orn or Cys and wherein the side chain of said residue is conjugated to a lipophilic substituent
YXEGTFISDYSIELDKIAQRAFIEWLLAGPSSGAPPPSKX

SEQ ID NO: 69 - protein, 40 aa, synthetic construct
  source 1..40: note = Synthetic GIP analogue
  SITE 2: note = MOD_RES - Aib - Aminoisobutyric acid
  VARIANT 40: note = Xaa is Psi, wherein Psi is a residue independently selected from Lys, Arg, Orn or Cys and wherein the side chain of said residue is conjugated to a lipophilic substituent
YXEGTFISDYSIELDKIAQKEFIEWLLAGPSSGAPPPSKX

SEQ ID NO: 70 - protein, 40 aa, synthetic construct
  source 1..40: note = Synthetic GIP analogue
  SITE 2: note = MOD_RES - Aib - Aminoisobutyric acid
  VARIANT 40: note = Xaa is Psi, wherein Psi is a residue independently selected from Lys, Arg, Orn or Cys and wherein the side chain of said residue is conjugated to a lipophilic substituent
YXEGTFISDYSIELDKIAAQDFIEWLLAGPSSGAPPPSKX

SEQ ID NO: 71 - protein, 40 aa, synthetic construct
  source 1..40: note = Synthetic GIP analogue
  SITE 2: note = MOD_RES - Aib - Aminoisobutyric acid
  VARIANT 40: note = Xaa is Psi, wherein Psi is a residue independently selected from Lys, Arg, Orn or Cys and wherein the side chain of said residue is conjugated to a lipophilic substituent
YXEGTFISDYSIELDKIAAQDFVEWLLAGPSSGAPPPSKX

SEQ ID NO: 72 - protein, 41 aa, synthetic construct
  source 1..41: note = Synthetic GIP analogue
  SITE 2: note = MOD_RES - Aib - Aminoisobutyric acid
  VARIANT 41: note = Xaa is Psi, wherein Psi is a residue independently selected from Lys, Arg, Orn or Cys and wherein the side chain of said residue is conjugated to a lipophilic substituent
YXEGTFISDYSIELDKIAQRAFIEWLLAQGPSSGAPPPSKX

SEQ ID NO: 73 - protein, 38 aa, synthetic construct
  source 1..38: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent
YXEGTFISDYSIELDKXAAQAFVNWLLAGPSSGAPPPS

SEQ ID NO: 74 - protein, 39 aa, synthetic construct
  source 1..39: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent
YXEGTFISDYSIELDKXAAQDFVNWLLAAGPSSGAPPPS

SEQ ID NO: 75 - protein, 38 aa, synthetic construct
  source 1..38: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent
YXEGTFISDYSIELDKXAAQDFINWLLAGPSSGAPPPS

SEQ ID NO: 76 - protein, 38 aa, synthetic construct
  source 1..38: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent
YXEGTFISDYSIELDKXAAQDFIEWLLAGPSSGAPPPS

SEQ ID NO: 77 - protein, 38 aa, synthetic construct; identical to SEQ ID NO: 76
  source 1..38: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent

SEQ ID NO: 78 - protein, 40 aa, synthetic construct
  source 1..40: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent
YXEGTFISDYSIELDKXIAQRAFIEWLLAQGPSSGAPPPS

SEQ ID NO: 79 - protein, 39 aa, synthetic construct
  source 1..39: note = Synthetic GIP analogue
  SITE 2: note = MOD_RES - Aib - Aminoisobutyric acid
  VARIANT 12: note = Xaa is Psi, wherein Psi is a residue independently selected from Lys, Arg, Orn or Cys and wherein the side chain of said residue is conjugated to a lipophilic substituent
YXEGTFISDYSXELDKIAQRAFIEWLLAQGPSSGAPPPS

SEQ ID NO: 80 - protein, 39 aa, synthetic construct
  source 1..39: note = Synthetic GIP analogue
  SITE 2: note = D-Alanine
  VARIANT 17: note = Xaa is Psi, wherein Psi is a residue independently selected from Lys, Arg, Orn or Cys and wherein the side chain of said residue is conjugated to a lipophilic substituent
YAEGTFISDYSIELDKXAQRAFIEWLLAQGPSSGAPPPS

SEQ ID NO: 81 - protein, 40 aa, synthetic construct
  source 1..40: note = Synthetic GIP analogue
  SITE 2: note = D-Alanine
  VARIANT 40: note = Xaa is Psi, wherein Psi is a residue independently selected from Lys, Arg, Orn or Cys and wherein the side chain of said residue is conjugated to a lipophilic substituent
YAEGTFISDYSIELDKIAAQDFIEWLLAGPSSGAPPPSKX

SEQ ID NO: 82 - protein, 39 aa, synthetic construct
  source 1..39: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent
YXEGTFISDYSIELDKXAAQDFIEWLLAQGPSSGAPPPS

SEQ ID NO: 83 - protein, 39 aa, synthetic construct
  source 1..39: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent
YXEGTFISDYSIELDKXAAQDFINWLLAQGPSSGAPPPS

SEQ ID NO: 84 - protein, 39 aa, synthetic construct
  source 1..39: note = Synthetic GIP analogue
  SITE 2: note = MOD_RES - Aib - Aminoisobutyric acid
  VARIANT 17: note = Xaa is Psi, wherein Psi is a residue independently selected from Lys, Arg, Orn or Cys and wherein the side chain of said residue is conjugated to a lipophilic substituent
YXEGTFISDYSIELDKXAAQAFIEWLLAQGPSSGAPPPS

SEQ ID NO: 85 - protein, 6 aa, synthetic construct
  source 1..6: note = Synthetic linker
GSGSGG

SEQ ID NO: 86 - protein, 43 aa, synthetic construct; identical to SEQ ID NO: 42
  source 1..43: note = Synthetic GIP analogue of general Formula I
  REGION 1..43: note = VARIANT - The GIP analogue contains one and only one residue Psi; wherein Psi is a residue independently selected from Lys, Arg, Orn or Cys and wherein the side chain of said residue is conjugated to a lipophilic substituent
  VARIANT 1: note = SITE - pGlu - pyroglutamic acid or absent
  VARIANT 2: note = SITE - If position 1 is absent, then this residue can be modified by Acetyl, C1-4 alkyl, formyl, benzoyl or trifluoroacetyl
  VARIANT 3: note = Xaa is Aib - Aminoisobutyric acid, Ala, D-Alanine, Gly, Ser, N-Me-Ser - N-methyl-Serine, Ac3c - 1-Aminocyclopropane-1-carboxylic acid, Ac4c - 1-aminocyclobutane carboxylic acid or Ac5c - 1-aminocyclopentane carboxylic acid
  VARIANT 11: note = Xaa is Tyr, Leu or Ser
  VARIANT 12: note = Xaa is Ser or Leu
  VARIANT 13: note = Xaa is Lys, Psi or Ile; wherein Psi is a residue independently selected from Lys, Arg, Orn or Cys and wherein the side chain of said residue is conjugated to a lipophilic substituent
  VARIANT 16: note = Xaa is Asp or Glu
  VARIANT 17: note = Xaa is Ser, Glu, Lys or Psi; wherein Psi is a residue independently selected from Lys, Arg, Orn or Cys and wherein the side chain of said residue is conjugated to a lipophilic substituent
  VARIANT 18: note = Xaa is Ile, Lys, Gln, Arg or Psi; wherein Psi is a residue independently selected from Lys, Arg, Orn or Cys and wherein the side chain of said residue is conjugated to a lipophilic substituent
  VARIANT 19: note = Xaa is His, Arg or Ala
  VARIANT 20: note = Xaa is Gln, Lys, Ala or Glu
  VARIANT 21: note = Xaa is Gln, Lys, Ala, His or Arg
  VARIANT 22: note = Xaa is Ala, Leu, Asp or Glu
  VARIANT 24: note = Xaa is Val or Ile
  VARIANT 25: note = Xaa is Asn or Glu
  VARIANT 26: note = Xaa is Tyr or Trp
  VARIANT 28: note = Xaa is Leu, Glu, Ser, Lys or Val
  VARIANT 29: note = Xaa is Ala, Ser or Arg
  VARIANT 30: note = Xaa is Aib - Aminoisobutyric acid, Gly, Ala, Gln, Thr, Ser or Lys or is absent
  VARIANT 31..42: note = This region may encompass one of the following sequences: starting at pos. 31, is: KG (pos. 31-32) or GPSSGAPPPS or GPSSGAPPS or PSSGAPPPS or PSSGAPPS or GKKNDWKHNITQ or may be absent
  VARIANT 31..43: note = Xaa is Psi (as defined in 'VARIANT' for positions 13, 17 and 18) or absent; This applies to positions 31, 33, 39, 40, 41 or 43, dependent upon which chain of amino acids starts at pos. 31 as noted in the 'VARIANT' for positions 31-42 (listed above).
  VARIANT 43: note = Xaa is Psi; wherein Psi is a residue independently selected from Lys, Arg, Orn or Cys that may be amidated and wherein the side chain of said residue is conjugated to a lipophilic substituent or may be absent

SEQ ID NO: 87 - protein, 43 aa, synthetic construct; identical to SEQ ID NO: 48
  source 1..43: note = Synthetic GIP analogue of general Formula II
  REGION 1..43: note = VARIANT - The GIP analogue contains one and only one residue Psi; wherein Psi is a Lys residue wherein the side chain of said Lys residue is conjugated to a lipophilic substituent
  VARIANT 1: note = SITE - pGlu - pyroglutamic acid or absent
  VARIANT 2: note = SITE - If position 1 is absent, then this residue can be modified by Acetyl
  VARIANT 3: note = Xaa is Aib - Aminoisobutyric acid, Ala, D-Alanine, or Gly
  VARIANT 13: note = Xaa is Lys, Psi or Ile; wherein Psi is a Lys residue wherein the side chain of said Lys residue is conjugated to a lipophilic substituent
  VARIANT 16: note = Xaa is Asp or Glu
  VARIANT 17: note = Xaa is Ser, Glu, Lys or Psi; wherein Psi is a Lys residue wherein the side chain of said Lys residue is conjugated to a lipophilic substituent
  VARIANT 18: note = Xaa is Ile, Lys, Gln, Arg or Psi; wherein Psi is a Lys residue wherein the side chain of said Lys residue is conjugated to a lipophilic substituent
  VARIANT 19: note = Xaa is His, Arg or Ala
  VARIANT 20: note = Xaa is Gln or Ala
  VARIANT 21: note = Xaa is Gln, Lys, Ala, His or Arg
  VARIANT 22: note = Xaa is Ala, Asp or Glu
  VARIANT 24: note = Xaa is Ile or Val
  VARIANT 25: note = Xaa is Asn or Glu
  VARIANT 26: note = Xaa is Tyr or Trp
  VARIANT 28: note = Xaa is Leu, Glu, Ser, Lys or Val
  VARIANT 29: note = Xaa is Ala, Ser or Arg
  VARIANT 30: note = Xaa is Aib - Aminoisobutyric acid, Gly, Ala, Gln, Thr, Ser or Lys or is absent
  VARIANT 31..42: note = This region may encompass the one of the following sequences starting at position 31, is: KG (pos. 31-32) or GPSSGAPPPS or GPSSGAPPS or PSSGAPPPS or PSSGAPPS or GKKNDWKHNITQ or may be absent
  VARIANT 31..43: note = Xaa is Psi (as defined in 'VARIANT' for positions 13, 17 and 18) or absent; This applies to positions 31, 33, 39, 40, or 43, dependent upon which chain of amino acids starts at position 31 as noted in the 'VARIANT' for positions 31-42 (listed above).
  VARIANT 43: note = Xaa is Psi; wherein Psi is a Lys residue that may be amidated wherein the side chain of said Lys residue is conjugated to a lipophilic substituent or may be absent

SEQ ID NO: 88 - protein, 43 aa, synthetic construct; identical to SEQ ID NO: 50
  source 1..43: note = Synthetic GIP analogue of general Formula III
  REGION 1..43: note = VARIANT - The GIP analogue contains one and only one residue Psi; wherein Psi is a residue independently selected from Lys, Arg, Orn or Cys and wherein the side chain of said residue is conjugated to a lipophilic substituent
  VARIANT 1: note = SITE - pGlu - pyroglutamic acid or absent
  VARIANT 2: note = SITE - If position 1 is absent, then this residue can be modified by Acetyl
  SITE 3: note = MOD_RES - Aib - Aminoisobutyric acid
  VARIANT 16: note = Xaa is Asp or Glu
  VARIANT 17: note = Xaa is Lys or Psi; wherein Psi is a residue independently selected from Lys, Arg, Orn or Cys and wherein the side chain of said residue is conjugated to a lipophilic substituent
  VARIANT 18: note = Xaa is Ile or Psi; wherein Psi is a residue independently selected from Lys, Arg, Orn or Cys and wherein the side chain of said residue is conjugated to a lipophilic substituent
  VARIANT 19: note = Xaa is His or Ala
  VARIANT 20: note = Xaa is Gln or Ala
  VARIANT 21: note = Xaa is Gln, Lys or Arg
  VARIANT 22: note = Xaa is Ala, Asp or Glu
  VARIANT 25: note = Xaa is Asn or Glu
  VARIANT 26: note = Xaa is Tyr or Trp
  VARIANT 30: note = Xaa is Gln or is absent
  VARIANT 31..42: note = This region may encompass the one of the following sequences starting at pos. 31, is: KG (pos. 31-32) or GPSSGAPPPS or GPSSGAPPS or PSSGAPPPS or PSSGAPPS or GKKNDWKHNITQ or may be absent
  VARIANT 31..43: note = Xaa is Psi (as defined in 'VARIANT' for positions 17 and 18) or absent; This applies to positions 31, 33, 39, 40, 41 or 43, dependent upon which chain of amino acids starts at pos. 31 as noted in the 'VARIANT' for positions 31-42 (listed above).
  VARIANT 43: note = Xaa is Psi; wherein Psi is a residue independently selected from Lys, Arg, Orn or Cys that may be amidated and wherein the side chain of said residue is conjugated to a lipophilic substituent

SEQ ID NO: 89 - protein, 39 aa, synthetic construct; identical to SEQ ID NO: 1
  source 1..39: note = Synthetic GIP analogue
  SITE 2: note = Aib - Aminoisobutyric acid
  SITE 17: note = Lys(Hexadecanoyl-isoGlu) - Hexadecanoyl-Isoglutamic acid is attached to the side chain of Lysine
  SITE 39: note = Amidated Residue

SEQ ID NO: 90 - protein, 39 aa, synthetic construct; identical to SEQ ID NO: 2
  source 1..39: note = Synthetic GIP analogue
  SITE 2: note = Aib - Aminoisobutyric acid
  SITE 16: note = Lys(Hexadecanoyl-isoGlu) - Hexadecanoyl-Isoglutamic acid is attached to the side chain of Lysine
  SITE 39: note = Amidated Residue

SEQ ID NO: 91 - protein, 39 aa, synthetic construct; identical to SEQ ID NO: 3
  source 1..39: note = Synthetic GIP analogue
  SITE 2: note = Aib - Aminoisobutyric acid
  SITE 17: note = Lys(Hexadecanoyl-isoGlu) - Hexadecanoyl-Isoglutamic acid is attached to the side chain of Lysine
  SITE 39: note = Amidated Residue

SEQ ID NO: 92 - protein, 40 aa, synthetic construct; identical to SEQ ID NO: 4
  source 1..40: note = Synthetic GIP analogue
  SITE 2: note = Aib - Aminoisobutyric acid
  SITE 40: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of amidated Lysine

SEQ ID NO: 93 - protein, 40 aa, synthetic construct; identical to SEQ ID NO: 4
  source 1..40: note = Synthetic GIP analogue
  SITE 2: note = Aib - Aminoisobutyric acid
  SITE 40: note = Lys(Hexadecanoyl-isoGlu) - Hexadecanoyl-Isoglutamic acid is attached to the side chain of amidated Lysine

SEQ ID NO: 94 - protein, 30 aa, synthetic construct; identical to SEQ ID NO: 6
  source 1..30: note = Synthetic GIP analogue
  SITE 2: note = Aib - Aminoisobutyric acid
  SITE 30: note = Lys(Hexadecanoyl-isoGlu) - Hexadecanoyl-Isoglutamic acid is attached to the side chain of amidated Lysine

SEQ ID NO: 95 - protein, 32 aa, synthetic construct; identical to SEQ ID NO: 7
  source 1..32: note = Synthetic GIP analogue
  SITE 2: note = Aib - Aminoisobutyric acid
  SITE 32: note = Lys(Hexadecanoyl-isoGlu) - Hexadecanoyl-Isoglutamic acid is attached to the side chain of amidated Lysine

SEQ ID NO: 96 - protein, 39 aa, synthetic construct; identical to SEQ ID NO: 8
  source 1..39: note = Synthetic GIP analogue
  SITE 2: note = Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 39: note = Amidated Residue

SEQ ID NO: 97 - protein, 39 aa, synthetic construct; identical to SEQ ID NO: 1
  source 1..39: note = Synthetic GIP analogue
  SITE 2: note = Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 39: note = Amidated Residue

SEQ ID NO: 98 - protein, 39 aa, synthetic construct; identical to SEQ ID NO: 10
  source 1..39: note = Synthetic GIP analogue
  SITE 2: note = Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 39: note = Amidated Residue

SEQ ID NO: 99 - protein, 39 aa, synthetic construct; identical to SEQ ID NO: 11
  source 1..39: note = Synthetic GIP analogue
  SITE 2: note = Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 39: note = Amidated Residue

SEQ ID NO: 100 - protein, 39 aa, synthetic construct; identical to SEQ ID NO: 12
  source 1..39: note = Synthetic GIP analogue
  SITE 2: note = Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 39: note = Amidated Residue

SEQ ID NO: 101 - protein, 40 aa, synthetic construct; identical to SEQ ID NO: 13
  source 1..40: note = Synthetic GIP analogue
  SITE 2: note = Aib - Aminoisobutyric acid
  SITE 40: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of amidated Lysine

SEQ ID NO: 102 - protein, 30 aa, synthetic construct; identical to SEQ ID NO: 14
  source 1..30: note = Synthetic GIP analogue
  SITE 2: note = Aib - Aminoisobutyric acid
  SITE 30: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of amidated Lysine

SEQ ID NO: 103 - protein, 38 aa, synthetic construct; identical to SEQ ID NO: 15
  source 1..38: note = Synthetic GIP analogue
  SITE 2: note = Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 38: note = Amidated Residue

SEQ ID NO: 104 - protein, 39 aa, synthetic construct; identical to SEQ ID NO: 16
  source 1..39: note = Synthetic GIP analogue
  SITE 2: note = Aib - Aminoisobutyric acid
  SITE 39: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of amidated Lysine

SEQ ID NO: 105 - protein, 39 aa, synthetic construct; identical to SEQ ID NO: 17
  source 1..39: note = Synthetic GIP analogue
  SITE 2: note = Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 39: note = Amidated Residue

SEQ ID NO: 106 - protein, 39 aa, synthetic construct; identical to SEQ ID NO: 18
  source 1..39: note = Synthetic GIP analogue
  SITE 2: note = Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 39: note = Amidated Residue

SEQ ID NO: 107 - protein, 39 aa, synthetic construct; identical to SEQ ID NO: 18
  source 1..39: note = Synthetic GIP analogue
  SITE 2: note = Aib - Aminoisobutyric acid
  SITE 17: note = Lys((19-Carboxy-nonadecanoyl)-((Piperazine-1-yl)-ac etyl)-Peg3-Peg3) - (19-Carboxy-nonadecanoyl)-((Piperazine-1-yl)-acetyl)-3x polyethylene glycol-3x polyethylene glycol is attached to the side chain of Lysine
  SITE 39: note = Amidated Residue

SEQ ID NO: 108 - protein, 39 aa, synthetic construct; identical to SEQ ID NO: 17
  source 1..39: note = Synthetic GIP analogue
  SITE 2: note = Aib - Aminoisobutyric acid
  SITE 17: note = Lys((19-Carboxy-nonadecanoyl)-((Piperazine-1-yl)-ac etyl)-Peg3-Peg3) - (19-Carboxy-nonadecanoyl)-((Piperazine-1-yl)-acetyl)-3x polyethylene glycol-3x polyethylene glycol is attached to the side chain of Lysine
  SITE 39: note = Amidated Residue

SEQ ID NO: 109 - protein, 39 aa, synthetic construct; identical to SEQ ID NO: 21
  source 1..39: note = Synthetic GIP analogue
  SITE 2: note = Aib - Aminoisobutyric acid
  SITE 17: note = Lys((19-Carboxy-nonadecanoyl)-((Piperazine-1-yl)-ac etyl)-Peg3-Peg3) - (19-Carboxy-nonadecanoyl)-((Piperazine-1-yl)-acetyl)-3x polyethylene glycol-3x polyethylene glycol is attached to the side chain of Lysine
  SITE 39: note = Amidated Residue

SEQ ID NO: 110 - protein, 39 aa, synthetic construct; identical to SEQ ID NO: 21
  source 1..39: note = Synthetic GIP analogue
  SITE 2: note = Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 39: note = Amidated Residue

SEQ ID NO: 111 - protein, 39 aa, synthetic construct; identical to SEQ ID NO: 23
  source 1..39: note = Synthetic GIP analogue
  SITE 2: note = Aib - Aminoisobutyric acid
  SITE 39: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of amidated Lysine

SEQ ID NO: 112 - protein, 39 aa, synthetic construct; identical to SEQ ID NO: 24
  source 1..39: note = Synthetic GIP analogue
  SITE 2: note = Aib - Aminoisobutyric acid
  SITE 39: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of amidated Lysine

SEQ ID NO: 113 - protein, 39 aa, synthetic construct; identical to SEQ ID NO: 25
  source 1..39: note = Synthetic GIP analogue
  SITE 2: note = Aib - Aminoisobutyric acid
  SITE 39: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of amidated Lysine

SEQ ID NO: 114 - protein, 39 aa, synthetic construct; identical to SEQ ID NO: 25
  source 1..39: note = Synthetic GIP analogue
  SITE 2: note = Aib - Aminoisobutyric acid
  SITE 39: note = Lys((19-Carboxy-nonadecanoyl)-((Piperazine-1-yl)-ac etyl)-Peg3-Peg3) - (19-Carboxy-nonadecanoyl)-((Piperazine-1-yl)-acetyl)-3x polyethylene glycol-3x polyethylene glycol is attached to the side chain of amidated Lysine

SEQ ID NO: 115 - protein, 39 aa, synthetic construct; identical to SEQ ID NO: 27
  source 1..39: note = Synthetic GIP analogue
  SITE 2: note = Aib - Aminoisobutyric acid
  SITE 39: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of amidated Lysine

SEQ ID NO: 116 - protein, 40 aa, synthetic construct; identical to SEQ ID NO: 28
  source 1..40: note = Synthetic GIP analogue
  SITE 2: note = Aib - Aminoisobutyric acid
  SITE 40: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of amidated Lysine

SEQ ID NO: 117 - protein, 38 aa, synthetic construct; identical to SEQ ID NO: 29
  source 1..38: note = Synthetic GIP analogue
  SITE 2: note = Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 38: note = Amidated Residue

SEQ ID NO: 118 - protein, 39 aa, synthetic construct; identical to SEQ ID NO: 30
  source 1..39: note = Synthetic GIP analogue
  SITE 2: note = Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 39: note = Amidated Residue

SEQ ID NO: 119 - protein, 38 aa, synthetic construct; identical to SEQ ID NO: 31
  source 1..38: note = Synthetic GIP analogue
  SITE 2: note = Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 38: note = Amidated Residue

SEQ ID NO: 120 - protein, 38 aa, synthetic construct; identical to SEQ ID NO: 32
  source 1..38: note = Synthetic GIP analogue
  SITE 2: note = Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 38: note = Amidated Residue

SEQ ID NO: 121 - protein, 38 aa, synthetic construct; identical to SEQ ID NO: 32
  source 1..38: note = Synthetic GIP analogue
  SITE 2: note = Aib - Aminoisobutyric acid
  SITE 17: note = Lys((19-Carboxy-nonadecanoyl)-((Piperazine-1-yl)-ac etyl)-Peg3-Peg3) - (19-Carboxy-nonadecanoyl)-((Piperazine-1-yl)-acetyl)-3x polyethylene glycol-3x polyethylene glycol is attached to the side chain of Lysine
  SITE 38: note = Amidated Residue

SEQ ID NO: 122 - protein, 39 aa, synthetic construct; identical to SEQ ID NO: 34
  source 1..39: note = Synthetic GIP analogue
  SITE 2: note = Aib - Aminoisobutyric acid
  SITE 16: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 39: note = Amidated Residue

SEQ ID NO: 123 - protein, 39 aa, synthetic construct; identical to SEQ ID NO: 35
  source 1..39: note = Synthetic GIP analogue
  SITE 2: note = Aib - Aminoisobutyric acid
  SITE 12: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 39: note = Amidated Residue

SEQ ID NO: 124 - protein, 39 aa, synthetic construct; identical to SEQ ID NO: 36
  source 1..39: note = Synthetic GIP analogue
  SITE 2: note = D-Alanine
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 39: note = Amidated Residue

SEQ ID NO: 125 - protein, 39 aa, synthetic construct; identical to SEQ ID NO: 37
  source 1..39: note = Synthetic GIP analogue
  SITE 2: note = D-Alanine
  SITE 39: note = Lys((19-Carboxy-nonadecanoyl)-((Piperazine-1-yl)-ac etyl)-Peg3-Peg3) - (19-Carboxy-nonadecanoyl)-((Piperazine-1-yl)-acetyl)-3x polyethylene glycol-3x polyethylene glycol is attached to the side chain of amidated Lysine

SEQ ID NO: 126 - protein, 39 aa, synthetic construct; identical to SEQ ID NO: 38
  source 1..39: note = Synthetic GIP analogue
  SITE 2: note = Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 39: note = Amidated Residue

SEQ ID NO: 127 - protein, 39 aa, synthetic construct; identical to SEQ ID NO: 39
  source 1..39: note = Synthetic GIP analogue
  SITE 2: note = Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 39: note = Amidated Residue

SEQ ID NO: 128 - protein, 39 aa, synthetic construct; identical to SEQ ID NO: 40
  source 1..39: note = Synthetic GIP analogue
  SITE 2: note = Aib - Aminoisobutyric acid
  SITE 17: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine
  SITE 39: note = Amidated Residue

SEQ ID NO: 129 - protein, 39 aa, synthetic construct; identical to SEQ ID NO: 40
  source 1..39: note = Synthetic GIP analogue
  SITE 2: note = Aib - Aminoisobutyric acid
  SITE 17: note = Lys((19-Carboxy-nonadecanoyl)-((Piperazine-1-yl)-ac etyl)-Peg3-Peg3) - (19-Carboxy-nonadecanoyl)-((Piperazine-1-yl)-acetyl)-3x polyethylene glycol-3x polyethylene glycol is attached to the side chain of Lysine
  SITE 39: note = Amidated Residue

SEQ ID NO: 130 - protein, 40 aa, synthetic construct
  SITE 2: note = Aib - Aminoisobutyric acid
  VARIANT 18: note = Xaa is Psi, wherein Psi is a residue independently selected from Lys, Arg, Orn or Cys and wherein the side chain of said residue is conjugated to a lipophilic substituent
YXEGTFISDYSIELDKKXAQRAFVEWLLAQGPSSGAPPPS

SEQ ID NO: 131 - protein, 40 aa, synthetic construct
  SITE 2: note = Aib - Aminoisobutyric acid
  VARIANT 18: note = Xaa is Psi, wherein Psi is a residue independently selected from Lys, Arg, Orn or Cys and wherein the side chain of said residue is conjugated to a lipophilic substituent
YXEGTFISDYSIELDKKXAQRAFIEWLLAQGPSSGAPPPS

SEQ ID NO: 132 - protein, 39 aa, synthetic construct
  SITE 2: note = Aib - Aminoisobutyric acid
  VARIANT 18: note = Xaa is Psi, wherein Psi is a residue independently selected from Lys, Arg, Orn or Cys and wherein the side chain of said residue is conjugated to a lipophilic substituent
YXEGTFISDYSIELDKKXAAQAFVNWLLAGPSSGAPPPS

SEQ ID NO: 133 - protein, 40 aa, synthetic construct
  SITE 2: note = Aib - Aminoisobutyric acid
  VARIANT 18: note = Xaa is Psi, wherein Psi is a residue independently selected from Lys, Arg, Orn or Cys and wherein the side chain of said residue is conjugated to a lipophilic substituent
YXEGTFISDYSIELDKKXAAQDFVNWLLAAGPSSGAPPPS

SEQ ID NO: 134 - protein, 39 aa, synthetic construct
  SITE 2: note = Aib - Aminoisobutyric acid
  VARIANT 18: note = Xaa is Psi, wherein Psi is a residue independently selected from Lys, Arg, Orn or Cys and wherein the side chain of said residue is conjugated to a lipophilic substituent
YXEGTFISDYSIELDKKXAAQDFINWLLAGPSSGAPPPS

SEQ ID NO: 135 - protein, 39 aa, synthetic construct
  SITE 2: note = Aib - Aminoisobutyric acid
  VARIANT 18: note = Xaa is Psi, wherein Psi is a residue independently selected from Lys, Arg, Orn or Cys and wherein the side chain of said residue is conjugated to a lipophilic substituent
YXEGTFISDYSIELDKKXAAQDFIEWLLAGPSSGAPPPS

SEQ ID NO: 136 - protein, 40 aa, synthetic construct
  SITE 2: note = Aib - Aminoisobutyric acid
  VARIANT 13: note = Xaa is Psi, wherein Psi is a residue independently selected from Lys, Arg, Orn or Cys and wherein the side chain of said residue is conjugated to a lipophilic substituent
YXEGTFISDYSKXELDKIAQRAFIEWLLAQGPSSGAPPPS

SEQ ID NO: 137 - protein, 40 aa, synthetic construct
  SITE 2: note = D-Alanine
  VARIANT 18: note = Xaa is Psi, wherein Psi is a residue independently selected from Lys, Arg, Orn or Cys and wherein the side chain of said residue is conjugated to a lipophilic substituent
YAEGTFISDYSIELDKKXAQRAFIEWLLAQGPSSGAPPPS

SEQ ID NO: 138 - protein, 40 aa, synthetic construct
  SITE 2: note = Aib - Aminoisobutyric acid
  VARIANT 18: note = Xaa is Psi, wherein Psi is a residue independently selected from Lys, Arg, Orn or Cys and wherein the side chain of said residue is conjugated to a lipophilic substituent
YXEGTFISDYSIELDKKXAAQDFIEWLLAQGPSSGAPPPS

SEQ ID NO: 139 - protein, 40 aa, synthetic construct
  SITE 2: note = Aib - Aminoisobutyric acid
  VARIANT 18: note = Xaa is Psi, wherein Psi is a residue independently selected from Lys, Arg, Orn or Cys and wherein the side chain of said residue is conjugated to a lipophilic substituent
YXEGTFISDYSIELDKKXAAQDFINWLLAQGPSSGAPPPS

SEQ ID NO: 140 - protein, 40 aa, synthetic construct
  SITE 2: note = Aib - Aminoisobutyric acid
  VARIANT 18: note = Xaa is Psi, wherein Psi is a residue independently selected from Lys, Arg, Orn or Cys and wherein the side chain of said residue is conjugated to a lipophilic substituent
YXEGTFISDYSIELDKKXAAQAFIEWLLAQGPSSGAPPPS

SEQ ID NO: 141 - protein, 30 aa, synthetic construct; identical to SEQ ID NO: 14
  SITE 2: note = Aib - Aminoisobutyric acid
  SITE 30: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine

SEQ ID NO: 142 - protein, 39 aa, synthetic construct; identical to SEQ ID NO: 16
  SITE 2: note = Aib - Aminoisobutyric acid
  SITE 39: note = Lys((19-carboxy-nonadecanoyl)-isoGlu-Peg3-Peg3) - (19-carboxy-nonadecanoyl)-Isoglutamic acid-3x polyethylene glycol-3x polyethylene glycol is attached to the side chain of Lysine